SEQUENCE LISTING (20 sequences)

SEQ ID NO: 1 - protein, 662 aa, Homo sapiens
MKLSPQPSCVNLGMMWTWALWMLPSLCKFSLAALPAKPENISCVYYYRKNLTCTWSPGKETSYTQYTVKRTYAFGEKHDNCTTNSSTSENRASCSFFLPRITIPDNYTIEVEAENGDGVIKSHMTYWRLENIAKTEPPKIFRVKPVLGIKRMIQIEWIKPELAPVSSDLKYTLRFRTVNSTSWMEVNFAKNRKDKNQTYNLTGLQPFTEYVIALRCAVKESKFWSDWSQEKMGMTEEEAPCGLELWRVLKPAEADGRRPVRLLWKKARGAPVLEKTLGYNIWYYPESNTNLTETMNTTNQQLELHLGGESFWVSMISYNSLGKSPVATLRIPAIQEKSFQCIEVMQACVAEDQLVVKWQSSALDVNTWMIEWFPDVDSEPTTLSWESVSQATNWTIQQDKLKPFWCYNISVYPMLHDKVGEPYSIQAYAKEGVPSEGPETKVENIGVKTVTITWKEIPKSERKGIICNYTIFYQAEGGKGFSKTVNSSILQYGLESLKRKTSYIVQVMASTSAGGTNGTSINFKTLSFSVFEIILITSLIGGGLLILIILTVAYGLKKPNKLTHLCWPTVPNPAESSIATWHGDDFKDKLNLKESDDSVNTEDRILKPCSTPSDKLVIDKLVVNFGNVLQEIFTDEARTGQENNLGGEKNGTRILSSCPTSI

SEQ ID NO: 2 - protein, 732 aa, Homo sapiens
MMWTWALWMLPSLCKFSLAALPAKPENISCVYYYRKNLTCTWSPGKETSYTQYTVKRTYAFGEKHDNCTTNSSTSENRASCSFFLPRITIPDNYTIEVEAENGDGVIKSHMTYWRLENIAKTEPPKIFRVKPVLGIKRMIQIEWIKPELAPVSSDLKYTLRFRTVNSTSWMEVNFAKNRKDKNQTYNLTGLQPFTEYVIALRCAVKESKFWSDWSQEKMGMTEEEAPCGLELWRVLKPAEADGRRPVRLLWKKARGAPVLEKTLGYNIWYYPESNTNLTETMNTTNQQLELHLGGESFWVSMISYNSLGKSPVATLRIPAIQEKSFQCIEVMQACVAEDQLVVKWQSSALDVNTWMIEWFPDVDSEPTTLSWESVSQATNWTIQQDKLKPFWCYNISVYPMLHDKVGEPYSIQAYAKEGVPSEGPETKVENIGVKTVTITWKEIPKSERKGIICNYTIFYQAEGGKGFSKTVNSSILQYGLESLKRKTSYIVQVMASTSAGGTNGTSINFKTLSFSVFEIILITSLIGGGLLILIILTVAYGLKKPNKLTHLCWPTVPNPAESSIATWHGDDFKDKLNLKESDDSVNTEDRILKPCSTPSDKLVIDKLVVNFGNVLQEIFTDEARTGQENNLGGEKNGYVTCPFRPDCPLGKSFEELPVSPEIPPRKSQYLRSRMPEGTRPEAKEQLLFSGQSLVPDHLCEEGAPNPYLKNSVTAREFLVSEKLPEHTKGEV

SEQ ID NO: 3 - protein, 716 aa, Mus sp.
MWTLALWAFSFLCKFSLAVLPTKPENISCVFYFDRNLTCTWRPEKETNDTSYIVTLTYSYGKSNYSDNATEASYSFPRSCAMPPDICSVEVQAQNGDGKVKSDITYWHLISIAKTEPPIILSVNPICNRMFQIQWKPREKTRGFPLVCMLRFRTVNSSRWTEVNFENCKQVCNLTGLQAFTEYVLALRFRFNDSRYWSKWSKEETRVTMEEVPHVLDLWRILEPADMNGDRKVRLLWKKARGAPVLEKTFGYHIQYFAENSTNLTEINNITTQQYELLLMSQAHSVSVTSFNSLGKSQEAILRIPDVHEKTFQYIKSMKAYIAEPLLVVNWQSSIPAVDTWIVEWLPEAAMSKFPALSWESVSQVTNWTIEQDKLKPFTCYNISVYPVLGHRVGEPYSIQAYAKEGTPLKGPETRVENIGLRTATITWKEIPKSARNGFINNYTVFYQAEGGKELSKTVNSHALQCDLESLTRRTSYTVWVMASTRAGGTNGVRINFKTLSISVFEIVLLTSLVGGGLLLLSIKTVTFGLRKPNRLTPLCCPDVPNPAESSLATWLGDGFKKSNMKETGNSGDTEDVVLKPCPVPADLIDKLVVNFENFLEVVLTEEAGKGQASILGGEANEYVTSPSRPDGPPGKSFKEPSVLTEVASEDSHSTCSRMADEAYSELARQPSSSCQSPGLSPPREDQAQNPYLKNSVTTREFLVHENIPEHSKGEV

SEQ ID NO: 4 - protein, 735 aa, Macaca fascicularis
MMWTWALWMFPLLCKFGLAALPAKPENISCVYYYRKNLTCTWSPGKETSYTQYTAKRTYAFGKKHDNCTTSSSTSENRASCSFFLPRITIPDNYTIEVEAENGDGVIKSDMTCWRLEDIAKTEPPEIFSVKPVLGIKRMIRIEWIKPELAPVSSDLKYALRFRTVNSTSWMEVNFAKNRKDTNQTYNLMGLQAFTEYVVALRCAVKESKFWSDWSQEKMGMTEEEAPCGLELWRVLKPTEVDGRRPVRLLWKKARGAPVLEKTLGYNIWYFPENNTNLTETVNTTNQQLELHLGGESYWVSMISYNSLGKSPVTTLRIPAIQEKSFRCIEVMQACLAEDQLVVKWQSSALDVNTWMIEWFPDMDSEHPTLSWESVSQATNWTIQQDKLKPFWCYNISVYPMLHDKVGEPYSIQAYAKEGIPSKGPETKVENIGVKTVTITWKEIPKSERKGIICNYTIFYQAEGGKGFSKTVNSSILQYGLESLKRKTSYTVRVMASTSAGGINGTSINFKTLSFSVFEIILITSLIGGGLLILIILTVAYGLKKPNKLTHLCWPSVPNPAESSIATWRGDDFKDKLNLKESDDSVNTEDRILKPCSTPSDKLVIDKSVVNFGNVLQEMFTDEARTGQENNLGGEKNEYVTHPFRADCPLGKSFEELPVSPEIPPRKSQYLRSRMPEGTCLEAEEQLLVSGQSLESLAPDHVREAAAPNPYLKNSVTTREFLVSQKLPEHTKGEV

SEQ ID NO: 5 - protein, 445 aa, synthetic construct
QVQLVQSGAEVKKPGASVKVSCKASGYTFTGYIMNWVRQAPGQGLEWMGLINPYNGGTDYNPQFQDRVTITADKSTSTAYMELSSLRSEDTAVYYCARDGYDDGPYTLETWGQGTLVTVSSASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSQEDPEVQFNWYVDGVEVHNAKTKPREEQFNSTFRVVSVLTVVHQDWLNGKEYKCKVSNKGLPAPIEKTISKTKGQPREPQVYTLPPSQEEMTKNQVSLTCLVKGFYPSDIAVEWESNGQPENNYKTTPPMLDSDGSFFLYSKLTVDKSRWQEGNVFSCSVMHEALHNHYTQKSLSLSP

SEQ ID NO: 6 - protein, 214 aa, synthetic construct
DIQMTQSPSSLSASVGDRVTITCQASEDIYSFVAWYQQKPGKAPKLLIYNAQTEAQGVPSRFSGSGSGTDFTLTISSLQPEDFATYYCQHHYDSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 121 aa, synthetic construct
QVQLVQSGAEVKKPGASVKVSCKASGYTFTGYIMNWVRQAPGQGLEWMGLINPYNGGTDYNPQFQDRVTITADKSTSTAYMELSSLRSEDTAVYYCARDGYDDGPYTLETWGQGTLVTVSS

SEQ ID NO: 8 - protein, 5 aa, synthetic construct
GYIMN

SEQ ID NO: 9 - protein, 17 aa, synthetic construct
LINPYNGGTDYNPQFQD

SEQ ID NO: 10 - protein, 12 aa, synthetic construct
DGYDDGPYTLET

SEQ ID NO: 11 - protein, 108 aa, synthetic construct
DIQMTQSPSSLSASVGDRVTITCQASEDIYSFVAWYQQKPGKAPKLLIYNAQTEAQGVPSRFSGSGSGTDFTLTISSLQPEDFATYYCQHHYDSPLTFGGGTKVEIKR

SEQ ID NO: 12 - protein, 11 aa, synthetic construct
QASEDIYSFVA

SEQ ID NO: 13 - protein, 7 aa, synthetic construct
NAQTEAQ

SEQ ID NO: 14 - protein, 9 aa, synthetic construct
QHHYDSPLT

SEQ ID NO: 15 - DNA, 834 bp, Homo sapiens
  source 1..834: mol_type = genomic DNA
agaatataacagcactcccaaagaactgggtactcaacactgagcagatctgttctttgagctaaaaaccatgtgctgtaccaagagtttgctcctggctgctttgatgtcagtgctgctactccacctctgcggcgaatcagaagcagcaagcaactttgactgctgtcttggatacacagaccgtattcttcatcctaaatttattgtgggcttcacacggcagctggccaatgaaggctgtgacatcaatgctatcatctttcacacaaagaaaaagttgtctgtgtgcgcaaatccaaaacagacttgggtgaaatatattgtgcgtctcctcagtaaaaaagtcaagaacatgtaaaaactgtggcttttctggaatggaattggacatagcccaagaacagaaagaaccttgctggggttggaggtttcacttgcacatcatggagggtttagtgcttatctaatttgtgcctcactggacttgtccaattaatgaagttgattcatattgcatcatagtttgctttgtttaagcatcacattaaagttaaactgtattttatgttatttatagctgtaggttttctgtgtttagctatttaatactaattttccataagctattttggtttagtgcaaagtataaaattatatttgggggggaataagattatatggactttcttgcaagcaacaagctattttttaaaaaaaactatttaacattcttttgtttatattgttttgtctcctaaattgttgtaattgcattataaaataagaaaaatattaataagacaaatattgaaaataaagaaacaaaaagtt

SEQ ID NO: 16 - DNA, 2917 bp, Homo sapiens
  source 1..2917: mol_type = genomic DNA
gcagacacctgggctgagacatacaggacagagcatggatcgcctacagactgcactcctggttgtcctcgtcctccttgctgtggcgcttcaagcaactgaggcaggcccctacggcgccaacatggaagacagcgtctgctgccgtgattacgtccgttaccgtctgcccctgcgcgtggtgaaacacttctactggacctcagactcctgcccgaggcctggcgtggtgttgctaaccttcagggataaggagatctgtgccgatcccagagtgccctgggtgaagatgattctcaataagctgagccaatgaagagcctactctgatgaccgtggccttggctcctccaggaaggctcaggagccctacctccctgccattatagctgctccccgccagaagcctgtgccaactctctgcattccctgatctccatccctgtggctgtcacccttggtcacctccgtgctgtcactgccatctcccccctgacccctctaacccatcctctgcctccctccctgcagtcagagggtcctgttcccatcagcgattcccctgcttaaacccttccatgactccccactgccctaagctgaggtcagtctcccaagcctggcatgtggccctctggatctgggttccatctctgtctccagcctgcccacttcccttcatgaatgttgggttctagctccctgttctccaaacccatactacacatcccacttctgggtctttgcctgggatgttgctgacacccagaaagtcccaccacctgcacatgtgtagccccaccagccctccaaggcattgctcgcccaagcagctggtaattccatttcatgtattagatgtcccctggccctctgtcccctcttaataaccctagtcacagtctccgcagattcttgggatttgggggttttctcccccacctctccactagttggaccaaggtttctagctaagttactctagtctccaagcctctagcatagagcactgcagacaggccctggctcagaatcagagcccagaaagtggctgcagacaaaatcaataaaactaatgtccctcccctctccctgccaaaaggcagttacatatcaatacagagactcaaggtcactagaaatgggccagctgggtcaatgtgaagccccaaatttgcccagattcacctttcttcccccactccctttttttttttttttttgagatggagtttcgctcttgtcacccacgctggagtgcaatggtgtggtcttggcttattgaagcctctgcctcctgggttcaagtgattctcttgcctcagcctcctgagtagctgggattacaggttcctgctaccacgcccagctaatttttgtatttttagtagagacgaggcttcaccatgttggccaggctggtctcgaactcctgtcctcaggtaatccgcccacctcagcctcccaaagtgctgggattacaggcgtgagccacagtgcctggcctcttccctctccccaccccccccccaacttttttttttttttatggcagggtctcactctgtcgcccaggctggagtgcagtggcgtgatctcggctcactacaacctcgacctcctgggttcaagcgattctcccaccccagcctcccaagtagctgggattacaggtgtgtgccactacggctggctaatttttgtatttttagtagagacaggtttcaccatattggccaggctggtcttgaactcctgacctcaagtgatccaccttccttgtgctcccaaagtgctgagattacaggcgtgagctatcacacccagcctccccctttttttcctaataggagactcctgtacctttcttcgttttacctatgtgtcgtgtctgcttacatttccttctcccctcaggctttttttgggtggtcctccaacctccaatacccaggcctggcctcttcagagtaccccccattccactttccctgcctccttccttaaatagctgacaatcaaattcatgctatggtgtgaaagactacctttgacttggtattataagctggagttatatatgtatttgaaaacagagtaaatacttaagaggccaaatagatgaatggaagaattttaggaactgtgagagggggacaaggtggagctttcctggccctgggaggaagctggctgtggtagcgtagcgctctctctctctgtctgtggcaggaggcaaagagtagggtgtaattgagtgaaggaatcctgggtagagaccattctcaggtggttgggccaggctaaagactgggatttgggtctatctatgcctttctggctgatttttgtagagacggggttttgccatgttacccaggctggtctcaaactcctgggctcaagcgatcctcctggctcagcctcccaaagtgctgggattacaggcgtgagtcactgcgcctggcttcctcttcctcttgagaaatattcttttcatacagcaagtatgggacagcagtgtcccaggtaaaggacataaatgttacaagtgtctggtcctttctgagggaggctggtgccgctctgcagggtatttgaacctgtggaattggaggaggccatttcactccctgaacccagcctgacaaatcacagtgagaatgttcaccttataggcttgctgtggggctcaggttgaaagtgtggggagtgacactgcctaggcatccagctcagtgtcatccagggcctgtgtccctcccgaacccagggtcaacctgcctaccacaggcactagaaggacgaatctgcctactgcccatgaacggggccctcaagcgtcctgggatctccttctccctcctgtcctgtccttgcccctcaggactgctggaaaataaatcctttaaaatag

SEQ ID NO: 17 - DNA, 417 bp, Homo sapiens
  source 1..417: mol_type = genomic DNA
aaggaagagtctaggctgagcaacatgaaggggcccccaaccttctgcagcctcctgctgctgtcattgctcctgagcccagaccctacagcagcattcctactgccacccagcactgcctgctgtactcagctctaccgaaagccactctcagacaagctactgaggaaggtcatccaggtggaactgcaggaggctgacggggactgtcacctccaggctttcgtgcttcacctggctcaacgcagcatctgcatccacccccagaaccccagcctgtcacagtggtttgagcaccaagagagaaagctccatgggactctgcccaagctgaattttgggatgctaaggaaaatgggctgaagcccccaatagccaaataataaagcagcattggataataatttctgagtataa

SEQ ID NO: 18 - DNA, 3660 bp, Homo sapiens
  source 1..3660: mol_type = genomic DNA
gcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtacgttggtgcccgctgctgtctaatgccctggagcctccctggcccccatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaa

SEQ ID NO: 19 - DNA, 1734 bp, Homo sapiens
  source 1..1734: mol_type = genomic DNA
agagtctggaagacctcagaagacctcctgtcctatgaggccctccccatggctttagctgacttgtatgaagaaggaggtggaggaggaggagaaggtgaagacaatgctgactcaaaggagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaacaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatccatcaggccacttgatgacccccaaccaagtggctcccacaccctgttttacaaaaaagaaaagaccagtccatgagggaggtttttaagggtttgtggaaaatgaaaattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctcccccaccgggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgcc

SEQ ID NO: 20 - DNA, 1332 bp, Homo sapiens
  source 1..1332: mol_type = genomic DNA
agaaggaggccaggagttgtgagtttccaagccccagctcactctgaccacttctctgcctgcccagcatcatgaagggccttgcagctgccctccttgtcctcgtctgcaccatggccctctgctcctgtgcacaagttggtaccaacaaagagctctgctgcctcgtctatacctcctggcagattccacaaaagttcatagttgactattctgaaaccagcccccagtgccccaagccaggtgtcatcctcctaaccaagagaggccggcagatctgtgctgaccccaataagaagtgggtccagaaatacatcagcgacctgaagctgaatgcctgaggggcctggaagctgcgagggcccagtgaacttggtgggcccaggagggaacaggagcctgagccagggcaatggccctgccaccctggaggctacctcttctaagagtcccatctgctatgcccagccacattaactaactttaatcttagtttatgcatcatatttcattttgaaattgatttctattgttgagctgcattatgaaattagtattttctctgacatctcatgacattgtctttatcatcctttcccctttcccttcaactcttcgtacattcaatgcatggatcaatcagtgtgattagctttctcagcagacattgtgccatatgtatcaaatgacaaatctttattgaatggttttgctcagcaccaccttttaatatattggcagtacttattatataaaaggtaaaccagcattctcactgtgacgactctgttgattttgtttcactaatcggaatcacagactgagaggaattctgggggaggagtagggaaatatgaaaaggggcagaaacagcaagatagggaccaagtacttctacagtcataccagacatttccctggagatactttcctgaaaagttgaaacagacaccattagtttataaaccatattgtaactgaaatgtgatagaaaaattttctacttaaatgaatatcaagatgacgctgcaatgcatatttacgcacacaagctcattttcataaatgaagctttcctcagaatgatggctaacatctgttgaggtcttaccaagtgctgggttttgggctaagtacttatattagtaaaagtctagcaaatactgcccatagtctagcaaggactccttacctggaagttgctgaaagccttggtaatgttatctttgttctccactctgcttttggggaggatgttttccatgactatacgagatgaggcttggggctgggtggccagagttagcaaataaaaatacagaatggatacta

(19) United States
(12) Patent Application Publication
(10) Pub. No.: US 2023/0242652 A1
(43) Pub. Date: Aug. 3, 2023

(54) TREATMENTS FOR ATOPIC DERMATITIS

(71) Applicants: Galderma Holding S.A., Zug (CH); Chugai Seiyaku Kabushiki Kaisha, Tokyo (JP)

(72) Inventors: Jayendra Kumar KRISHNASWAMY, Pully (CH); Christophe PIKETTY, Montargis (FR)

(73) Assignee: Galderma Holding S.A., Zug (CH)

(21) Appl. No.: 17/897,946

(22) Filed: Aug. 29, 2022

Related U.S. Application Data
(60) Provisional application No. 63/238,643, filed on Aug. 30, 2021.

(51) Int. Cl. C07K 16/28; A61P 17/00

(52) U.S. Cl. CPC C07K 16/2866; A61P 17/00; C07K 2317/565

(57) ABSTRACT

Described herein are treatments and preventions for atopic dermatitis (AD), antibodies and pharmaceutical compositions for use in the treatment or prevention of AD, and uses of an anti-IL-31RA antibody (e.g., nemolizumab) in the manufacture of a medicament for the treatment or prevention of AD. Also described herein are biomarkers of AD and methods of altering or improving these biomarkers via treatments with an antibody that binds to IL-31RA (e.g., nemolizumab).

### SEQUENCE LISTING

This application contains a Sequence Listing which has been submitted electronically in XML file format and is hereby incorporated by reference in its entirety. Said ASCII copy, created Oct. 31, 2022, is named 105153-0411_SL.xml and is 32,808 bytes in size.

### CROSS-REFERENCE TO RELATED APPLICATION

This application claims priority under 35 U.S.C. § 119(e) to U.S. Provisional Application No. 63/238,643, filed Aug. 30, 2021, the entire contents of which are incorporated herein by reference.

### FIELD

Described herein are treatments and preventions for atopic dermatitis (AD), antibodies and pharmaceutical compositions for use in the treatment or prevention of AD, and uses of an anti-IL-31RA antibody (e.g., nemolizumab) in the manufacture of a medicament for the treatment or prevention of AD. Also described herein are biomarkers of AD and methods of altering or improving these biomarkers via treatments with an antibody that binds to IL-31RA (e.g., nemolizumab).

### BACKGROUND

The following discussion is provided to aid the reader in understanding the disclosure and is not admitted to describe or constitute prior art thereto.

Atopic dermatitis (AD) is a chronic inflammatory skin disease characterized by pruritus (itching), xerosis (skin dryness), and eczematous lesions whose features include erythema, infiltration/papulation, oozing with crusting, excoriations, and lichenification. The prevalence of AD is highest in younger children and gradually reduces with age. Prevalence is higher in developed countries.

Although topical treatments are somewhat efficacious, they are not always sufficient to control moderate-to-severe AD in patients, particularly those who therefore require the addition of phototherapy or a systemic treatment to achieve sufficient control of AD. Systemic corticosteroids and other immunosuppressive treatments such as cyclosporine can be effective at controlling disease in some patients; however, given the high response variability and the known secondary adverse effects of these drugs, there is a need for new drugs to better control the disease while decreasing the risk of secondary adverse effects.

There remains a need for treatments for AD and identifying whether patients are likely to respond or are responding to such treatments.

### SUMMARY

Described herein are treatments and preventions for atopic dermatitis (AD) that achieve particular therapeutic results. In general, the treatments and preventions comprise administering to a subject with AD an anti-IL-31RA antibody (e.g., nemolizumab). Also described herein are biomarkers of AD and methods of using the disclosed biomarkers to determine whether a subject is responsive to treatment.

In a first aspect, the present disclosure provides methods of treating or preventing atopic dermatitis (AD) in a subject, comprising administering to a subject with AD an anti-IL-31RA antibody, wherein the subject has skin lesions in which expression of at least one of CCL20, CCL22, CCL27, VEGF, IL1RA, and CCL18 are up-regulated compared to non-lesional skin of an individual without AD or non-lesional skin of the subject.

In a second aspect, the present disclosure provides methods of altering or reducing expression of at least one of CCL20, CCL22, CCL27, VEGF, IL1RA, and CCL18 in a skin lesion of a subject with atopic dermatitis (AD) comprising administering to a subject with AD an anti-IL-31RA antibody, wherein at least one of CCL20, CCL22, CCL27, VEGF, IL1RA, and CCL18 are up-regulated in a skin lesion of the subject compared to non-lesional skin of an individual without AD or non-lesional skin of the subject, and wherein administration of the anti-IL-31RA antibody reduces the expression of at least one of CCL20, CCL22, CCL27, VEGF, IL1RA, and CCL18 in a skin lesion.

In some embodiments of the first and second aspects, CCL20, CCL22, CCL27, and VEGF are up-regulated in the skin lesion of the subject compared to non-lesional skin of an individual without AD or non-lesional skin of the subject.

In some embodiments of the first and second aspects, expression of CCL20, CCL22, CCL27, VEGF, IL1RA, and/or CCL18 is determined by RT-qPCR, RT-PCR, RNA-seq, Northern blotting, Serial Analysis of Gene Expression (SAGE), DNA or RNA microarrays, Western blotting, ELISA, surface plasmon resonance, or mass spectrometry.

In some embodiments of the first and second aspects, one, two, three, four, five, or six of CCL20, CCL22, CCL27, VEGF, IL1RA, and CCL18 are up-regulated in the skin lesion of the subject compared to non-lesional skin of an individual without AD or non-lesional skin of the subject.

In some embodiments of the first and second aspects, the expression of at least one of CCL20, CCL22, CCL27, VEGF, IL1RA, and CCL18 is altered or reduced within 2 weeks, 4 week, 8 weeks, 10 weeks, 12 weeks, 14 weeks, 16 week, 18 weeks, or 20 weeks of the administration of the anti-IL-31RA antibody compared a baseline level of expression in the skin lesion of the subject prior to administration of the anti-IL-31RA antibody.

In some embodiments of the first and second aspects, expression of CCL20 is reduced by at least about 3-fold, expression of CCL22 is reduced by at least about 1.1-fold, expression of CCL27 is reduced by at least about 3-fold, expression of VEGF is reduced by at least about 1.6-fold, expression of CCL18 is reduced by at least about 8-fold, and/or expression of IL1RA is increased by at least about 1.1-fold.

In a third aspect, the present disclosure provides methods of reducing expression of an inflammatory biomarker in the skin of a subject with atopic dermatitis (AD), comprising administering to a subject with AD an anti-IL-31RA antibody, thereby decreasing an inflammatory responses in the skin.

In some embodiments of any of the foregoing aspects, the inflammatory biomarker is selected from at least one of CCL20, CCL22, CCL27, VEGF, IL1RA, and CCL18. In some embodiments of any of the foregoing aspects, the inflammatory biomarker is selected from at least one of CCL20, CCL22, CCL27, and VEGF. In some embodiments of any of the foregoing aspects, expression of one, two, three, or all four of CCL20, CCL22, CCL27, and VEGF is reduced.

In some embodiments of any of the foregoing aspects, expression of the inflammatory biomarker is reduced in at least one skin lesion of the subject.

In some embodiments of any of the foregoing aspects, the subject achieves at least a 66.5% decrease in Eczema Area and Severity Index (EASI) scoring following administration of the anti-IL-31RA antibody. In some embodiments of any of the foregoing aspects, the subject achieves at least a 43.2% decrease in peak pruritis numeric rating scale (PP-NRS) scoring following administration of the anti-IL-31RA antibody. In some embodiments of any of the foregoing aspects, the subject experiences improved sleep following administration of the anti-IL-31RA antibody.

In some embodiments of any of the foregoing aspects, the subject is an adult. In some embodiments of any of the foregoing aspects, the subject is an adolescent, optionally between the ages of 12 and 17 years old.

In some embodiments of any of the foregoing aspects, the anti-IL-31RA antibody is administered subcutaneously.

In some embodiments of any of the foregoing aspects, the anti-IL-31RA antibody is administered once per week, once every two weeks, once every three weeks, once every four weeks, once every five weeks, once every six weeks, once every seven weeks, or once every eight weeks.

2 In some embodiments of any of the foregoing aspects, the anti-IL-31RA antibody is administered at a dose of about 0.01 mg/kg to about 0.1 mg/kg, about 0.1 mg/kg to about 0.5 mg/kg, about 0.5 mg/kg to about 1.5 mg/kg, about 1.5 mg/kg to about 2.5 mg/kg, or about 2.5 mg/kg to about 10 mg/kg.

In some embodiments of any of the foregoing aspects, the anti-IL-31RA antibody is administered at a dose of about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg.

In some embodiments of any of the foregoing aspects, the anti-IL-31RA antibody comprises a heavy chain variable region comprising a HCDR1 comprising SEQ ID NO: 8, a HCDR2 comprising SEQ ID NO: 9, and a HCDR3 comprising SEQ ID NO: 10, and a light chain variable region comprising a LCDR1 comprising SEQ ID NO: 12, a LCDR2 comprising SEQ ID NO: 13, and a LCDR3 comprising SEQ ID NO: 14. In some embodiments of any of the foregoing aspects, the anti-IL-31RA antibody is nemolizumab or a fragment or variant thereof. In some embodiments of any of the foregoing aspects, the anti-IL-31RA antibody is nemolizumab.

In some embodiments of any of the foregoing aspects, the anti-IL-31RA antibody is administered subcutaneously at a loading dose of 60 mg, followed by a dose of 30 mg every four weeks for at least 12, 14, 16, or 18 weeks.

In some embodiments of any of the foregoing aspects, the anti-IL-31RA antibody is administered according to a flat dosing regimen.

In some embodiments of any of the foregoing aspects, the anti-IL-31RA antibody is administered according to a loading dose regimen.

In a fourth aspect, the present disclosure provides methods of diagnosing atopic dermatitis (AD), comprising detecting in a sample obtained from a subject suspected of having AD the expression level of at least one, at least two, at least three, at least four, at least five, or all six biomarker(s) selected from CCL20, CCL22, CCL27, VEGF, IL1RA, and CCL18, and comparing the expression level of the biomarkers to a reference level, wherein the reference level is the corresponding level of expression for each biomarker in a skin sample from an individual that does not have AD or a sample of non-lasional skin from the subject.

In a fifth aspect, the present disclosure provides methods of determining whether a subject with atopic dermatitis (AD) will respond to treatment with an anti-IL-31RA antibody, comprising detecting in a sample obtained from a subject suspected of having AD the expression level of at least one, at least two, at least three, at least four, at least five, or all six biomarker(s) selected from CCL20, CCL22, CCL27, VEGF, IL1RA, and CCL18, and comparing the expression level of the biomarkers to a reference level, wherein the subject will respond to treatment if the expression level of the biomarkers is higher than the reference level, and wherein the reference level is the corresponding level of expression for each biomarker in a skin sample from an individual that does not have AD or a sample of non-lasional skin from the subject.

In some embodiments of the fourth and fifth aspects, the sample obtained from the subject suspected of having AD is a skin sample, which optionally comprises a lesion.

In some embodiments of the fourth and fifth aspects, the expression level of the biomarker(s) is determined by RT-qPCR, RT-PCR, RNA-seq, Northern blotting, Serial Analysis of Gene Expression (SAGE), DNA or RNA microarrays, Western blotting, ELISA, surface plasmon resonance, or mass spectrometry.

In some embodiments of the fourth and fifth aspects, the expression level CCL20 is at least about 1.6-times higher than the reference level, the expression level of CCL22 is at least about 1.02-times higher than the reference level, the expression level of CCL27 is at least about 2.25-times higher than the reference level, expression of VEGF is at least about 1.05-times higher than the reference level, expression of CCL18 is at least about 2.45-times higher than the reference level, and/or expression of IL1RA is increased by at least about 1.1-times higher than the reference level.

In a sixth aspect, the present disclosure provides methods of determining whether a subject with atopic dermatitis (AD) is responding to treatment with an anti-IL-31RA antibody, comprising detecting in a post-treatment sample obtained from a subject with AD that has been administered at least one dose of an anti-IL-31RA antibody the expression level of at least one, at least two, at least three, at least four, at least five, or all six biomarker(s) selected from CCL20, CCL22, CCL27, VEGF, IL1RA, and CCL18, and comparing the expression level of the biomarkers to a baseline level of expression from a sample obtained from the same subject before treatment was commenced, wherein a decrease in the expression level of CCL20, CCL22, CCL27, VEGF, and/or CCL18 and/or an increase in the expression level of IL1RA is indicative of the subject responding to treatment.

In some embodiments of the sixth aspect, the sample is a skin sample, which optionally comprises a lesion.

In some embodiments of the sixth aspect, the expression level of the biomarker(s) is determined by RT-qPCR, RT-PCR, RNA-seq, Northern blotting, Serial Analysis of Gene Expression (SAGE), DNA or RNA microarrays, Western blotting, ELISA, surface plasmon resonance, or mass spectrometry.

In some embodiments of the sixth aspect, the post-treatment expression of CCL20 is reduced by at least about 3-fold, expression of CCL22 is reduced by at least about 1.1-fold, expression of CCL27 is reduced by at least about 3-fold, expression of VEGF is reduced by at least about 1.6-fold, expression of CCL18 is reduced by at least about 8-fold, and/or expression of IL1RA is increased by at least about 1.1-fold lower compared to the baseline level.

In some embodiments of the sixth aspect, the post-treatment sample is obtained about 4 weeks, about 8 weeks, or about 12 weeks after administration of the anti-IL-31RA antibody.

In some embodiments of the sixth aspect, the anti-IL-31RA antibody comprises a heavy chain variable region comprising a HCDR1 comprising SEQ ID NO: 8, a HCDR2 comprising SEQ ID NO: 9, and a HCDR3 comprising SEQ ID NO: 10, and a light chain variable region comprising a LCDR1 comprising SEQ ID NO: 12, a LCDR2 comprising SEQ ID NO: 13, and a LCDR3 comprising SEQ ID NO: 14. In some embodiments of the sixth aspect, the anti-IL-31RA antibody is nemolizumab or a fragment or variant thereof. In some embodiments of the sixth aspect, the anti-IL-31RA antibody is nemolizumab.

In a seventh aspect, the present disclosure provides pharmaceutical compositions comprising an anti-IL-31RA antibody for use in treating or preventing atopic dermatitis (AD) in a subject, wherein the subject has skin lesions in which expression of at least one of CCL20, CCL22, CCL27, VEGF, IL1RA, and CCL18 are up-regulated compared to non-lesional skin of an individual without AD or non-lesional skin of the subject.

In a eighth aspect, the present disclosure provides pharmaceutical compositions comprising an anti-IL-31RA antibody for use in altering or reducing expression of at least one of CCL20, CCL22, CCL27, VEGF, IL1RA, and CCL18 in a skin lesion of a subject with atopic dermatitis (AD) comprising administering to a subject with AD an anti-IL-31RA antibody, wherein at least one of CCL20, CCL22, CCL27, VEGF, IL1RA, and CCL18 are up-regulated in a skin lesion of the subject compared to non-lesional skin of an individual without AD or non-lesional skin of the subject, and wherein administration of the anti-IL-31RA antibody reduces the expression of at least one of CCL20, CCL22, CCL27, VEGF, and CCL18 in a skin lesion and/or increases the expression of IL1RA in a skin lesion.

In a ninth aspect, the present disclosure provides pharmaceutical compositions comprising an anti-IL-31RA antibody for use in reducing expression of an inflammatory biomarker in the skin of a subject with atopic dermatitis (AD).

In some embodiments of the seventh, eight, or ninth aspects, the inflammatory biomarker is selected from at least one of CCL20, CCL22, CCL27, VEGF, IL1RA, and CCL18; preferably CCL20, CCL22, CCL27, and/or VEGF.

In some embodiments of the seventh, eight, or ninth aspects, the subject is an adult.

In some embodiments of the seventh, eight, or ninth aspects, the subject is an adolescent, optionally between the ages of 12 and 17 years old.

In some embodiments of the seventh, eight, or ninth aspects, the pharmaceutical composition is formulated for subcutaneous administration.

In some embodiments of the seventh, eight, or ninth aspects, the anti-IL-31RA antibody comprises a heavy chain variable region comprising a HCDR1 comprising SEQ ID NO: 8, a HCDR2 comprising SEQ ID NO: 9, and a HCDR3 comprising SEQ ID NO: 10, and a light chain variable region comprising a LCDR1 comprising SEQ ID NO: 12, a LCDR2 comprising SEQ ID NO: 13, and a LCDR3 comprising SEQ ID NO: 14. In some embodiments of the seventh, eight, or ninth aspects, the anti-IL-31RA antibody is nemolizumab or a fragment or variant thereof.

In a tenth aspect, the present disclosure provides diagnostic agents for use in diagnosing atopic dermatitis (AD), wherein the diagnostic agent is capable of detecting in a sample obtained from a subject suspected of having AD the expression level of at least one, at least two, at least three, at least four, at least five, or all six biomarker(s) selected from CCL20, CCL22, CCL27, VEGF, IL1RA, and CCL18, such that the expression level of the biomarker(s) can be compared to a reference level, wherein the reference level is the corresponding level of expression for each biomarker in a skin sample from an individual that does not have AD or a sample of non-lasional skin from the subject.

In a eleventh aspect, the present disclosure provides diagnostic agents for use in determining whether a subject with atopic dermatitis (AD) will respond to treatment with an anti-IL-31RA antibody, wherein the diagnostic agent is capable of detecting in a sample obtained from a subject suspected of having AD the expression level of at least one, at least two, at least three, at least four, at least five, or all six biomarker(s) selected from CCL20, CCL22, CCL27, VEGF, IL1RA, and CCL18, such that the expression level of the biomarker(s) can be compared to a reference level, wherein the subject will respond to treatment if the expression level of the biomarker(s) is higher than the reference level, and wherein the reference level is the corresponding level of expression for each biomarker in a skin sample from an individual that does not have AD or a sample of non-lasional skin from the subject.

In a twelfth aspect, the present disclosure provides diagnostic agents for use in determining whether a subject with atopic dermatitis (AD) is responding to treatment with an anti-IL-31RA antibody, wherein the diagnostic agent is capable of detecting in a post-treatment sample obtained from a subject with AD that has been administered at least one dose of an anti-IL-31RA antibody the expression level of at least one, at least two, at least three, at least four, at least five, or all six biomarker(s) selected from CCL20, CCL22, CCL27, VEGF, IL1RA, and CCL18, such that the expression level of the biomarker(s) can be compared to a baseline level of expression from a sample obtained from the same subject before treatment was commenced, wherein a decrease in the expression level of CCL20, CCL22, CCL27, VEGF, and/or CCL18 and/or an increase in the expression level of IL1RA is indicative of the subject responding to treatment. The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF THE DRAWINGS

FIG. 1 shows study design of the clinical trial for treating atopic dermatitis (AD) with nemolizumab. BL=Baseline; n=number of subjects; q4 w=every 4 weeks; TCS=topical corticosteroid; W=week.

FIG. 2 shows mean±standard error (SD) nemolizumab time-concentration profiles (PK population, N=18).

FIG. 3 shows comparison of the popPK simulated and observed nemolizumab concentrations (PK population, N=18) (h=hours; CI=confidence interval; PI=prediction interval). Notes: black solid line is the observed median; black dashed lines are observed 5^{th} and 95^{th} percentiles; blue solid line is the median of the simulated median; red solid lines are the median of the simulated 5^{th} and 95^{th} percentiles. The blue area is the 95% PI of the simulated median, and red areas are the 95% PI of the 5^{th} and 95^{th} percentiles.

FIG. 4 shows population predicted, individual predicted, and observed adolescent eczema area and severity index pharmacokinetic/pharmacodynamic profiles (PK population, N=18). EASI=eczema area and severity index. Notes: Grey dots are observed data points; black solid line is the observed median; black dashed lines are observed 2.5^{th} and 97.5^{th} percentiles. The grey area is the 95% prediction interval (PI) of the simulated median, and blue areas are the 95% PI of the 2.5^{th} and 97.5^{th} percentiles.

FIG. 5 shows population predicted, individual predicted, and observed adolescent peak pruritus numeric rating scale pharmacokinetic/pharmacodynamic profiles (PK population, N=18). PP-NRS=peak pruritus numeric rating scale. Notes: Grey dots are observed data points; black solid line is the observed median; black dashed lines are observed 2.5^{th} and 97.5^{th} percentiles. The grey area is the 95% prediction interval (PI) of the simulated median, and blue areas are the 95% PI of the 2.5^{th} and 97.5^{th} percentiles.

FIG. 6 shows population predicted and observed adolescent Investigator's Global Assessment pharmacokinetic/pharmacodynamic profiles (PK population, N=18). IGA=investigator's global assessment. Notes: Black solid line is the observed median; the grey area is the 95% prediction interval (PI) of the simulated median.

FIG. 7 shows individual subject nemolizumab time-concentration profiles by antidrug antibody result (PK population, N=18).

FIG. 8 shows plot of percent change in Eczema Area and Severity Index (EASI) score over time (last observation carried forward) Intent-to-treat population. Mean±standard error is reported.

FIG. 9 shows plot of proportion of subjects achieving Investigator's Global Assessment success (non-responder) (Intent-to-treat population).

FIG. 10 shows plot of percent change from baseline in weekly average of peak pruritus numeric rating scale score over time (last observation carried forward) (intent-to-treat population). Mean±standard error is reported.

FIG. 11 shows plot of percent change from baseline in weekly average of average pruritus numeric rating scale score over time (last observation carried forward) (intent to treat population). Mean±standard error is reported.

FIG. 12 shows plot of percent change from baseline in weekly average sleep disturbance numeric rating scale score over time (last observation carried forward) (intent-to-treat population). Mean±standard error is reported.

FIG. 13 shows plots of supervised analysis by mixed model repeated measures (MMRM). Note: Supervised analysis (MMRM) of the 6 significantly regulated protein biomarkers in stratum corneum with respect to EASI75. The p-value corresponds to the interaction between sample type and EASI75 responders (Y; blue line) vs non responders (N; red line). Least Square estimated means (LS Means) are shown with 95% confidence intervals for each treatment in each skin condition (lesional/non lesional baseline and week 16). For simplicity, only the interaction p-value is mentioned in the figure, but the selection criterion is based on the minimum adjusted p-value (interaction, main effect).

FIG. 14 shows the results of unsupervised analysis on stratum corneum samples of 15 subjects. Note: Unsupervised analysis (NTF) on stratum corneum samples showing 15 biomarkers that contribute most to the NTF analysis (red=high expression, blue=low expression, X=missing value). The p-value=0.0041 corresponds to the association between the subject response (EASI-75) and the NTF component used for the ranking of the subjects. Y=EASI-75 responders and N=EASI-75 non-responders.

### DETAILED DESCRIPTION

Described herein are treatments and preventions of atopic dermatitis (AD) using an anti-IL-31RA antibody (e.g., nemolizumab), as well as biomarkers and gene signatures associated with AD that were never previously known. The disclosed biomarkers include CCL20, CCL22, CCL27, VEGF, IL1RA, and CCL18. The disclosed treatments and preventions achieve therapeutic endpoints (e.g., decreased Eczema Area and Severity Index (EASI) scoring, decreased peak pruritis numeric rating scale (PP-NRS) scoring, and/or improved sleep) that were not previously known or obtainable with conventional treatments for AD.

### I. Definitions

It is to be understood that the terminology used herein is for the purpose of describing particular embodiments only, and is not intended to be limiting.

Technical and scientific terms used herein have the meanings commonly understood by one of ordinary skill in the art, unless otherwise defined. Unless otherwise specified, materials and/or methodologies known to those of ordinary skill in the art can be utilized in carrying out the methods described herein, based on the guidance provided herein.

As used herein, the singular terms “a,” “an,” and “the” include plural referents unless the context clearly dictates otherwise. Reference to an object in the singular is not intended to mean “one and only one” unless explicitly so stated, but rather “one or more.”

As used herein, “about” when used with a numerical value means the numerical value stated as well as plus or minus 10% of the numerical value. For example, “about 10” should be understood as both “10” and “9-11.”

Also as used herein, “and/or” refers to and encompasses any and all possible combinations of one or more of the associated listed items, as well as the lack of combinations when interpreted in the alternative (“or”).

As used herein, a phrase in the form “A/B” or in the form “A and/or B” means (A), (B), or (A and B); a phrase in the form “at least one of A, B, and C” means (A), (B), (C), (A and B), (A and C), (B and C), or (A, B, and C).

As used herein, the phrase “therapeutically effective amount” with reference to an anti-IL31R antibody (e.g. Nemolizumab) means that dose of the antibody that provides the specific pharmacological effect for which the drug is administered in a subject in need of such treatment. A therapeutically effective amount may be effective to reduce, ameliorate, or eliminate one or more of symptoms/complications of AD including but not limited to pruritus (itching), xerosis (skin dryness), eczematous lesions, and significant sleep disturbances, and/or improve quality of life in a subject with AD. It is emphasized that a therapeutically effective amount of an anti-IL31R antibody (e.g. Nemolizumab) will not always be effective in treating AD in every individual subject, even though such dose is deemed to be a therapeutically effective amount by those of skill in the art. Those skilled in the art can adjust what is deemed to be a therapeutically effective amount in accordance with standard practices as needed to treat a specific subject. A therapeutically effective amount may vary based on, for example, the age and weight of the subject, and/or the subject's overall health, and/or the severity of the subject's AD.

The terms “treat,” “treatment” or “treating” as used herein with reference to AD refer to reducing, ameliorating, or eliminating one or more of symptoms/complications of AD including but not limited to pruritus (itching), xerosis (skin dryness), eczematous lesions, and significant sleep disturbances, and/or improving quality of life in a subject with AD.

The terms “prevent,” “preventing” or “prevention” as used herein with reference to AD refer to precluding or reducing the risk of developing one or more of symptoms/complications of AD including but not limited to pruritus (itching), xerosis (skin dryness), eczematous lesions, and significant sleep disturbances, or preventing the development of the disclosed biomarker signatures that are associated with AD. Prevention may also refer to the prevention of an AD flare or recurrence once an initial flare has been treated or cured.

The terms “individual,” “subject,” and “patient” are used interchangeably herein, and refer to any individual mammalian subject, e.g., bovine, canine, feline, equine, or human. In specific embodiments, the subject, individual, or patient is a human.

### II. Atopic Dermatitis (AD)

Atopic dermatitis (or “AD”) is a skin disease that causes pruritus (itching), xerosis (skin dryness), and/or eczematous lesions whose features include erythema, infiltration/papulation, oozing with crusting, excoriations, and lichenification. Complications of AD may include: asthma, allergic rhinitis (hay fever), chronic itchy or scaly skin, pain, skin infections, irritant hand dermatitis, allergic contact dermatitis, and/or significant sleep disturbances. The prevalence of AD is highest in younger children and gradually reduces with age. Prevalence is higher in developed countries.

The causes of AD are not well understood. The pathophysiology is complex and likely involves a genetic predisposition, epidermal dysfunction, and T-cell driven inflammation. AD has a heritability component and is closely related to and commonly co-occurs with other atopic diseases (such as asthma and allergic rhinitis). Several pathophysiological mechanisms contribute to AD etiology and clinical manifestations. Impairment of epidermal barrier function, for example, owing to deficiency in the structural protein filaggrin, can promote inflammation and T cell infiltration. The immune response in AD is skewed towards T helper 2 cell-mediated pathways and can in turn favor epidermal barrier disruption. Other contributing factors to AD onset include dysbiosis of the skin microbiota (in particular overgrowth of Staphylococcus aureus), systemic immune responses (including immunoglobulin E (IgE)-mediated sensitization) and neuroinflammation, which is involved in itch.

There are no specific diagnostic tests for AD. Diagnosis of the disorder is based on specific criteria that take into account the patient's history and clinical manifestations. Various diagnostic criteria for AD have been proposed and/or validated, which are known to a person of skilled in the art. The Table 1 below provides an exemplary set of diagnostic criteria for AD used in clinic (Williams H C, et al., Br J Dermatol. 131: 383-396 (1994); Williams H C, et al., Br J Dermatol. 131: 397-405 (1994); Williams H C, et al., Br J Dermatol. 131: 406-416 (1994)). Using these criteria, the diagnosis of AD requires the presence of an itchy skin condition (or parental/caregiver reports of scratching or rubbing in a child) plus three or more minor criteria, which vary depending on the patient's age.

TABLE 1
Major criteria
Patient must have
• An itchy skin condition (or parental/caregiver report of
scratching or rubbing in a child)
Minor criteria
Plus three or more of the following minor criteria
Older children/adults
• History of itchiness in skin creases (e.g., folds of elbows,
behind the knees, front of ankles, around the neck)
• Personal history of asthma or allergic rhinitis
• Personal history of general dry skin in the last year
• Visible flexural dermatitis (i.e., in the bends or folds of the
skin at the elbow, knees, wrists, etc.)
• Onset under age 2 years
Children < 4 years^{a}
• History of itching of the cheeks
• History of atopic disease in a first-degree relative
• Eczema of cheeks, forehead and outer limbs
^{a}Early onset not always diagnostic in children under 4 years of age

The clinical manifestations of AD vary with age. In infants, the scalp, face, neck, trunk and extensor (outer) surfaces of the extremities are generally affected, while the diaper area is usually spared. Children typically have involvement of the flexural surfaces of the extremities (i.e., fold/bend at the elbow and back of the knee), neck, wrists and ankles. In adolescence and adulthood, the flexural surfaces of the extremities, hands and feet are usually affected. Regardless of age, the itching associated with AD generally continues throughout the day and worsens at night, leading to sleep loss and substantial impairments in quality of life. It is sometimes difficult to differentiate AD from other skin conditions (e.g., seborrheic dermatitis, contact dermatitis, psoriasis, scabies). In these cases, a family history of atopy and the distribution of lesions may be helpful in making the diagnosis. A punch skin biopsy may be necessary for patients with atypical presentations to rule out other skin conditions that may resemble AD.

Current treatments of AD are directed at restoring the skin barrier, which includes hydrating and repairing the skin, limiting itching, and decreasing inflammation when necessary. Therefore, the successful management of AD requires a multifaceted approach that involves patient and caregiver education, optimal skin care practices, anti-inflammatory treatment with topical corticosteroids (TCSs; first-line) and/or topical calcineurin inhibitors (TCIs), and the treatment of skin infections. Systemic immunosuppressive agents may also be considered in severe cases that cannot be controlled with appropriate skin care and topical therapy. Although first-generation antihistamines are not routinely recommended for the management of AD due to their sedative and impairing side effects, short-term use of these agents may be helpful in those individuals experiencing severe flares of AD, particularly if these flares are associated with significant sleep disturbances. Other therapies that may be beneficial include Ultraviolet (UV) phototherapy, allergen-specific immunotherapy, or wet-wrap therapy (the application of wet bandages over AD lesions after applying emollients and/or topical corticosteroids).

### III. Detection of Biomarkers

The present disclosure is the first to report a biomarker signature for AD that is able to not only identify and positively diagnose AD, but also identify subjects with AD that will likely response to treatment with an anti-IL-31RA antibody (e.g., nemolizumab) and track the response of the treatment with an anti-IL-31RA antibody (e.g., nemolizumab). Polynucleotide or polypeptide biomarkers of the present disclosure include CCL18, CCL20, CCL22, CCL27, CX3CL1, CXCL6, CXCL8, CYSTATIN, FASL, GALECTIN, IL11, IL21, IL1RA, SPD, and VEGF (and more specifically CCL20, CCL22, CCL27, VEGF, IL1RA, and/or CCL18), and these biomarkers may be detected by a variety of methods known in the art. Non-limiting examples of detection methods are described below. The detection assays in the methods of the present disclosure may include purified or isolated DNA (genomic or cDNA), RNA or protein (polypeptide), or the detection step may be performed directly from a biological sample without the need for further DNA, RNA, or protein purification/isolation.

For example, using an immunoassay, it was determined that the cytokines shown in the table below were significantly upregulated in the lesional skin of subjects with AD compared to expression levels in non-lesional skin of the same patient. The quasi-quantitative values shown in the table are a ratio expressing in parts per million (ppm) of the identified cytokine to the total protein content of the sample. As can be seen in this table, expression levels of CCL18, CCL20, CCL22, CCL27, CXCL6, CXCL8, Cystatin, IL1RA, IL11, IL21, SPD, and VEGF were upregulated compared to expression levels in non-lesional skin, while CX3CL1 was down-regulated. Further, expression levels of CCL18, CCL20, CCL22, CCL27, CXCL6, CXCL8, Cystatin, IL21, and VEGF decreased dramatically following a 16 weeks treatment with an anti-IL-31RA antibody (e.g., nemolizumab), while expression levels of CXCL1, IL1RA, IL11, and SPD increased significantly after this course of treatment. Similarly, CCL17 expression was also significantly different between lesional and non-lesional skin in EASI75 responders and was concomitantly reduced post treatment in week 16 lesional skin.

| | | | Percent | | Percent |
|---|---|---|---|---|---|
| | | | differ- | | differ- |
| | | | ence* | | ence** |
| | | | in | | in |
| | | | expression | | expression |
| | | | between | | in lesional |
| | Fraction | Fraction | lesional | Fraction | skin |
| | Base | Base Non- | and non- | W16 | after 16 |
| | Lesional | Lesional | lesional | Lesional | week |
| | (ppm) | (ppm) | skin | (ppm) | treatment |
| CCL18 | 319.85 | 130.43 | 145% | 39.43 | 88% |
| CCL20 | 16.52 | 10.53 | 57% | 5.48 | 67% |
| CCL22 | 1663.32 | 1629.96 | 2% | 1495.42 | 10% |
| CCL27 | 69.91 | 30.94 | 126% | 22.96 | 67% |
| CX3CL1 | 3181.01 | 4458.51 | 29% | 3727.43 | 17% |
| CXCL6 | 43.68 | 27.85 | 57% | 25.55 | 42% |
| CXCL8 | 661.22 | 306.61 | 116% | 248.58 | 62% |
| CYSTATIN | 84351.26 | 68828.98 | 23% | 39914.58 | 53% |
| FASL | 4.48 | 3.38 | 33% | 2.91 | 35% |
| GALECTIN | 2333.92 | 1731.12 | 35% | 962.21 | 59% |
| IL1RA | 229241.75 | 206501.19 | 11% | 255810.65 | 12% |
| IL11 | 15183.41 | 15053.38 | 1% | 18778.70 | 24% |
| IL21 | 6.76 | 5.00 | 35% | 3.43 | 49% |
| SPD | 472.86 | 369.82 | 28% | 764.48 | 62% |
| VEGF | 162.77 | 154.75 | 5% | 100.95 | 38% |

*Percent difference calculated as the absolute value of the difference between the lesional and non-lesional fractions, and divided by the non-lesional fraction (i.e., reference level).
**Percent difference calculated as the absolute value of the difference between the lesional fraction and the lesional fraction after a 16-week treatment (“Fraction W16 Lesional), and divided by the lesional fraction before treatment.

As shown in the table above and the Examples section below, the expression level of the biomarkers may be higher in lesional skin of a subject with AD than the expression levels in non-lesional skin of the subject. Moreover, treatment with an anti-IL-31RA antibody (e.g., nemolizumab) may result in decreases or increases in the expression level of a given biomarker within the lesional skin.

For the purposes of the methods, uses, and compositions disclosed herein, expression of CCL18 in the lesional skin of a subject with AD (or suspected of having AD) may be about 300 ppm (when calculated as a ratio of biomarker protein to total protein in a sample), about 310 ppm, about 320 ppm, about 330 ppm, about 340 ppm, about 350 ppm, about 360 ppm, about 370 ppm, about 380 ppm, about 390 ppm, about 400 ppm, or more. Additionally or alternatively, expression of CCL18 in the lesional skin of a subject with AD (or suspected of having AD) may be at least 300 ppm (when calculated as a ratio of biomarker protein to total protein in a sample), at least 310 ppm, at least 320 ppm, at least 330 ppm, at least 340 ppm, at least 350 ppm, at least 360 ppm, at least 370 ppm, at least 380 ppm, at least 390 ppm, at least 400 ppm, or more. Additionally or alternatively, expression of CCL18 in the lesional skin of a subject with AD (or suspected of having AD) may be about 1.5-fold, about 2-fold, about 2.1-fold, about 2.2-fold, about 2.3-fold, about 2.4-fold, about 2.5-fold, about 2.6-fold, about 2.7-fold, about 2.8-fold, about 2.9-fold, or about 3-fold or more than the expression level of CCL18 in a reference sample (e.g., non-lesional skin of the subject or skin from a subject without AD). Additionally or alternatively, expression of CCL18 in the lesional skin of a subject with AD (or suspected of having AD) may be at least 1.5-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, or at least 3-fold or more than the expression level of CCL18 in a reference sample (e.g., non-lesional skin of the subject or skin from a subject without AD). Additionally or alternatively, expression of CCL18 in the lesional skin of a subject with AD (or suspected of having AD) may be about 150%, about 200%, about 210%, about 220%, about 230%, about 240%, about 250%, about 260%, about 270%, about 280%, about 290%, or about 300% or more of the expression level of CCL18 in a reference sample (e.g., non-lesional skin of the subject or skin from a subject without AD). Additionally or alternatively, expression of CCL18 in the lesional skin of a subject with AD (or suspected of having AD) may be at least 150%, at least 200%, at least 210%, at least 220%, at least 230%, at least 240%, at least 250%, at least 260%, at least 270%, at least 280%, at least 290%, or at least 300% or more of the expression level of CCL18 in a reference sample (e.g., non-lesional skin of the subject or skin from a subject without AD).

For the purposes of the methods, uses, and compositions disclosed herein, treatment with an anti-IL-31RA antibody (e.g., nemolizumab) may decrease expression of CCL18 in the lesional skin of a subject with AD (or suspected of having AD) by about 6-fold, about 6.5-fold, about 7-fold, about 7.5-fold, about 8-fold, about 8.5 fold or more. Treatment duration necessary to achieve such a decrease may be 4 weeks, 6 weeks, 8 weeks, 10 weeks, 12 weeks, 14 weeks, 16 weeks, 18 weeks, 20 weeks, or more. Applicable dosing and administration regimens are discussed in more detail below.

For the purposes of the methods, uses, and compositions disclosed herein, expression of CCL20 in the lesional skin of a subject with AD (or suspected of having AD) may be about 15.5 ppm (when calculated as a ratio of biomarker protein to total protein in a sample), about 15.6 ppm, about 15.7 ppm, about 15.8 ppm, about 15.9 ppm, about 16 ppm, about 16.1 ppm, about 16.2 ppm, about 16.3 ppm, about 16.4 ppm, about 16.5 ppm, about 16.6 ppm, about 16.7 ppm, about 16.8 ppm, about 16.9 ppm, about 17 ppm or more. Additionally or alternatively, expression of CCL20 in the lesional skin of a subject with AD (or suspected of having AD) may be at least 15.5 ppm (when calculated as a ratio of biomarker protein to total protein in a sample), at least 15.6 ppm, at least 15.7 ppm, at least 15.8 ppm, at least 15.9 ppm, at least 16 ppm, at least 16.1 ppm, at least 16.2 ppm, at least 16.3 ppm, at least 16.4 ppm, at least 16.5 ppm, at least 16.6 ppm, at least 16.7 ppm, at least 16.8 ppm, at least 16.9 ppm, at least 17 ppm or more. Additionally or alternatively, expression of CCL20 in the lesional skin of a subject with AD (or suspected of having AD) may be about 1.1-fold, about 1.2-fold, about 1.3-fold, about 1.4-fold, about 1.5-fold, about 1.6-fold, about 1.7-fold, about 1.8-fold, about 1.9-fold, about 2-fold or more than the expression level of CCL20 in a reference sample (e.g., non-lesional skin of the subject or skin from a subject without AD). Additionally or alternatively, expression of CCL20 in the lesional skin of a subject with AD (or suspected of having AD) may be at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold or more than the expression level of CCL20 in a reference sample (e.g., non-lesional skin of the subject or skin from a subject without AD). Additionally or alternatively, expression of CCL20 in the lesional skin of a subject with AD (or suspected of having AD) may be about 110%, about 120%, about 125%, about 130%, about 135%, about 140%, about 145%, about 150%, about 155%, about 160%, about 165%, about 170%, or about 175% or more of the expression level of CCL20 in a reference sample (e.g., non-lesional skin of the subject or skin from a subject without AD). Additionally or alternatively, expression of CCL20 in the lesional skin of a subject with AD (or suspected of having AD) may be at least 110%, at least 120%, at least 125%, at least 130%, at least 135%, at least 140%, at least 145%, at least 150%, at least 155%, at least 160%, at least 165%, at least 170%, or at least 175% or more of the expression level of CCL20 in a reference sample (e.g., non-lesional skin of the subject or skin from a subject without AD).

For the purposes of the methods, uses, and compositions disclosed herein, treatment with an anti-IL-31RA antibody (e.g., nemolizumab) may decrease expression of CCL20 in the lesional skin of a subject with AD (or suspected of having AD) by about 2-fold, about 2.25-fold, about 2.5-fold, about 2.75-fold, about 3-fold, about 3.25-fold, about 3.5-fold or more. Treatment duration necessary to achieve such a decrease may be 4 weeks, 6 weeks, 8 weeks, 10 weeks, 12 weeks, 14 weeks, 16 weeks, 18 weeks, 20 weeks, or more. Applicable dosing and administration regimens are discussed in more detail below.

For the purposes of the methods, uses, and compositions disclosed herein, expression of CCL22 in the lesional skin of a subject with AD (or suspected of having AD) may be about 1550 ppm (when calculated as a ratio of biomarker protein to total protein in a sample), about 1560 ppm, about 1570 ppm, about 1580 ppm, about 1590 ppm, about 1600 ppm, about 1610 ppm, about 1620 ppm, about 1630 ppm, about 1640 ppm, about 1650 ppm, about 1660 ppm, about 1670 ppm, about 1680 ppm, about 1690 ppm, about 1700 ppm or more. Additionally or alternatively, expression of CCL22 in the lesional skin of a subject with AD (or suspected of having AD) may be at least 1550 ppm (when calculated as a ratio of biomarker protein to total protein in a sample), at least 1560 ppm, at least 1570 ppm, at least 1580 ppm, at least 1590 ppm, at least 1600 ppm, at least 1610 ppm, at least 1620 ppm, at least 1630 ppm, at least 1640 ppm, at least 1650 ppm, at least 1660 ppm, at least 1670 ppm, at least 1680 ppm, at least 1690 ppm, at least 1700 ppm or more. Additionally or alternatively, expression of CCL22 in the lesional skin of a subject with AD (or suspected of having AD) may be about 1.01-fold, about 1.02-fold, about 1.03-fold, about 1.04-fold, about 1.05-fold, about 1.06-fold, about 1.07-fold, about 1.08-fold, about 1.09-fold, about 1.1-fold, about 1.2-fold, about 1.3-fold, about 1.4-fold, about 1.5-fold, about 1.6-fold, about 1.7-fold, about 1.8-fold, about 1.9-fold, about 2-fold or more than the expression level of CCL22 in a reference sample (e.g., non-lesional skin of the subject or skin from a subject without AD) or the expression level may be about the same between the lesional skin and the reference sample. Additionally or alternatively, expression of CCL22 in the lesional skin of a subject with AD (or suspected of having AD) may be at least 1.01-fold, at least 1.02-fold, at least 1.03-fold, at least 1.04-fold, at least 1.05-fold, at least 1.06-fold, at least 1.07-fold, at least 1.08-fold, at least 1.09-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold or more than the expression level of CCL22 in a reference sample (e.g., non-lesional skin of the subject or skin from a subject without AD) or the expression level may be about the same between the lesional skin and the reference sample. Additionally or alternatively, expression of CCL22 in the lesional skin of a subject with AD (or suspected of having AD) may be about 101%, about 102%, about 103%, about 104%, about 105%, about 106%, about 107%, about 108%, about 109%, about 110%, about 120%, about 125%, about 130%, about 135%, about 140%, about 145%, about 150%, about 155%, about 160%, about 165%, about 170%, or about 175% or more of the expression level of CCL22 in a reference sample (e.g., non-lesional skin of the subject or skin from a subject without AD). Additionally or alternatively, expression of CCL22 in the lesional skin of a subject with AD (or suspected of having AD) may be at least 101%, at least 102%, at least 103%, at least 104%, at least 105%, at least 106%, at least 107%, at least 108%, at least 109%, at least 110%, at least 120%, at least 125%, at least 130%, at least 135%, at least 140%, at least 145%, at least 150%, at least 155%, at least 160%, at least 165%, at least 170%, or at least 175% or more of the expression level of CCL22 in a reference sample (e.g., non-lesional skin of the subject or skin from a subject without AD).

For the purposes of the methods, uses, and compositions disclosed herein, treatment with an anti-IL-31RA antibody (e.g., nemolizumab) may decrease expression of CCL22 in the lesional skin of a subject with AD (or suspected of having AD) by about 1.1, about 1.2, about 1.3, about 1.4, about 1.5, about 1.6, about 1.7, about 1.8, about 1.9, about 2-fold, about 2.25-fold, about 2.5-fold, about 2.75-fold, about 3-fold, about 3.25-fold, about 3.5-fold or more. Treatment duration necessary to achieve such a decrease may be 4 weeks, 6 weeks, 8 weeks, 10 weeks, 12 weeks, 14 weeks, 16 weeks, 18 weeks, 20 weeks, or more. Applicable dosing and administration regimens are discussed in more detail below.

For the purposes of the methods, uses, and compositions disclosed herein, expression of CCL27 in the lesional skin of a subject with AD (or suspected of having AD) may be about 50 ppm (when calculated as a ratio of biomarker protein to total protein in a sample), about 51 ppm, about 52 ppm, about 53 ppm, about 54 ppm, about 55 ppm, about 56 ppm, about 57 ppm, about 58 ppm, about 59 ppm, about 60 ppm, about 61 ppm, about 62 ppm, about 63 ppm, about 64 ppm, about 65 ppm, about 66 ppm, about 67 ppm, about 68 ppm, about 69 ppm, about 70 ppm, about 71 ppm, about 72 ppm, about 73 ppm, about 74 ppm, about 75 ppm or more. Additionally or alternatively, expression of CCL27 in the lesional skin of a subject with AD (or suspected of having AD) may be at least 50 ppm (when calculated as a ratio of biomarker protein to total protein in a sample), at least 51 ppm, at least 52 ppm, at least 53 ppm, at least 54 ppm, at least 55 ppm, at least 56 ppm, at least 57 ppm, at least 58 ppm, at least 59 ppm, at least 60 ppm, at least 61 ppm, at least 62 ppm, at least 63 ppm, at least 64 ppm, at least 65 ppm, at least 66 ppm, at least 67 ppm, at least 68 ppm, at least 69 ppm, at least 70 ppm, at least 71 ppm, at least 72 ppm, at least 73 ppm, at least 74 ppm, at least 75 ppm or more. Additionally or alternatively, expression of CCL27 in the lesional skin of a subject with AD (or suspected of having AD) may be about 1.5-fold, about 2-fold, about 2.1-fold, about 2.2-fold, about 2.3-fold, about 2.4-fold, about 2.5-fold, about 2.6-fold, about 2.7-fold, about 2.8-fold, about 2.9-fold, or about 3-fold or more than the expression level of CCL27 in a reference sample (e.g., non-lesional skin of the subject or skin from a subject without AD). Additionally or alternatively, expression of CCL27 in the lesional skin of a subject with AD (or suspected of having AD) may be at least 1.5-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, or at least 3-fold or more than the expression level of CCL27 in a reference sample (e.g., non-lesional skin of the subject or skin from a subject without AD). Additionally or alternatively, expression of CCL27 in the lesional skin of a subject with AD (or suspected of having AD) may be about 150%, about 175%, about 200%, about 210%, about 220%, about 225% about 230%, about 240%, about 250%, about 260%, about 270%, about 280%, about 290%, or about 300% or more of the expression level of CCL27 in a reference sample (e.g., non-lesional skin of the subject or skin from a subject without AD). Additionally or alternatively, expression of CCL27 in the lesional skin of a subject with AD (or suspected of having AD) may be at least 150%, at least 175%, at least 200%, at least 210%, at least 220%, at least 225%, at least 230%, at least 240%, at least 250%, at least 260%, at least 270%, at least 280%, at least 290%, or at least 300% or more of the expression level of CCL27 in a reference sample (e.g., non-lesional skin of the subject or skin from a subject without AD).

For the purposes of the methods, uses, and compositions disclosed herein, treatment with an anti-IL-31RA antibody (e.g., nemolizumab) may decrease expression of CCL27 in the lesional skin of a subject with AD (or suspected of having AD) by about 1.5-fold, about 1.75-fold, about 2-fold, about 2.25-fold, about 2.5-fold, about 2.75 fold, about 3-fold, about 3.25-fold, about 3.5-fold or more. Treatment duration necessary to achieve such a decrease may be 4 weeks, 6 weeks, 8 weeks, 10 weeks, 12 weeks, 14 weeks, 16 weeks, 18 weeks, 20 weeks, or more. Applicable dosing and administration regimens are discussed in more detail below.

For the purposes of the methods, uses, and compositions disclosed herein, expression of VEGF in the lesional skin of a subject with AD (or suspected of having AD) may be about 150 ppm (when calculated as a ratio of biomarker protein to total protein in a sample), about 151 ppm, about 152 ppm, about 153 ppm, about 154 ppm, about 155 ppm, about 156 ppm, about 157 ppm, about 158 ppm, about 159 ppm, about 160 ppm, about 161 ppm, about 162 ppm, about 163 ppm, about 164 ppm, about 165 ppm, about 166 ppm, about 167 ppm, about 168 ppm, about 169 ppm, about 170 ppm, about 171 ppm, about 172 ppm, about 173 ppm, about 174 ppm, about 175 ppm or more. Additionally or alternatively, expression of CCL27 in the lesional skin of a subject with AD (or suspected of having AD) may be at least 150 ppm (when calculated as a ratio of biomarker protein to total protein in a sample), at least 151 ppm, at least 152 ppm, at least 153 ppm, at least 154 ppm, at least 155 ppm, at least 156 ppm, at least 157 ppm, at least 158 ppm, at least 159 ppm, at least 160 ppm, at least 161 ppm, at least 162 ppm, at least 163 ppm, at least 164 ppm, at least 165 ppm, at least 166 ppm, at least 167 ppm, at least 168 ppm, at least 169 ppm, at least 170 ppm, at least 171 ppm, at least 172 ppm, at least 173 ppm, at least 174 ppm, at least 75 ppm or more. Additionally or alternatively, expression of VEGF in the lesional skin of a subject with AD (or suspected of having AD) may be about 1.01-fold, about 1.02-fold, about 1.03-fold, about 1.04-fold, about 1.05-fold, about 1.06-fold, about 1.07-fold, about 1.08-fold, about 1.09-fold, about 1.1-fold, about 1.2-fold, about 1.3-fold, about 1.4-fold, about 1.5-fold, about 1.6-fold, about 1.7-fold, about 1.8-fold, about 1.9-fold, about 2-fold or more than the expression level of VEGF in a reference sample (e.g., non-lesional skin of the subject or skin from a subject without AD) or the expression level may be about the same between the lesional skin and the reference sample. Additionally or alternatively, expression of VEGF in the lesional skin of a subject with AD (or suspected of having AD) may be at least 1.01-fold, at least 1.02-fold, at least 1.03-fold, at least 1.04-fold, at least 1.05-fold, at least 1.06-fold, at least 1.07-fold, at least 1.08-fold, at least 1.09-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold or more than the expression level of VEGF in a reference sample (e.g., non-lesional skin of the subject or skin from a subject without AD) or the expression level may be about the same between the lesional skin and the reference sample. Additionally or alternatively, expression of VEGF in the lesional skin of a subject with AD (or suspected of having AD) may be about 101%, about 102%, about 103%, about 104%, about 105%, about 106%, about 107%, about 108%, about 109%, about 110%, about 120%, about 125%, about 130%, about 135%, about 140%, about 145%, about 150%, about 155%, about 160%, about 165%, about 170%, or about 175% or more of the expression level of VEGF in a reference sample (e.g., non-lesional skin of the subject or skin from a subject without AD). Additionally or alternatively, expression of VEGF in the lesional skin of a subject with AD (or suspected of having AD) may be at least 101%, at least 102%, at least 103%, at least 104%, at least 105%, at least 106%, at least 107%, at least 108%, at least 109%, at least 110%, at least 120%, at least 125%, at least 130%, at least 135%, at least 140%, at least 145%, at least 150%, at least 155%, at least 160%, at least 165%, at least 170%, or at least 175% or more of the expression level of VEGF in a reference sample (e.g., non-lesional skin of the subject or skin from a subject without AD).

For the purposes of the methods, uses, and compositions disclosed herein, treatment with an anti-IL-31RA antibody (e.g., nemolizumab) may decrease expression of VEGF in the lesional skin of a subject with AD (or suspected of having AD) by about 1.1, about 1.2, about 1.3, about 1.4, about 1.5, about 1.6, about 1.7, about 1.8, about 1.9, about 2-fold, about 2.25-fold, about 2.5-fold, about 2.75-fold, about 3-fold, about 3.25-fold, about 3.5-fold or more. Treatment duration necessary to achieve such a decrease may be 4 weeks, 6 weeks, 8 weeks, 10 weeks, 12 weeks, 14 weeks, 16 weeks, 18 weeks, 20 weeks, or more. Applicable dosing and administration regimens are discussed in more detail below.

For the purposes of the methods, uses, and compositions disclosed herein, expression of IL1RA in the lesional skin of a subject with AD (or suspected of having AD) may be about 207000 ppm (when calculated as a ratio of biomarker protein to total protein in a sample), about 207500 ppm, about 208000 ppm, about 208500 ppm, about 209000 ppm, about 209500 ppm, about 210000 ppm, about 210500 ppm, about 211000 ppm, about 211500 ppm, about 212000 ppm, about 212500 ppm, about 213000 ppm, about 213500 ppm, about 214000 ppm, about 214500 ppm, about 215000 ppm, about 215500 ppm, about 216000 ppm, about 216500 ppm, about 217000 ppm, about 217500 ppm, about 218000 ppm, about 218500 ppm, about 219000 ppm, about 219500, about 220000, about 221000, about 222000, about 223000, about 224000, about 225000, about 226000, about 227000, about 228000, about 229000, about 230000, about 231000, about 232000, about 233000, about 234000, about 235000 ppm or more. Additionally or alternatively, expression of CCL27 in the lesional skin of a subject with AD (or suspected of having AD) may be at least 207000 ppm (when calculated as a ratio of biomarker protein to total protein in a sample), at least 207500 ppm, at least 208000 ppm, at least 208500 at least, at least 209000 ppm, at least 209500 ppm, at least 210000 ppm, at least 210500 ppm, at least 211000 ppm, at least 211500 ppm, at least 212000 ppm, at least 212500 ppm, at least 213000 ppm, at least 213500 ppm, at least 214000 ppm, at least 214500 ppm, at least 215000 ppm, at least 215500 ppm, at least 216000 ppm, at least 216500 ppm, at least 217000 ppm, at least 217500 ppm, at least 218000 ppm, at least 218500 ppm, at least 219000 ppm, at least 219500, at least 220000, at least 221000, at least 222000, at least 223000, at least 224000, at least 225000, at least 226000, at least 227000, at least 228000, at least 229000, at least 230000, at least 231000, at least 232000, at least 233000, at least 234000, at least 235000 ppm or more. Additionally or alternatively, expression of IL1RA in the lesional skin of a subject with AD (or suspected of having AD) may be about 1.01-fold, about 1.02-fold, about 1.03-fold, about 1.04-fold, about 1.05-fold, about 1.06-fold, about 1.07-fold, about 1.08-fold, about 1.09-fold, about 1.1-fold, about 1.2-fold, about 1.3-fold, about 1.4-fold, about 1.5-fold, about 1.6-fold, about 1.7-fold, about 1.8-fold, about 1.9-fold, about 2-fold or more than the expression level of IL1RA in a reference sample (e.g., non-lesional skin of the subject or skin from a subject without AD) or the expression level may be about the same between the lesional skin and the reference sample. Additionally or alternatively, expression of IL1RA in the lesional skin of a subject with AD (or suspected of having AD) may be at least 1.01-fold, at least 1.02-fold, at least 1.03-fold, at least 1.04-fold, at least 1.05-fold, at least 1.06-fold, at least 1.07-fold, at least 1.08-fold, at least 1.09-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold or more than the expression level of IL1RA in a reference sample (e.g., non-lesional skin of the subject or skin from a subject without AD) or the expression level may be about the same between the lesional skin and the reference sample. Additionally or alternatively, expression of IL1RA in the lesional skin of a subject with AD (or suspected of having AD) may be about 101%, about 102%, about 103%, about 104%, about 105%, about 106%, about 107%, about 108%, about 109%, about 110%, about 115%, about 120%, about 125%, about 130%, about 135%, about 140%, about 145%, about 150%, about 155%, about 160%, about 165%, about 170%, or about 175% or more of the expression level of IL1RA in a reference sample (e.g., non-lesional skin of the subject or skin from a subject without AD). Additionally or alternatively, expression of IL1RA in the lesional skin of a subject with AD (or suspected of having AD) may be at least 101%, at least 102%, at least 103%, at least 104%, at least 105%, at least 106%, at least 107%, at least 108%, at least 109%, at least 110%, at least 115%, at least 120%, at least 125%, at least 130%, at least 135%, at least 140%, at least 145%, at least 150%, at least 155%, at least 160%, at least 165%, at least 170%, or at least 175% or more of the expression level of IL1RA in a reference sample (e.g., non-lesional skin of the subject or skin from a subject without AD).

For the purposes of the methods, uses, and compositions disclosed herein, treatment with an anti-IL-31RA antibody (e.g., nemolizumab) may increase expression of IL1RA in the lesional skin of a subject with AD (or suspected of having AD) by about 1.1, about 1.2, about 1.3, about 1.4, about 1.5, about 1.6, about 1.7, about 1.8, about 1.9, about 2-fold, about 2.25-fold, about 2.5-fold, about 2.75-fold, about 3-fold, about 3.25-fold, about 3.5-fold or more. Treatment duration necessary to achieve such a decrease may be 4 weeks, 6 weeks, 8 weeks, 10 weeks, 12 weeks, 14 weeks, 16 weeks, 18 weeks, 20 weeks, or more. Applicable dosing and administration regimens are discussed in more detail below.

For the purposes of the methods, uses, and compositions disclosed herein, the other noted biomarkers (e.g., CXCL6, CXCL8, Cystatin, FASL, Galectin, IL11, IL21, SPD, and CCL17) may show similar patterns of overexpression in the lesional skin of a subject with AD (or suspected of having AD) compared to expression levels in a reference sample (e.g., non-lesional skin of the subject or skin from a subject without AD), and CX3CL1 may show decreased expression in the lesional skin of a subject with AD (or suspected of having AD) compared to expression levels in a reference sample (e.g., non-lesional skin of the subject or skin from a subject without AD). Similarly, CXCL6, CXCL8, Cystatin, FASL, Galectin, CCL17, and IL21 may show similar patterns of decreases of expression in the lesional skin of a subject with AD (or suspected of having AD) after treatment with an anti-IL-31RA antibody (e.g., nemolizumab), and CX3CL1, IL11, and SPD may show increases in expression in the lesional skin of a subject with AD (or suspected of having AD) after treatment with an anti-IL-31RA antibody (e.g., nemolizumab).

In some embodiments, protein or polypeptide expression levels of the disclosed biomarkers (e.g., CCL20, CCL22, CCL27, VEGF, IL1RA, and/or CCL18) may be detected via Western blotting, enzyme-linked immunosorbent assays (ELISA), dot blotting, immunohistochemistry, immunofluorescence, immunoprecipitation, immunoelectrophoresis, or mass-spectrometry.

Additionally or alternatively, in some embodiments, polynucleotides encoding the disclosed biomarkers (e.g., CCL20, CCL22, CCL27, VEGF, IL1RA, and/or CCL18) may be detected by RT-qPCR, RT-PCR, RNA-seq, Northern blotting, Serial Analysis of Gene Expression (SAGE), or DNA or RNA microarrays. The starting material for detection of polynucleotides encoding the disclosed biomarkers may be genomic DNA, cDNA, RNA or mRNA. Nucleic acid amplification can be linear or exponential. Specific variants or mutations may be detected by the use of amplification methods with the aid of oligonucleotide primers or probes designed to interact with or hybridize to a particular target sequence in a specific manner, thus amplifying only the target variant. Primers and probes may also include a detectable label or a plurality of detectable labels. The detectable label associated with the probe can generate a detectable signal directly. Additionally, the detectable label associated with the probe can be detected indirectly using a reagent, wherein the reagent includes a detectable label, and binds to the label associated with the probe.

In some embodiments, detectably labeled primers or probes can be used in hybridization assays including, but not limited to Northern blots, Southern blots, microarray, dot or slot blots, and in situ hybridization assays such as fluorescent in situ hybridization (FISH) to detect a target nucleic acid sequence within a biological sample. Detectably labeled probes can also be used to monitor the amplification of a target nucleic acid sequence. In some embodiments, detectably labeled probes present in an amplification reaction are suitable for monitoring the amount of amplicon(s) produced as a function of time. Examples of such probes include, but are not limited to, the 5′-exonuclease assay (TAQMAN® probes described herein (see also U.S. Pat. No. 5,538,848) various stem-loop molecular beacons (see for example, U.S. Pat. Nos. 6,103,476 and 5,925,517 and Tyagi and Kramer, 1996, Nature Biotechnology 14:303-308), stemless or linear beacons (see, e.g., WO 99/21881), PNA Molecular Beacons™ (see, e.g., U.S. Pat. Nos. 6,355,421 and 6,593,091), linear PNA beacons (see, for example, Kubista et al., 2001, SPIE 4264:53-58), non-FRET probes (see, for example, U.S. Pat. No. 6,150,097), Sunrise®/Amplifluor™ probes (U.S. Pat. No. 6,548,250), stem-loop and duplex Scorpion probes (Solinas et al., 2001, Nucleic Acids Research 29:E96 and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse™ probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem. Res. Toxicol. 15:118-126; and Yu et al., 2001, J. Am. Chem. Soc 14:11155-11161. In some embodiments, the detectable label is a fluorophore. Suitable fluorescent moieties include but are not limited to the following fluorophores working individually or in combination: 4-acetamido-4′-isothiocyanatostilbene-2,2′disulfonic acid; acridine and derivatives: acridine, acridine isothiocyanate; Alexa Fluors: Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Black Hole Quencher™ (BHQ™) dyes (biosearch Technologies); BODIPY dyes: BODIPY® R-6G, BOPIPY® 530/550, BODIPY® FL; Brilliant Yellow; coumarin and derivatives: coumarin, 7-amino-4-methylcoumarin (AMC, Coumarin 120), 7-amino-4-trifluoromethylcouluarin (Coumarin 151); Cy2®, Cy3®, Cy3.5@, Cy5®, Cy5.5@; cyanosine; 4′,6-diaminidino-2-phenylindole (DAPI); 5′, 5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); Eclipse™ (Epoch Biosciences Inc.); eosin and derivatives: eosin, eosin isothiocyanate; erythrosin and derivatives: erythrosin B, erythrosin isothiocyanate; ethidium; fluorescein and derivatives: 5-carboxyfluorescein (FAM), 5-(4,6-dichlorotriazin-2-yl)amino fluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescem (TET); fluorescamine; IR144; IR1446; lanthamide phosphors; Malachite Green isothiocyanate; 4-methylumbelliferone; ortho cresolphthalein; nitrotyrosine; pararosaniline; Phenol Red; B-phycoerythrin, R-phycoerythrin; allophycocyanin; o-phthaldialdehyde; Oregon Green®; propidium iodide; pyrene and derivatives: pyrene, pyrene butyrate, succinimidyl 1-pyrene butyrate; QSY® 7; QSY® 9; QSY® 21; QSY® 35 (Molecular Probes); Reactive Red 4 (Cibacron®Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, riboflavin, rosolic acid, sulforhodamine B, sulforhodamine 101, sulfonyl chloride derivative of sulforhodamine 101 (Texas Red); terbium chelate derivatives; N,N,N′,N′-tetramethyl-6-carboxyrhodamine (TAMRA); tetramethyl rhodamine; tetramethyl rhodamine isothiocyanate (TRITC); and VIC®. Detector probes can also comprise sulfonate derivatives of fluorescein dyes with S03 instead of the carboxylate group, phosphoramidite forms of fluorescein, phosphoramidite forms of CY 5 (commercially available for example from Amersham).

Primers or probes may be designed to selectively hybridize to any portion of a nucleic acid sequence encoding a polypeptide biomarkers of the present disclosure (e.g., CCL20, CCL22, CCL27, VEGF, IL1RA, and/or CCL18). Methods for preparing the primers or probes have been well developed in the art. Exemplary nucleic acid sequences of the human orthologs of these genes are provided below:

```
Homo sapiens C-C motif chemokine ligand 20 (CCL20), transcript variant 1, mRNA
(NCBI Reference Sequence: NM_004591.3)
(SEQ ID NO: 15)
      1 agaatataac agcactccca aagaactggg tactcaacac tgagcagatc tgttctttga
    61 gctaaaaacc atgtgctgta ccaagagttt gctcctggct gctttgatgt cagtgctgct
  121 actccacctc tgcggcgaat cagaagcagc aagcaacttt gactgctgtc ttggatacac
  181 agaccgtatt cttcatccta aatttattgt gggcttcaca cggcagctgg ccaatgaagg
  241 ctgtgacatc aatgctatca tctttcacac aaagaaaaag ttgtctgtgt gcgcaaatcc
  301 aaaacagact tgggtgaaat atattgtgcg tctcctcagt aaaaaagtca agaacatgta
  361 aaaactgtgg cttttctgga atggaattgg acatagccca agaacagaaa gaaccttgct
  421 ggggttggag gtttcacttg cacatcatgg agggtttagt gcttatctaa tttgtgcctc
  481 actggacttg tccaattaat gaagttgatt catattgcat catagtttgc tttgtttaag
  541 catcacatta aagttaaact gtattttatg ttatttatag ctgtaggttt tctgtgttta
  601 gctatttaat actaattttc cataagctat tttggtttag tgcaaagtat aaaattatat
  661 ttggggggga ataagattat atggactttc ttgcaagcaa caagctattt tttaaaaaaa
  721 actatttaac attcttttgt ttatattgtt ttgtctccta aattgttgta attgcattat
  781 aaaataagaa aaatattaat aagacaaata ttgaaaataa agaaacaaaa agtt
Homo sapiens C-C motif chemokine ligand 22 (CCL22), mRNA, (NCBI Reference
Sequence: NM_002990.5)
(SEQ ID NO: 16)
      1 gcagacacct gggctgagac atacaggaca gagcatggat cgcctacaga ctgcactcct
    61 ggttgtcctc gtcctccttg ctgtggcgct tcaagcaact gaggcaggcc cctacggcgc
  121 caacatggaa gacagcgtct gctgccgtga ttacgtccgt taccgtctgc ccctgcgcgt
  181 ggtgaaacac ttctactgga cctcagactc ctgcccgagg cctggcgtgg tgttgctaac
  241 cttcagggat aaggagatct gtgccgatcc cagagtgccc tgggtgaaga tgattctcaa
  301 taagctgagc caatgaagag cctactctga tgaccgtggc cttggctcct ccaggaaggc
  361 tcaggagccc tacctccctg ccattatagc tgctccccgc cagaagcctg tgccaactct
  421 ctgcattccc tgatctccat ccctgtggct gtcacccttg gtcacctccg tgctgtcact
  481 gccatctccc ccctgacccc tctaacccat cctctgcctc cctccctgca gtcagagggt
  541 cctgttccca tcagcgattc ccctgcttaa acccttccat gactccccac tgccctaagc
  601 tgaggtcagt ctcccaagcc tggcatgtgg ccctctggat ctgggttcca tctctgtctc
  661 cagcctgccc acttcccttc atgaatgttg ggttctagct ccctgttctc caaacccata
  721 ctacacatcc cacttctggg tctttgcctg ggatgttgct gacacccaga aagtcccacc
  781 acctgcacat gtgtagcccc accagccctc caaggcattg ctcgcccaag cagctggtaa
  841 ttccatttca tgtattagat gtcccctggc cctctgtccc ctcttaataa ccctagtcac
  901 agtctccgca gattcttggg atttgggggt tttctccccc acctctccac tagttggacc
  961 aaggtttcta gctaagttac tctagtctcc aagcctctag catagagcac tgcagacagg
1021 ccctggctca gaatcagagc ccagaaagtg gctgcagaca aaatcaataa aactaatgtc
1081 cctcccctct ccctgccaaa aggcagttac atatcaatac agagactcaa ggtcactaga
1141 aatgggccag ctgggtcaat gtgaagcccc aaatttgccc agattcacct ttcttccccc
1201 actccctttt tttttttttt tttgagatgg agtttcgctc ttgtcaccca cgctggagtg
1261 caatggtgtg gtcttggctt attgaagcct ctgcctcctg ggttcaagtg attctcttgc
1321 ctcagcctcc tgagtagctg ggattacagg ttcctgctac cacgcccagc taatttttgt
1381 atttttagta gagacgaggc ttcaccatgt tggccaggct ggtctcgaac tcctgtcctc
1441 aggtaatccg cccacctcag cctcccaaag tgctgggatt acaggcgtga gccacagtgc
1501 ctggcctctt ccctctcccc accccccccc caactttttt ttttttttat ggcagggtct
1561 cactctgtcg cccaggctgg agtgcagtgg cgtgatctcg gctcactaca acctcgacct
1621 cctgggttca agcgattctc ccaccccagc ctcccaagta gctgggatta caggtgtgtg
1681 ccactacggc tggctaattt ttgtattttt agtagagaca ggtttcacca tattggccag
1741 gctggtcttg aactcctgac ctcaagtgat ccaccttcct tgtgctccca aagtgctgag
1801 attacaggcg tgagctatca cacccagcct cccccttttt ttcctaatag gagactcctg
1861 tacctttctt cgttttacct atgtgtcgtg tctgcttaca tttccttctc ccctcaggct
1921 ttttttgggt ggtcctccaa cctccaatac ccaggcctgg cctcttcaga gtacccccca
1981 ttccactttc cctgcctcct tccttaaata gctgacaatc aaattcatgc tatggtgtga
2041 aagactacct ttgacttggt attataagct ggagttatat atgtatttga aaacagagta
2101 aatacttaag aggccaaata gatgaatgga agaattttag gaactgtgag agggggacaa
2161 ggtggagctt tcctggccct gggaggaagc tggctgtggt agcgtagcgc tctctctctc
2221 tgtctgtggc aggaggcaaa gagtagggtg taattgagtg aaggaatcct gggtagagac
2281 cattctcagg tggttgggcc aggctaaaga ctgggatttg ggtctatcta tgcctttctg
2341 gctgattttt gtagagacgg ggttttgcca tgttacccag gctggtctca aactcctggg
2401 ctcaagcgat cctcctggct cagcctccca aagtgctggg attacaggcg tgagtcactg
2461 cgcctggctt cctcttcctc ttgagaaata ttcttttcat acagcaagta tgggacagca
2521 gtgtcccagg taaaggacat aaatgttaca agtgtctggt cctttctgag ggaggctggt
2581 gccgctctgc agggtatttg aacctgtgga attggaggag gccatttcac tccctgaacc
2641 cagcctgaca aatcacagtg agaatgttca ccttataggc ttgctgtggg gctcaggttg
2701 aaagtgtggg gagtgacact gcctaggcat ccagctcagt gtcatccagg gcctgtgtcc
2761 ctcccgaacc cagggtcaac ctgcctacca caggcactag aaggacgaat ctgcctactg
2821 cccatgaacg gggccctcaa gcgtcctggg atctccttct ccctcctgtc ctgtccttgc
2881 ccctcaggac tgctggaaaa taaatccttt aaaatag
Homo sapiens C-C motif chemokine ligand 27 (CCL27), mRNA (NCBI Reference
Sequence: NM_006664.4)
(SEQ ID NO: 17)
      1 aaggaagagt ctaggctgag caacatgaag gggcccccaa ccttctgcag cctcctgctg
    61 ctgtcattgc tcctgagccc agaccctaca gcagcattcc tactgccacc cagcactgcc
  121 tgctgtactc agctctaccg aaagccactc tcagacaagc tactgaggaa ggtcatccag
  181 gtggaactgc aggaggctga cggggactgt cacctccagg ctttcgtgct tcacctggct
  241 caacgcagca tctgcatcca cccccagaac cccagcctgt cacagtggtt tgagcaccaa
  301 gagagaaagc tccatgggac tctgcccaag ctgaattttg ggatgctaag gaaaatgggc
  361 tgaagccccc aatagccaaa taataaagca gcattggata ataatttctg agtataa
Homo sapiens vascular endothelial growth factor A (VEGFA), transcript variant 1,
mRNA (NCBI Reference Sequence: NM_001025366.3)
(SEQ ID NO: 18)
      1 gcggaggctt ggggcagccg ggtagctcgg aggtcgtggc gctgggggct agcaccagcg
    61 ctctgtcggg aggcgcagcg gttaggtgga ccggtcagcg gactcaccgg ccagggcgct
  121 cggtgctgga atttgatatt cattgatccg ggttttatcc ctcttctttt ttcttaaaca
  181 ttttttttta aaactgtatt gtttctcgtt ttaatttatt tttgcttgcc attccccact
  241 tgaatcgggc cgacggcttg gggagattgc tctacttccc caaatcactg tggattttgg
  301 aaaccagcag aaagaggaaa gaggtagcaa gagctccaga gagaagtcga ggaagagaga
  361 gacggggtca gagagagcgc gcgggcgtgc gagcagcgaa agcgacaggg gcaaagtgag
  421 tgacctgctt ttgggggtga ccgccggagc gcggcgtgag ccctccccct tgggatcccg
  481 cagctgacca gtcgcgctga cggacagaca gacagacacc gcccccagcc ccagctacca
  541 cctcctcccc ggccggcggc ggacagtgga cgcggcggcg agccgcgggc aggggccgga
  601 gcccgcgccc ggaggcgggg tggagggggt cggggctcgc ggcgtcgcac tgaaactttt
  661 cgtccaactt ctgggctgtt ctcgcttcgg aggagccgtg gtccgcgcgg gggaagccga
  721 gccgagcgga gccgcgagaa gtgctagctc gggccgggag gagccgcagc cggaggaggg
  781 ggaggaggaa gaagagaagg aagaggagag ggggccgcag tggcgactcg gcgctcggaa
  841 gccgggctca tggacgggtg aggcggcggt gtgcgcagac agtgctccag ccgcgcgcgc
  901 tccccaggcc ctggcccggg cctcgggccg gggaggaaga gtagctcgcc gaggcgccga
  961 ggagagcggg ccgccccaca gcccgagccg gagagggagc gcgagccgcg ccggccccgg
1021 tcgggcctcc gaaaccatga actttctgct gtcttgggtg cattggagcc ttgccttgct
1081 gctctacctc caccatgcca agtggtccca ggctgcaccc atggcagaag gaggagggca
1141 gaatcatcac gaagtggtga agttcatgga tgtctatcag cgcagctact gccatccaat
1201 cgagaccctg gtggacatct tccaggagta ccctgatgag atcgagtaca tcttcaagcc
1261 atcctgtgtg cccctgatgc gatgcggggg ctgctgcaat gacgagggcc tggagtgtgt
1321 gcccactgag gagtccaaca tcaccatgca gattatgcgg atcaaacctc accaaggcca
1381 gcacatagga gagatgagct tcctacagca caacaaatgt gaatgcagac caaagaaaga
1441 tagagcaaga caagaaaaaa aatcagttcg aggaaaggga aaggggcaaa aacgaaagcg
1501 caagaaatcc cggtataagt cctggagcgt gtacgttggt gcccgctgct gtctaatgcc
1561 ctggagcctc cctggccccc atccctgtgg gccttgctca gagcggagaa agcatttgtt
1621 tgtacaagat ccgcagacgt gtaaatgttc ctgcaaaaac acagactcgc gttgcaaggc
1681 gaggcagctt gagttaaacg aacgtacttg cagatgtgac aagccgaggc ggtgagccgg
1741 gcaggaggaa ggagcctccc tcagggtttc gggaaccaga tctctcacca ggaaagactg
1801 atacagaacg atcgatacag aaaccacgct gccgccacca caccatcacc atcgacagaa
1861 cagtccttaa tccagaaacc tgaaatgaag gaagaggaga ctctgcgcag agcactttgg
1921 gtccggaggg cgagactccg gcggaagcat tcccgggcgg gtgacccagc acggtccctc
1981 ttggaattgg attcgccatt ttatttttct tgctgctaaa tcaccgagcc cggaagatta
2041 gagagtttta tttctgggat tcctgtagac acacccaccc acatacatac atttatatat
2101 atatatatta tatatatata aaaataaata tctctatttt atatatataa aatatatata
2161 ttcttttttt aaattaacag tgctaatgtt attggtgtct tcactggatg tatttgactg
2221 ctgtggactt gagttgggag gggaatgttc ccactcagat cctgacaggg aagaggagga
2281 gatgagagac tctggcatga tctttttttt gtcccacttg gtggggccag ggtcctctcc
2341 cctgcccagg aatgtgcaag gccagggcat gggggcaaat atgacccagt tttgggaaca
2401 ccgacaaacc cagccctggc gctgagcctc tctaccccag gtcagacgga cagaaagaca
2461 gatcacaggt acagggatga ggacaccggc tctgaccagg agtttgggga gcttcaggac
2521 attgctgtgc tttggggatt ccctccacat gctgcacgcg catctcgccc ccaggggcac
2581 tgcctggaag attcaggagc ctgggcggcc ttcgcttact ctcacctgct tctgagttgc
2641 ccaggagacc actggcagat gtcccggcga agagaagaga cacattgttg gaagaagcag
2701 cccatgacag ctccccttcc tgggactcgc cctcatcctc ttcctgctcc ccttcctggg
2761 gtgcagccta aaaggaccta tgtcctcaca ccattgaaac cactagttct gtccccccag
2821 gagacctggt tgtgtgtgtg tgagtggttg accttcctcc atcccctggt ccttcccttc
2881 ccttcccgag gcacagagag acagggcagg atccacgtgc ccattgtgga ggcagagaaa
2941 agagaaagtg ttttatatac ggtacttatt taatatccct ttttaattag aaattaaaac
3001 agttaattta attaaagagt agggtttttt ttcagtattc ttggttaata tttaatttca
3061 actatttatg agatgtatct tttgctctct cttgctctct tatttgtacc ggtttttgta
3121 tataaaattc atgtttccaa tctctctctc cctgatcggt gacagtcact agcttatctt
3181 gaacagatat ttaattttgc taacactcag ctctgccctc cccgatcccc tggctcccca
3241 gcacacattc ctttgaaata aggtttcaat atacatctac atactatata tatatttggc
3301 aacttgtatt tgtgtgtata tatatatata tatgtttatg tatatatgtg attctgataa
3361 aatagacatt gctattctgt tttttatatg taaaaacaaa acaagaaaaa atagagaatt
3421 ctacatacta aatctctctc cttttttaat tttaatattt gttatcattt atttattggt
3481 gctactgttt atccgtaata attgtgggga aaagatatta acatcacgtc tttgtctcta
3541 gtgcagtttt tcgagatatt ccgtagtaca tatttatttt taaacaacga caaagaaata
3601 cagatatatc ttaaaaaaaa aaaagcattt tgtattaaag aatttaattc tgatctcaaa
Homo sapiens interleukin 1 receptor antagonist (IL1RN), transcript variant 2, mRNA
(NCBI Reference Sequence: NM_173841.3)
(SEQ ID NO: 19)
      1 agagtctgga agacctcaga agacctcctg tcctatgagg ccctccccat ggctttagct
    61 gacttgtatg aagaaggagg tggaggagga ggagaaggtg aagacaatgc tgactcaaag
  121 gagacgatct gccgaccctc tgggagaaaa tccagcaaga tgcaagcctt cagaatctgg
  181 gatgttaacc agaagacctt ctatctgagg aacaaccaac tagttgctgg atacttgcaa
  241 ggaccaaatg tcaatttaga agaaaagata gatgtggtac ccattgagcc tcatgctctg
  301 ttcttgggaa tccatggagg gaagatgtgc ctgtcctgtg tcaagtctgg tgatgagacc
  361 agactccagc tggaggcagt taacatcact gacctgagcg agaacagaaa gcaggacaag
  421 cgcttcgcct tcatccgctc agacagtggc cccaccacca gttttgagtc tgccgcctgc
  481 cccggttggt tcctctgcac agcgatggaa gctgaccagc ccgtcagcct caccaatatg
  541 cctgacgaag gcgtcatggt caccaaattc tacttccagg aggacgagta gtactgccca
  601 ggcctgcctg ttcccattct tgcatggcaa ggactgcagg gactgccagt ccccctgccc
  661 cagggctccc ggctatgggg gcactgagga ccagccattg aggggtggac cctcagaagg
  721 cgtcacaaca acctggtcac aggactctgc ctcctcttca actgaccagc ctccatgctg
  781 cctccagaat ggtctttcta atgtgtgaat cagagcacag cagcccctgc acaaagccct
  841 tccatgtcgc ctctgcattc aggatcaaac cccgaccacc tgcccaacct gctctcctct
  901 tgccactgcc tcttcctccc tcattccacc ttcccatgcc ctggatccat caggccactt
  961 gatgaccccc aaccaagtgg ctcccacacc ctgttttaca aaaaagaaaa gaccagtcca
1021 tgagggaggt ttttaagggt ttgtggaaaa tgaaaattag gatttcatga tttttttttt
1081 tcagtccccg tgaaggagag cccttcattt ggagattatg ttctttcggg gagaggctga
1141 ggacttaaaa tattcctgca tttgtgaaat gatggtgaaa gtaagtggta gcttttccct
1201 tctttttctt ctttttttgt gatgtcccaa cttgtaaaaa ttaaaagtta tggtactatg
1261 ttagccccat aatttttttt ttccttttaa aacacttcca taatctggac tcctctgtcc
1321 aggcactgct gcccagcctc caagctccat ctccactcca gattttttac agctgcctgc
1381 agtactttac ctcctatcag aagtttctca gctcccaagg ctctgagcaa atgtggctcc
1441 tctttttctt ctttttttgt gatgtcccaa cttgtaaaaa ttaaaagtta tagagcttct
1501 ttagccccat aatttttttt ttccttttaa aacacttcca taatctggac caccgggctg
1561 ggagctctgc agagcaggaa acatgactcg tatatgtctc aggtccctgc agggccaagc
1621 acctagcctc gctcttggca ggtactcagc gaatgaatgc tgtatatgtt gggtgcaaag
1681 ttccctactt cctgtgactt cagctctgtt ttacaataaa atcttgaaaa tgcc
Homo sapiens C-C motif chemokine ligand 18 (CCL18), mRNA (NCBI Reference
Sequence: NM_002988.4)
(SEQ ID NO: 20)
      1 agaaggaggc caggagttgt gagtttccaa gccccagctc actctgacca cttctctgcc
    61 tgcccagcat catgaagggc cttgcagctg ccctccttgt cctcgtctgc accatggccc
  121 tctgctcctg tgcacaagtt ggtaccaaca aagagctctg ctgcctcgtc tatacctcct
  181 ggcagattcc acaaaagttc atagttgact attctgaaac cagcccccag tgccccaagc
  241 caggtgtcat cctcctaacc aagagaggcc ggcagatctg tgctgacccc aataagaagt
  301 gggtccagaa atacatcagc gacctgaagc tgaatgcctg aggggcctgg aagctgcgag
  361 ggcccagtga acttggtggg cccaggaggg aacaggagcc tgagccaggg caatggccct
  421 gccaccctgg aggctacctc ttctaagagt cccatctgct atgcccagcc acattaacta
  481 actttaatct tagtttatgc atcatatttc attttgaaat tgatttctat tgttgagctg
  541 cattatgaaa ttagtatttt ctctgacatc tcatgacatt gtctttatca tcctttcccc
  601 tttcccttca actcttcgta cattcaatgc atggatcaat cagtgtgatt agctttctca
  661 gcagacattg tgccatatgt atcaaatgac aaatctttat tgaatggttt tgctcagcac
  721 caccttttaa tatattggca gtacttatta tataaaaggt aaaccagcat tctcactgtg
  781 acgactctgt tgattttgtt tcactaatcg gaatcacaga ctgagaggaa ttctggggga
  841 ggagtaggga aatatgaaaa ggggcagaaa cagcaagata gggaccaagt acttctacag
  901 tcataccaga catttccctg gagatacttt cctgaaaagt tgaaacagac accattagtt
  961 tataaaccat attgtaactg aaatgtgata gaaaaatttt ctacttaaat gaatatcaag
1021 atgacgctgc aatgcatatt tacgcacaca agctcatttt cataaatgaa gctttcctca
1081 gaatgatggc taacatctgt tgaggtctta ccaagtgctg ggttttgggc taagtactta
1141 tattagtaaa agtctagcaa atactgccca tagtctagca aggactcctt acctggaagt
1201 tgctgaaagc cttggtaatg ttatctttgt tctccactct gcttttgggg aggatgtttt
1261 ccatgactat acgagatgag gcttggggct gggtggccag agttagcaaa taaaaataca
1321 gaatggatac ta
```

In another aspect, the present disclosure provides a method of diagnosing atopic dermatitis (AD), comprising detecting in a sample obtained from a subject suspected of having AD the expression level of at least one, at least two, at least three, at least four, at least five, or all six biomarker(s) selected from CCL20, CCL22, CCL27, VEGF, IL1RA, and CCL18, and comparing the expression level of the biomarkers to a reference level, wherein the reference level is the corresponding level of expression for each biomarker in a skin sample from an individual that does not have AD or a sample of non-lesional skin from the subject suspected of having AD. Additionally or alternatively, the present disclosure provides a diagnostic agent that detects in a subject suspected of having AD the expression level of at least one, at least two, at least three, at least four, at least five, or all six biomarker(s) selected from CCL20, CCL22, CCL27, VEGF, IL1RA, and CCL18, wherein the expression level of the biomarkers can be compared to a reference level corresponding to the level of expression for each biomarker in a skin sample from an individual that does not have AD or a sample of non-lesional skin from the subject suspected of having AD. In some embodiments, the sample obtained from the subject suspected of having AD is a skin sample, which optionally comprises a lesion. For the purposes of the foregoing aspects, a skin sample may comprise, consist of, or consist essentially of stratum corneum.

In another aspect, the present disclosure provides a method of detecting inflammatory biomarkers in a sample, comprising detecting in a sample obtained from a subject suspected of having AD the expression level of at least one, at least two, at least three, at least four, at least five, or all six biomarker(s) selected from CCL20, CCL22, CCL27, VEGF, IL1RA, and CCL18. The method may further comprise comparing the expression level of the biomarker(s) to a reference level, wherein the reference level is the corresponding level of expression for each biomarker in a skin sample from an individual that does not have AD or a sample of non-lesional skin from the subject suspected of having AD. Additionally or alternatively, the present disclosure provides a diagnostic agent that detects inflammatory biomarkers in a sample obtained from a subject suspected of having, wherein the inflammatory biomarkers comprise at least one, at least two, at least three, at least four, at least five, or all six biomarker(s) selected from CCL20, CCL22, CCL27, VEGF, IL1RA, and CCL18, wherein the expression level of the biomarkers can be compared to a reference level corresponding to the level of expression for each biomarker in a skin sample from an individual that does not have AD. In some embodiments, the sample obtained from the subject suspected of having AD is a skin sample, which optionally comprises a lesion. For the purposes of the foregoing aspects, a skin sample may comprise, consist of, or consist essentially of stratum corneum.

For the purposes of the foregoing aspects, expression of CCL18 in the lesional skin of a subject with AD (or suspected of having AD) may be about 300 ppm (when calculated as a ratio of biomarker protein to total protein in a sample), about 310 ppm, about 320 ppm, about 330 ppm, about 340 ppm, about 350 ppm, about 360 ppm, about 370 ppm, about 380 ppm, about 390 ppm, about 400 ppm, or more. Additionally or alternatively, expression of CCL18 in the lesional skin of a subject with AD (or suspected of having AD) may be at least 300 ppm (when calculated as a ratio of biomarker protein to total protein in a sample), at least 310 ppm, at least 320 ppm, at least 330 ppm, at least 340 ppm, at least 350 ppm, at least 360 ppm, at least 370 ppm, at least 380 ppm, at least 390 ppm, at least 400 ppm, or more. Additionally or alternatively, expression of CCL18 in the lesional skin of a subject with AD (or suspected of having AD) may be about 1.5-fold, about 2-fold, about 2.1-fold, about 2.2-fold, about 2.3-fold, about 2.4-fold, about 2.5-fold, about 2.6-fold, about 2.7-fold, about 2.8-fold, about 2.9-fold, or about 3-fold or more than the expression level of CCL18 in a reference sample (e.g., non-lesional skin of the subject or skin from a subject without AD). Additionally or alternatively, expression of CCL18 in the lesional skin of a subject with AD (or suspected of having AD) may be at least 1.5-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, or at least 3-fold or more than the expression level of CCL18 in a reference sample (e.g., non-lesional skin of the subject or skin from a subject without AD). Additionally or alternatively, expression of CCL18 in the lesional skin of a subject with AD (or suspected of having AD) may be about 150%, about 200%, about 210%, about 220%, about 230%, about 240%, about 250%, about 260%, about 270%, about 280%, about 290%, or about 300% or more of the expression level of CCL18 in a reference sample (e.g., non-lesional skin of the subject or skin from a subject without AD). Additionally or alternatively, expression of CCL18 in the lesional skin of a subject with AD (or suspected of having AD) may be at least 150%, at least 200%, at least 210%, at least 220%, at least 230%, at least 240%, at least 250%, at least 260%, at least 270%, at least 280%, at least 290%, or at least 300% or more of the expression level of CCL18 in a reference sample (e.g., non-lesional skin of the subject or skin from a subject without AD).

For the purposes of the foregoing aspects, expression of CCL20 in the lesional skin of a subject with AD (or suspected of having AD) may be about 15.5 ppm (when calculated as a ratio of biomarker protein to total protein in a sample), about 15.6 ppm, about 15.7 ppm, about 15.8 ppm, about 15.9 ppm, about 16 ppm, about 16.1 ppm, about 16.2 ppm, about 16.3 ppm, about 16.4 ppm, about 16.5 ppm, about 16.6 ppm, about 16.7 ppm, about 16.8 ppm, about 16.9 ppm, about 17 ppm or more. Additionally or alternatively, expression of CCL20 in the lesional skin of a subject with AD (or suspected of having AD) may be at least 15.5 ppm (when calculated as a ratio of biomarker protein to total protein in a sample), at least 15.6 ppm, at least 15.7 ppm, at least 15.8 ppm, at least 15.9 ppm, at least 16 ppm, at least 16.1 ppm, at least 16.2 ppm, at least 16.3 ppm, at least 16.4 ppm, at least 16.5 ppm, at least 16.6 ppm, at least 16.7 ppm, at least 16.8 ppm, at least 16.9 ppm, at least 17 ppm or more. Additionally or alternatively, expression of CCL20 in the lesional skin of a subject with AD (or suspected of having AD) may be about 1.1-fold, about 1.2-fold, about 1.3-fold, about 1.4-fold, about 1.5-fold, about 1.6-fold, about 1.7-fold, about 1.8-fold, about 1.9-fold, about 2-fold or more than the expression level of CCL20 in a reference sample (e.g., non-lesional skin of the subject or skin from a subject without AD). Additionally or alternatively, expression of CCL20 in the lesional skin of a subject with AD (or suspected of having AD) may be at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold or more than the expression level of CCL20 in a reference sample (e.g., non-lesional skin of the subject or skin from a subject without AD). Additionally or alternatively, expression of CCL20 in the lesional skin of a subject with AD (or suspected of having AD) may be about 110%, about 120%, about 125%, about 130%, about 135%, about 140%, about 145%, about 150%, about 155%, about 160%, about 165%, about 170%, or about 175% or more of the expression level of CCL20 in a reference sample (e.g., non-lesional skin of the subject or skin from a subject without AD). Additionally or alternatively, expression of CCL20 in the lesional skin of a subject with AD (or suspected of having AD) may be at least 110%, at least 120%, at least 125%, at least 130%, at least 135%, at least 140%, at least 145%, at least 150%, at least 155%, at least 160%, at least 165%, at least 170%, or at least 175% or more of the expression level of CCL20 in a reference sample (e.g., non-lesional skin of the subject or skin from a subject without AD).

For the purposes of the foregoing aspects, expression of CCL22 in the lesional skin of a subject with AD (or suspected of having AD) may be about 1550 ppm (when calculated as a ratio of biomarker protein to total protein in a sample), about 1560 ppm, about 1570 ppm, about 1580 ppm, about 1590 ppm, about 1600 ppm, about 1610 ppm, about 1620 ppm, about 1630 ppm, about 1640 ppm, about 1650 ppm, about 1660 ppm, about 1670 ppm, about 1680 ppm, about 1690 ppm, about 1700 ppm or more. Additionally or alternatively, expression of CCL22 in the lesional skin of a subject with AD (or suspected of having AD) may be at least 1550 ppm (when calculated as a ratio of biomarker protein to total protein in a sample), at least 1560 ppm, at least 1570 ppm, at least 1580 ppm, at least 1590 ppm, at least 1600 ppm, at least 1610 ppm, at least 1620 ppm, at least 1630 ppm, at least 1640 ppm, at least 1650 ppm, at least 1660 ppm, at least 1670 ppm, at least 1680 ppm, at least 1690 ppm, at least 1700 ppm or more. Additionally or alternatively, expression of CCL22 in the lesional skin of a subject with AD (or suspected of having AD) may be about 1.01-fold, about 1.02-fold, about 1.03-fold, about 1.04-fold, about 1.05-fold, about 1.06-fold, about 1.07-fold, about 1.08-fold, about 1.09-fold, about 1.1-fold, about 1.2-fold, about 1.3-fold, about 1.4-fold, about 1.5-fold, about 1.6-fold, about 1.7-fold, about 1.8-fold, about 1.9-fold, about 2-fold or more than the expression level of CCL22 in a reference sample (e.g., non-lesional skin of the subject or skin from a subject without AD) or the expression level may be about the same between the lesional skin and the reference sample. Additionally or alternatively, expression of CCL22 in the lesional skin of a subject with AD (or suspected of having AD) may be at least 1.01-fold, at least 1.02-fold, at least 1.03-fold, at least 1.04-fold, at least 1.05-fold, at least 1.06-fold, at least 1.07-fold, at least 1.08-fold, at least 1.09-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold or more than the expression level of CCL22 in a reference sample (e.g., non-lesional skin of the subject or skin from a subject without AD) or the expression level may be about the same between the lesional skin and the reference sample. Additionally or alternatively, expression of CCL22 in the lesional skin of a subject with AD (or suspected of having AD) may be about 101%, about 102%, about 103%, about 104%, about 105%, about 106%, about 107%, about 108%, about 109%, about 110%, about 120%, about 125%, about 130%, about 135%, about 140%, about 145%, about 150%, about 155%, about 160%, about 165%, about 170%, or about 175% or more of the expression level of CCL22 in a reference sample (e.g., non-lesional skin of the subject or skin from a subject without AD). Additionally or alternatively, expression of CCL22 in the lesional skin of a subject with AD (or suspected of having AD) may be at least 101%, at least 102%, at least 103%, at least 104%, at least 105%, at least 106%, at least 107%, at least 108%, at least 109%, at least 110%, at least 120%, at least 125%, at least 130%, at least 135%, at least 140%, at least 145%, at least 150%, at least 155%, at least 160%, at least 165%, at least 170%, or at least 175% or more of the expression level of CCL22 in a reference sample (e.g., non-lesional skin of the subject or skin from a subject without AD).

For the purposes of the foregoing aspects, expression of CCL27 in the lesional skin of a subject with AD (or suspected of having AD) may be about 50 ppm (when calculated as a ratio of biomarker protein to total protein in a sample), about 51 ppm, about 52 ppm, about 53 ppm, about 54 ppm, about 55 ppm, about 56 ppm, about 57 ppm, about 58 ppm, about 59 ppm, about 60 ppm, about 61 ppm, about 62 ppm, about 63 ppm, about 64 ppm, about 65 ppm, about 66 ppm, about 67 ppm, about 68 ppm, about 69 ppm, about 70 ppm, about 71 ppm, about 72 ppm, about 73 ppm, about 74 ppm, about 75 ppm or more. Additionally or alternatively, expression of CCL27 in the lesional skin of a subject with AD (or suspected of having AD) may be at least 50 ppm (when calculated as a ratio of biomarker protein to total protein in a sample), at least 51 ppm, at least 52 ppm, at least 53 ppm, at least 54 ppm, at least 55 ppm, at least 56 ppm, at least 57 ppm, at least 58 ppm, at least 59 ppm, at least 60 ppm, at least 61 ppm, at least 62 ppm, at least 63 ppm, at least 64 ppm, at least 65 ppm, at least 66 ppm, at least 67 ppm, at least 68 ppm, at least 69 ppm, at least 70 ppm, at least 71 ppm, at least 72 ppm, at least 73 ppm, at least 74 ppm, at least 75 ppm or more. Additionally or alternatively, expression of CCL27 in the lesional skin of a subject with AD (or suspected of having AD) may be about 1.5-fold, about 2-fold, about 2.1-fold, about 2.2-fold, about 2.3-fold, about 2.4-fold, about 2.5-fold, about 2.6-fold, about 2.7-fold, about 2.8-fold, about 2.9-fold, or about 3-fold or more than the expression level of CCL27 in a reference sample (e.g., non-lesional skin of the subject or skin from a subject without AD). Additionally or alternatively, expression of CCL27 in the lesional skin of a subject with AD (or suspected of having AD) may be at least 1.5-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, or at least 3-fold or more than the expression level of CCL27 in a reference sample (e.g., non-lesional skin of the subject or skin from a subject without AD). Additionally or alternatively, expression of CCL27 in the lesional skin of a subject with AD (or suspected of having AD) may be about 150%, about 175%, about 200%, about 210%, about 220%, about 225% about 230%, about 240%, about 250%, about 260%, about 270%, about 280%, about 290%, or about 300% or more of the expression level of CCL27 in a reference sample (e.g., non-lesional skin of the subject or skin from a subject without AD). Additionally or alternatively, expression of CCL27 in the lesional skin of a subject with AD (or suspected of having AD) may be at least 150%, at least 175%, at least 200%, at least 210%, at least 220%, at least 225%, at least 230%, at least 240%, at least 250%, at least 260%, at least 270%, at least 280%, at least 290%, or at least 300% or more of the expression level of CCL27 in a reference sample (e.g., non-lesional skin of the subject or skin from a subject without AD).

For the purposes of the foregoing aspects, expression of VEGF in the lesional skin of a subject with AD (or suspected of having AD) may be about 150 ppm (when calculated as a ratio of biomarker protein to total protein in a sample), about 151 ppm, about 152 ppm, about 153 ppm, about 154 ppm, about 155 ppm, about 156 ppm, about 157 ppm, about 158 ppm, about 159 ppm, about 160 ppm, about 161 ppm, about 162 ppm, about 163 ppm, about 164 ppm, about 165 ppm, about 166 ppm, about 167 ppm, about 168 ppm, about 169 ppm, about 170 ppm, about 171 ppm, about 172 ppm, about 173 ppm, about 174 ppm, about 175 ppm or more. Additionally or alternatively, expression of CCL27 in the lesional skin of a subject with AD (or suspected of having AD) may be at least 150 ppm (when calculated as a ratio of biomarker protein to total protein in a sample), at least 151 ppm, at least 152 ppm, at least 153 ppm, at least 154 ppm, at least 155 ppm, at least 156 ppm, at least 157 ppm, at least 158 ppm, at least 159 ppm, at least 160 ppm, at least 161 ppm, at least 162 ppm, at least 163 ppm, at least 164 ppm, at least 165 ppm, at least 166 ppm, at least 167 ppm, at least 168 ppm, at least 169 ppm, at least 170 ppm, at least 171 ppm, at least 172 ppm, at least 173 ppm, at least 174 ppm, at least 75 ppm or more. Additionally or alternatively, expression of VEGF in the lesional skin of a subject with AD (or suspected of having AD) may be about 1.01-fold, about 1.02-fold, about 1.03-fold, about 1.04-fold, about 1.05-fold, about 1.06-fold, about 1.07-fold, about 1.08-fold, about 1.09-fold, about 1.1-fold, about 1.2-fold, about 1.3-fold, about 1.4-fold, about 1.5-fold, about 1.6-fold, about 1.7-fold, about 1.8-fold, about 1.9-fold, about 2-fold or more than the expression level of VEGF in a reference sample (e.g., non-lesional skin of the subject or skin from a subject without AD) or the expression level may be about the same between the lesional skin and the reference sample. Additionally or alternatively, expression of VEGF in the lesional skin of a subject with AD (or suspected of having AD) may be at least 1.01-fold, at least 1.02-fold, at least 1.03-fold, at least 1.04-fold, at least 1.05-fold, at least 1.06-fold, at least 1.07-fold, at least 1.08-fold, at least 1.09-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold or more than the expression level of VEGF in a reference sample (e.g., non-lesional skin of the subject or skin from a subject without AD) or the expression level may be about the same between the lesional skin and the reference sample. Additionally or alternatively, expression of VEGF in the lesional skin of a subject with AD (or suspected of having AD) may be about 101%, about 102%, about 103%, about 104%, about 105%, about 106%, about 107%, about 108%, about 109%, about 110%, about 120%, about 125%, about 130%, about 135%, about 140%, about 145%, about 150%, about 155%, about 160%, about 165%, about 170%, or about 175% or more of the expression level of VEGF in a reference sample (e.g., non-lesional skin of the subject or skin from a subject without AD). Additionally or alternatively, expression of VEGF in the lesional skin of a subject with AD (or suspected of having AD) may be at least 101%, at least 102%, at least 103%, at least 104%, at least 105%, at least 106%, at least 107%, at least 108%, at least 109%, at least 110%, at least 120%, at least 125%, at least 130%, at least 135%, at least 140%, at least 145%, at least 150%, at least 155%, at least 160%, at least 165%, at least 170%, or at least 175% or more of the expression level of VEGF in a reference sample (e.g., non-lesional skin of the subject or skin from a subject without AD).

For the purposes of the foregoing aspects, expression of IL1RA in the lesional skin of a subject with AD (or suspected of having AD) may be about 207000 ppm (when calculated as a ratio of biomarker protein to total protein in a sample), about 207500 ppm, about 208000 ppm, about 208500 ppm, about 209000 ppm, about 209500 ppm, about 210000 ppm, about 210500 ppm, about 211000 ppm, about 211500 ppm, about 212000 ppm, about 212500 ppm, about 213000 ppm, about 213500 ppm, about 214000 ppm, about 214500 ppm, about 215000 ppm, about 215500 ppm, about 216000 ppm, about 216500 ppm, about 217000 ppm, about 217500 ppm, about 218000 ppm, about 218500 ppm, about 219000 ppm, about 219500, about 220000, about 221000, about 222000, about 223000, about 224000, about 225000, about 226000, about 227000, about 228000, about 229000, about 230000, about 231000, about 232000, about 233000, about 234000, about 235000 ppm or more. Additionally or alternatively, expression of CCL27 in the lesional skin of a subject with AD (or suspected of having AD) may be at least 207000 ppm (when calculated as a ratio of biomarker protein to total protein in a sample), at least 207500 ppm, at least 208000 ppm, at least 208500 at least, at least 209000 ppm, at least 209500 ppm, at least 210000 ppm, at least 210500 ppm, at least 211000 ppm, at least 211500 ppm, at least 212000 ppm, at least 212500 ppm, at least 213000 ppm, at least 213500 ppm, at least 214000 ppm, at least 214500 ppm, at least 215000 ppm, at least 215500 ppm, at least 216000 ppm, at least 216500 ppm, at least 217000 ppm, at least 217500 ppm, at least 218000 ppm, at least 218500 ppm, at least 219000 ppm, at least 219500, at least 220000, at least 221000, at least 222000, at least 223000, at least 224000, at least 225000, at least 226000, at least 227000, at least 228000, at least 229000, at least 230000, at least 231000, at least 232000, at least 233000, at least 234000, at least 235000 ppm or more. Additionally or alternatively, expression of IL1RA in the lesional skin of a subject with AD (or suspected of having AD) may be about 1.01-fold, about 1.02-fold, about 1.03-fold, about 1.04-fold, about 1.05-fold, about 1.06-fold, about 1.07-fold, about 1.08-fold, about 1.09-fold, about 1.1-fold, about 1.2-fold, about 1.3-fold, about 1.4-fold, about 1.5-fold, about 1.6-fold, about 1.7-fold, about 1.8-fold, about 1.9-fold, about 2-fold or more than the expression level of IL1RA in a reference sample (e.g., non-lesional skin of the subject or skin from a subject without AD) or the expression level may be about the same between the lesional skin and the reference sample. Additionally or alternatively, expression of IL1RA in the lesional skin of a subject with AD (or suspected of having AD) may be at least 1.01-fold, at least 1.02-fold, at least 1.03-fold, at least 1.04-fold, at least 1.05-fold, at least 1.06-fold, at least 1.07-fold, at least 1.08-fold, at least 1.09-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold or more than the expression level of IL1RA in a reference sample (e.g., non-lesional skin of the subject or skin from a subject without AD) or the expression level may be about the same between the lesional skin and the reference sample. Additionally or alternatively, expression of IL1RA in the lesional skin of a subject with AD (or suspected of having AD) may be about 101%, about 102%, about 103%, about 104%, about 105%, about 106%, about 107%, about 108%, about 109%, about 110%, about 115%, about 120%, about 125%, about 130%, about 135%, about 140%, about 145%, about 150%, about 155%, about 160%, about 165%, about 170%, or about 175% or more of the expression level of IL1RA in a reference sample (e.g., non-lesional skin of the subject or skin from a subject without AD). Additionally or alternatively, expression of IL1RA in the lesional skin of a subject with AD (or suspected of having AD) may be at least 101%, at least 102%, at least 103%, at least 104%, at least 105%, at least 106%, at least 107%, at least 108%, at least 109%, at least 110%, at least 115%, at least 120%, at least 125%, at least 130%, at least 135%, at least 140%, at least 145%, at least 150%, at least 155%, at least 160%, at least 165%, at least 170%, or at least 175% or more of the expression level of IL1RA in a reference sample (e.g., non-lesional skin of the subject or skin from a subject without AD).

### IV. Therapeutic Antibodies and Interleukin 31 Receptor Subunit Alpha (IL-31RA)

Interleukin-31 (IL-31) is a neuro-inflammatory cytokine that could activate both structural, immune cells and peripheral nerves. It has been involved in a number of chronic inflammatory diseases, including atopic dermatitis. IL-31 is produced by a variety of cells, including type 2 helper (Th2) T-cells. IL-31 sends signals through a receptor complex made of Interleukin 31 receptor subunit alpha (“IL-31RA,” also known as NR10, glm-r, and GPL) and oncostatin M receptor R (OSMR3) expressed in immune and epithelial cells, as well as in a subset of neurons.

IL-31RA forms a heterodimer with oncostatin M receptor (OSMR) when functioning as an IL-31 receptor. There are multiple known splicing variants of human-derived IL-31RA (WO 00/075314): NR10.1 consists of 662 amino acids and contains a transmembrane domain. NR10.2 is a soluble receptor-like protein consisting of 252 amino acids without the transmembrane domain. Further, known IL-31RA splicing variants that function as transmembrane receptor proteins include NR10.3 and IL-31RAv3. Preferred IL-31RA variants include NR10.3 (also referred to as ILRAv4 (Nat Immunol 5, 752-60, 2004) and IL-31RAv3. NR 10.3 (IL31RAv4) consists of 662 amino acids (WO 00/075314; Nat Immunol 5, 752-60, 2004) and IL31RAv3 consists of 732 amino acids (GenBank Accession No: NM-139017).

The amino acid sequence of IL31RAv4 is:

(SEQ ID NO: 1)
MKLSPQPSCVNLGMMWTWALWMLPSLCKFSLAALPAKPENISCVYYYRK
NLTCTWSPGKETSYTQYTVKRTYAFGEKHDNCTTNSSTSENRASCSFFL
PRITIPDNYTIEVEAENGDGVIKSHMTYWRLENIAKTEPPKIFRVKPVL
GIKRMIQIEWIKPELAPVSSDLKYTLRFRTVNSTSWMEVNFAKNRKDKN
QTYNLTGLQPFTEYVIALRCAVKESKFWSDWSQEKMGMTEEEAPCGLEL
WRVLKPAEADGRRPVRLLWKKARGAPVLEKTLGYNIWYYPESNTNLTET
MNTTNQQLELHLGGESFWVSMISYNSLGKSPVATLRIPAIQEKSFQCIE
VMQACVAEDQLVVKWQSSALDVNTWMIEWFPDVDSEPTTLSWESVSQAT
NWTIQQDKLKPFWCYNISVYPMLHDKVGEPYSIQAYAKEGVPSEGPETK
VENIGVKTVTITWKEIPKSERKGIICNYTIFYQAEGGKGFSKTVNSSIL
QYGLESLKRKTSYIVQVMASTSAGGTNGTSINFKTLSFSVFEIILITSL
IGGGLLILIILTVAYGLKKPNKLTHLCWPTVPNPAESSIATWHGDDFKD
KLNLKESDDSVNTEDRILKPCSTPSDKLVIDKLVVNFGNVLQEIFTDEA
RTGQENNLGGEKNGTRILSSCPTSI

The amino acid sequence of IL31RAv3 is:

(SEQ ID NO: 2)
MMWTWALWMLPSLCKFSLAALPAKPENISCVYYYRKNLTCTWSPGKETS
YTQYTVKRTYAFGEKHDNCTTNSSTSENRASCSFFLPRITIPDNYTIEV
EAENGDGVIKSHMTYWRLENIAKTEPPKIFRVKPVLGIKRMIQIEWIKP
ELAPVSSDLKYTLRFRTVNSTSWMEVNFAKNRKDKNQTYNLTGLQPFTE
YVIALRCAVKESKFWSDWSQEKMGMTEEEAPCGLELWRVLKPAEADGRR
PVRLLWKKARGAPVLEKTLGYNIWYYPESNTNLTETMNTTNQQLELHLG
GESFWVSMISYNSLGKSPVATLRIPAIQEKSFQCIEVMQACVAEDQLVV
KWQSSALDVNTWMIEWFPDVDSEPTTLSWESVSQATNWTIQQDKLKPFW
CYNISVYPMLHDKVGEPYSIQAYAKEGVPSEGPETKVENIGVKTVTITW
KEIPKSERKGIICNYTIFYQAEGGKGFSKTVNSSILQYGLESLKRKTSY
IVQVMASTSAGGTNGTSINFKTLSFSVFEIILITSLIGGGLLILIILTV
AYGLKKPNKLTHLCWPTVPNPAESSIATWHGDDFKDKLNLKESDDSVNT
EDRILKPCSTPSDKLVIDKLVVNFGNVLQEIFTDEARTGQENNLGGEKN
GYVTCPFRPDCPLGKSFEELPVSPEIPPRKSQYLRSRMPEGTRPEAKEQ
LLFSGQSLVPDHLCEEGAPNPYLKNSVTAREFLVSEKLPEHTKGEV

Mouse-derived IL-31RA comprises the amino acid sequence:

(SEQ ID NO: 3)
MWTLALWAFSFLCKFSLAVLPTKPENISCVFYFDRNLTCTWRPEKETND
TSYIVTLTYSYGKSNYSDNATEASYSFPRSCAMPPDICSVEVQAQNGDG
KVKSDITYWHLISIAKTEPPIILSVNPICNRMFQIQWKPREKTRGFPLV
CMLRFRTVNSSRWTEVNFENCKQVCNLTGLQAFTEYVLALRFRFNDSRY
WSKWSKEETRVTMEEVPHVLDLWRILEPADMNGDRKVRLLWKKARGAPV
LEKTFGYHIQYFAENSTNLTEINNITTQQYELLLMSQAHSVSVTSFNSL
GKSQEAILRIPDVHEKTFQYIKSMKAYIAEPLLVVNWQSSIPAVDTWIV
EWLPEAAMSKFPALSWESVSQVTNWTIEQDKLKPFTCYNISVYPVLGHR
VGEPYSIQAYAKEGTPLKGPETRVENIGLRTATITWKEIPKSARNGFIN
NYTVFYQAEGGKELSKTVNSHALQCDLESLTRRTSYTVWVMASTRAGGT
NGVRINFKTLSISVFEIVLLTSLVGGGLLLLSIKTVTFGLRKPNRLTPL
CCPDVPNPAESSLATWLGDGFKKSNMKETGNSGDTEDVVLKPCPVPADL
IDKLVVNFENFLEVVLTEEAGKGQASILGGEANEYVTSPSRPDGPPGKS
FKEPSVLTEVASEDSHSTCSRMADEAYSELARQPSSSCQSPGLSPPRED
QAQNPYLKNSVTTREFLVHENIPEHSKGEV

Cynomolgus monkey-derived IL-31RA comprises the amino acid sequence:

(SEQ ID NO: 4)
MMWTWALWMFPLLCKFGLAALPAKPENISCVYYYRKNLTCTWSPGKETS
YTQYTAKRTYAFGKKHDNCTTSSSTSENRASCSFFLPRITIPDNYTIEV
EAENGDGVIKSDMTCWRLEDIAKTEPPEIFSVKPVLGIKRMIRIEWIKP
ELAPVSSDLKYALRFRTVNSTSWMEVNFAKNRKDTNQTYNLMGLQAFTE
YVVALRCAVKESKFWSDWSQEKMGMTEEEAPCGLELWRVLKPTEVDGRR
PVRLLWKKARGAPVLEKTLGYNIWYFPENNTNLTETVNTTNQQLELHLG
GESYWVSMISYNSLGKSPVTTLRIPAIQEKSFRCIEVMQACLAEDQLVV
KWQSSALDVNTWMIEWFPDMDSEHPTLSWESVSQATNWTIQQDKLKPFW
CYNISVYPMLHDKVGEPYSIQAYAKEGIPSKGPETKVENIGVKTVTITW
KEIPKSERKGIICNYTIFYQAEGGKGFSKTVNSSILQYGLESLKRKTSY
TVRVMASTSAGGINGTSINFKTLSFSVFEIILITSLIGGGLLILIILTV
AYGLKKPNKLTHLCWPSVPNPAESSIATWRGDDFKDKLNLKESDDSVNT
EDRILKPCSTPSDKLVIDKSVVNFGNVLQEMFTDEARTGQENNLGGEKN
EYVTHPFRADCPLGKSFEELPVSPEIPPRKSQYLRSRMPEGTCLEAEEQ
LLVSGQSLESLAPDHVREAAAPNPYLKNSVTTREFLVSQKLPEHTKGEV

For the purposes of the present disclosure, an anti-IL-31RA antibody (i.e., a therapeutic antibody), such as nemolizumab, must bind to at least human IL-31RA or a splice variant thereof.

As used herein, the term “antibody” collectively refers to immunoglobulins or immunoglobulin-like molecules including IgA, IgD, IgE, IgG and IgM, combinations thereof or fragments thereof. Fragments of antibodies may include, for example, Fab fragments and single chain variable fragments (scFv). An antibody generally comprises heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region (also known as “domains”). In combination, the heavy and the light chain variable regions, also called the “Fab region,” specifically bind to a given antigen. Light and heavy chain variable regions contain a “framework” region interrupted by three hypervariable regions, also called “complementarity-determining regions” or “CDRs.” The extent of the framework region and CDRs has been defined (see Kabat et al., Sequences of Proteins of Immunological Interest, U.S. Department of Health and Human Services, 1991). The Kabat database is now maintained online. The sequences of the framework regions of different light or heavy chains are relatively conserved within a species, and framework regions act to form a scaffold that provides for positioning the CDRs in correct orientation by inter-chain, non-covalent interactions.

The CDRs are primarily responsible for binding to an epitope on an antigen. The CDRs of each chain are typically referred to as CDR1, CDR2, and CDR3, numbered sequentially starting from the N-terminus, and are also typically identified by the chain in which the particular CDR is located. Thus, a HCDR3 is located in the variable domain of the heavy chain of the antibody in which it is found, whereas a LCDR1 is the CDR1 from the variable domain of the light chain of the antibody in which it is found. An antibody that binds IL-31RA will have a specific V_{H} region and the V_{L} region sequence, and thus specific CDR sequences. Antibodies with different specificities generally have different CDRs. Although it is the CDRs that vary from antibody to antibody, only a limited number of amino acid positions within the CDRs are directly involved in antigen binding. These positions within the CDRs are called specificity determining residues (SDRs).

The Fc fragment region (Fc) of an antibody plays a role in modulating immune cell activity. The Fc region functions to guarantee that each antibody generates an appropriate immune response for a given antigen, by binding to a specific class of proteins found on certain cells, such as B lymphocytes, follicular dendritic cells, natural killer cells, macrophages, neutrophils, etc. and are called “Fc receptors.” Because the constant domains of the heavy chains make up the Fc region of an antibody, the classes of heavy chain in antibodies determine their class effects. The heavy chains in antibodies include alpha, gamma, delta, epsilon, and mu, and correlate to the antibody's isotypes IgA, IgG, IgD, IgE, and IgM, respectively. Thus, different isotypes of antibodies have different class effects due to their different Fc regions binding and activating different types of receptors.

There are four subclasses of IgG, which is the most abundant antibody isotype found in human serum. The four subclasses, IgG1, IgG2, IgG3, and IgG4, which are highly conserved. The amino acid sequence of the constant regions of these peptides are known in the art, e.g., see Rutishauser, U. et al. (1968) “Amino acid sequence of the Fc region of a human gamma G-immunoglobulin” PNAS 61(4):1414-1421; Shinoda et al. (1981) “Complete amino acid sequence of the Fc region of a human delta chain” PNAS 78(2):785-789; and Robinson et al. (1980) “Complete amino acid sequence of a mouse immunoglobulin alpha chain (MOPC 511)” PNAS 77(8):4909-4913.

All therapeutic antibodies, for the purposes of the disclosed methods and pharmaceutical uses, are antibodies or fragments thereof that bind to IL-31RA, but the specific anti-IL-31RA antibody is not limited. Nemolizumab is a preferred anti-IL-31RA antibody, but other anti-IL-31RA antibodies can be used as well. A therapeutic antibody suitable for use in the disclosed methods and pharmaceutical uses may be human, humanized, or chimeric, and it may be an IgA, IgG (i.e., IgG1, IgG2, IgG3, and IgG4), IgD, IgE, or IgM.

Nemolizumab is a humanized monoclonal antibody that binds to IL-31RA. Nemolizumab is annotated as follows: immunoglobulin G2-kappa, anti-[Homo sapiens IL31RA (interleukin 31 receptor subunit alpha)], humanized monoclonal antibody; gamma2 heavy chain (1-445) [humanized VH (Homo sapiens IGHV1-2*02 (83.70%)-(IGHD)-IGHJ5*01) [8.8.14] (1-121)-Homo sapiens IGHG2*01 (CH1 C10>S (135), R12>K (137), E16>G (141), S17>G (142) (122-219), hinge C4>S (223) (220-231), CH2 H30>Q (268) (232-340), CH3 R11>Q (355), Q98>E (419) (341-445)) (122-445)], (224-214′)-disulfide with kappa light chain (1′-214′) [humanized V-KAPPA (Homo sapiens IGKV1-39*01 (82.10%)-IGKJ4*01) [6.3.9] (1′-107′)-Homo sapiens IGKC*01 (108′-214′)]; dimer (227-227″:230-230″)-bisdisulfide. Nemolizumab has disulfide bridges at the following locations: Intra-H (C23-C104) 22-96 148-204 261-321 367-425 22″-96″ 148″-204″ 261″-321″ 367″-425″; Intra-L (C23-C104) 23′-88′ 134′-194′ 23′″-88′″ 134′″-194′″; Inter-H-L (h 5-CL 126) 224-214′ 224″-214′″; Inter-H-H (h 8, h 11) 227-227″ 230-230″. Nemolizumab has N-glycosylation sites at the following locations: H CH2 N84.4: 297, 297″. Nemolizumab lacks H chain C-terminal glycine and lysine (CHS G1>del, K2>del).

(SEQ ID NO: 5)
QVQLVQSGAEVKKPGASVKVSCKASGYTFTGYIMNWVRQAPGQGLEWMG
LINPYNGGTDYNPQFQDRVTITADKSTSTAYMELSSLRSEDTAVYYCAR
DGYDDGPYTLETWGQGTLVTVSSASTKGPSVFPLAPSSKSTSGGTAALG
CLVKDYFPEPVTVSWNSGALTSGVHTFPAVLQSSGLYSLSSVVTVPSSN
FGTQTYTCNVDHKPSNTKVDKTVERKSCVECPPCPAPPVAGPSVFLFPP
KPKDTLMISRTPEVTCVVVDVSQEDPEVQFNWYVDGVEVHNAKTKPREE
QFNSTFRVVSVLTVVHQDWLNGKEYKCKVSNKGLPAPIEKTISKTKGQP
REPQVYTLPPSQEEMTKNQVSLTCLVKGFYPSDIAVEWESNGQPENNYK
TTPPMLDSDGSFFLYSKLTVDKSRWQEGNVFSCSVMHEALHNHYTQKSL
SLSP.

Nemolizumab comprises the following light chain amino acid sequence:

(SEQ ID NO: 6)
DIQMTQSPSSLSASVGDRVTITCQASEDIYSFVAWYQQKPGKAPKLLIY
NAQTEAQGVPSRFSGSGSGTDFTLTISSLQPEDFATYYCQHHYDSPLTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

The heavy chain variable region of nemolizumab comprises the amino acid sequence:

(SEQ ID NO: 7)
QVQLVQSGAEVKKPGASVKVSCKASGYTFTGYIMNWVRQAPGQGLEWMG
LINPYNGGTDYNPQFQDRVTITADKSTSTAYMELSSLRSEDTAVYYCAR
DGYDDGPYTLETWGQGTLVTVSS.

The HCDR1 of nemolizumab comprises the amino acid sequence GYIMN (SEQ ID NO: 8), the HCDR2 comprises the amino acid sequence LINPYNGGTDYNPQFQD (SEQ ID NO: 9), and the HCDR3 comprises the amino acid sequence DGYDDGPYTLET (SEQ ID NO: 10).

The light chain variable region of nemolizumab comprises the amino acid sequence:

(SEQ ID NO: 11)
DIQMTQSPSSLSASVGDRVTITCQASEDIYSFVAWYQQKPGKAPKLLIY
NAQTEAQGVPSRFSGSGSGTDFTLTISSLQPEDFATYYCQHHYDSPLTF
GGGTKVEIKR.

The LCDR1 of nemolizumab comprises the amino acid sequence QASEDIYSFVA (SEQ ID NO: 12), the LCDR2 comprises the amino acid sequence NAQTEAQ (SEQ ID NO: 13), and the LCDR3 comprises the amino acid sequence QHHYDSPLT (SEQ ID NO: 14).

For the purposes of this disclosure “variant antibodies” or “variant” of nemolizumab may include, but are not limited to: (i) antibodies with heavy chains comprising at least 55%, at least 65%, at least 70%, at least 75%, at least 80%, at least 85%, at least 90%, at least 95%, at least 97%, at least 98%, at least 99%, or 100% amino acid sequence identity to nemolizumab's heavy chain sequence, (ii) antibodies with light chains comprising at least 55%, at least 65%, at least 70%, at least 75%, at least 80%, at least 85%, at least 90%, at least 95%, at least 97%, at least 98%, at least 99%, or 100% amino acid sequence identity to nemolizumab's light chain sequence, (iii) antibodies with variable regions comprising at least 55%, at least 65%, at least 70%, at least 75%, at least 80%, at least 85%, at least 90%, at least 95%, at least 97%, at least 98%, at least 99%, or 100% amino acid sequence identity to nemolizumab's variable region sequences, (iv) antibodies with CDRs comprising at least 55%, at least 65%, at least 70%, at least 75%, at least 80%, at least 85%, at least 90%, at least 95%, at least 97%, at least 98%, at least 99%, or 100% amino acid sequence identity to nemolizumab's CDR sequences, and (v) combinations thereof. For example, suitable variants include immunoglobulins or immunoglobulin-like molecules with the same or substantially similar heavy and light chain amino acid sequences as nemolizumab. Other suitable therapeutic antibodies may bind to the same isoform of IL-31RA as nemolizumab (e.g., IL31-RAv3), optionally the same epitope of IL-31RA, block or neutralize IL-31RA, or combinations thereof. Additional exemplary therapeutic antibodies are described, for example, in WO 2010/064697.

Variants of nemolizumab and suitable therapeutic antibodies may be monoclonal or polyclonal antibodies. Such monoclonal antibodies having IL31-RA-binding and/or neutralizing activity can be obtained, for example, by the following procedure: anti-IL31-RA monoclonal antibodies are prepared by using as an antigen IL31-RA or a fragment thereof that is derived from a mammal such as human or mouse by known methods, and then antibodies having IL31-RA-binding and/or neutralizing activity are selected from the thus obtained anti-IL31-RA monoclonal antibodies. Specifically, a desired antigen or cells expressing the desired antigen are used as a sensitizing antigen for immunization according to conventional immunization methods. Anti-IL31-RA monoclonal antibodies can be prepared by fusing the obtained immune cells with known parental cells using conventional cell fusion methods, and screening them for monoclonal antibody-producing cells (hybridomas) by conventional screening methods. Animals to be immunized include, for example, mammals such as mice, rats, rabbits, sheep, monkeys, goats, donkeys, cows, horses, and pigs. The antigen can be prepared using the known IL31-RA gene sequence according to known methods, for example, by methods using baculovirus (for example, WO 98/46777). Variants of nemolizumab and suitable therapeutic antibodies may also include intrabodies, peptibodies, nanobodies, single domain antibodies, multi-specific antibodies (e.g., bispecific antibodies, diabodies, triabodies, tetrabodies, tandem di-scFV, tandem tri-scFv), darpins, heavy chain monomers, heavy chain dimers, or single-domain antibodies (i.e., a V_{H}H fragment or a “camelid-like” antibody), any of which may be derived from the sequence and/or binding domain of nemolizumab.

Hybridomas can be prepared, for example, according to the method of Milstein et al. (Kohler, G. And Milstein, C., Methods Enzymol. (1981) 73: 3-46). When the immunogenicity of an antigen is low, immunization may be performed after linking the antigen with a macromolecule having immunogenicity, such as albumin. Antigens used to prepare monoclonal antibodies that have a binding and/or neutralizing activity against human IL31-RA are not particularly limited, as long as they enable preparation of antibodies that have a binding and/or neutralizing activity against human IL31-RA. For example, it is known that there are a number of variants of human IL31-RA, and any variant may be used as an immunogen as long as it enables preparation of antibodies that have a binding and/or neutralizing activity against human IL31-RA. Alternatively, under the same condition, a peptide fragment of IL31-RA or a protein in which artificial mutations have been introduced into the natural IL31-RA sequence may be used as an immunogen. Human IL31-RA.3 is one of preferred immunogens in preparing antibodies that have an activity of binding and/or neutralizing IL31-RA in the present disclosure.

The IL31-RA-binding activity of therapeutic antibodies can be determined by methods known to those skilled in the art. Methods for determining the antigen-binding activity of an antibody include, for example, ELISA (enzyme-linked immunosorbent assay), EIA (enzyme immunoassay), RIA (radioimmunoassay), and fluorescent antibody method. For example, when enzyme immunoassay is used, antibody-containing samples, such as purified antibodies and culture supernatants of antibody-producing cells, are added to antigen-coated plates. A secondary antibody labeled with an enzyme, such as alkaline phosphatase, is added and the plates are incubated. After washing, an enzyme substrate, such as p-nitrophenyl phosphate, is added, and the absorbance is measured to evaluate the antigen-binding activity. The binding and/or neutralizing activity of a therapeutic antibody against IL31-RA can be measured, for example, by observing the effect of suppressing the growth of the IL-31-dependent cell line. For example, the activity of a purified mouse IL-31 antibody can be assayed by assessing the IL-31-dependent growth of Ba/F3 cells transfected with mouse IL-31 receptor a and mouse OSMR genes.

Any of the anti-IL31RA antibodies (i.e. “therapeutic antibodies”) disclosed herein, including nemolizumab and fragments or variants thereof, can be used for treating and/or preventing AD and achieving the disclosed therapeutic endpoints. Optimal doses and routes of administration may vary.

### V. Pharmaceutical Compositions

Provided herein are pharmaceutical compositions for use in the treatment or prevention of atopic dermatitis (AD), including skin lesions or nodules or pruritus caused by AD. The pharmaceutical compositions comprise an anti-IL31RA antibody (i.e. “therapeutic antibody”), such as nemolizumab or a fragment or variant thereof, as an active ingredient. For example, in one aspect, the present disclosure provides a pharmaceutical composition for use in the treatment or prevention of AD in a subject, comprising an anti-IL31RA antibody (e.g., nemolizumab or a fragment or variant thereof), as an active ingredient, wherein the subject expresses in his/her skin at least one, at least two, at least three, at least four, at least five, or all six biomarker(s) selected from CCL20, CCL22, CCL27, VEGF, IL1RA, and CCL18 at an expression level that is higher than a reference level. The reference level may correspond to the level of expression for each biomarker in a skin sample from an individual that does not have AD or, alternatively, in a skin sample from the subject that does not include a lesion or pruritus (i.e., non-lesional skin). In some embodiments, the sample obtained from the subject suspected with AD is a skin sample, which optionally comprises a lesion. For the purposes of the foregoing aspects, a skin sample may comprise, consist of, or consist essentially of stratum corneum.

The present disclosure likewise provides a pharmaceutical composition for use in the treatment or prevention of AD in a subject, comprising an anti-IL31RA antibody (e.g., nemolizumab or a fragment or variant thereof), as an active ingredient, wherein the subject expresses in his/her skin at least one, at least two, at least three, at least four, at least five, or all six biomarker(s) selected from CCL20, CCL22, CCL27, VEGF, IL1RA, and CCL18 at an expression level that is higher than a reference level, and wherein the subject is diagnosed as having AD, by detecting in a sample obtained from a subject suspected of having AD the expression level of at least one of CCL20, CCL22, CCL27, VEGF, IL1RA, and/or CCL18, and optionally comparing the expression level of the biomarkers to a reference level. The reference level may correspond to the level of expression for each biomarker in a skin sample from an individual that does not have AD or, alternatively, in a skin sample from the subject that does not include a lesion or pruritus (i.e., non-lesional skin). In some embodiments, the sample obtained from the subject suspected with AD is a skin sample, which optionally comprises a lesion. For the purposes of the foregoing aspects, a skin sample may comprise, consist of, or consist essentially of stratum corneum.

For the purposes of the foregoing pharmaceutical compositions, expression of CCL18 in the lesional skin of a subject with AD (or suspected of having AD) may be about 300 ppm (when calculated as a ratio of biomarker protein to total protein in a sample), about 310 ppm, about 320 ppm, about 330 ppm, about 340 ppm, about 350 ppm, about 360 ppm, about 370 ppm, about 380 ppm, about 390 ppm, about 400 ppm, or more. Additionally or alternatively, expression of CCL18 in the lesional skin of a subject with AD (or suspected of having AD) may be at least 300 ppm (when calculated as a ratio of biomarker protein to total protein in a sample), at least 310 ppm, at least 320 ppm, at least 330 ppm, at least 340 ppm, at least 350 ppm, at least 360 ppm, at least 370 ppm, at least 380 ppm, at least 390 ppm, at least 400 ppm, or more. Additionally or alternatively, expression of CCL18 in the lesional skin of a subject with AD (or suspected of having AD) may be about 1.5-fold, about 2-fold, about 2.1-fold, about 2.2-fold, about 2.3-fold, about 2.4-fold, about 2.5-fold, about 2.6-fold, about 2.7-fold, about 2.8-fold, about 2.9-fold, or about 3-fold or more than the expression level of CCL18 in a reference sample (e.g., non-lesional skin of the subject or skin from a subject without AD). Additionally or alternatively, expression of CCL18 in the lesional skin of a subject with AD (or suspected of having AD) may be at least 1.5-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, or at least 3-fold or more than the expression level of CCL18 in a reference sample (e.g., non-lesional skin of the subject or skin from a subject without AD). Additionally or alternatively, expression of CCL18 in the lesional skin of a subject with AD (or suspected of having AD) may be about 150%, about 200%, about 210%, about 220%, about 230%, about 240%, about 250%, about 260%, about 270%, about 280%, about 290%, or about 300% or more of the expression level of CCL18 in a reference sample (e.g., non-lesional skin of the subject or skin from a subject without AD). Additionally or alternatively, expression of CCL18 in the lesional skin of a subject with AD (or suspected of having AD) may be at least 150%, at least 200%, at least 210%, at least 220%, at least 230%, at least 240%, at least 250%, at least 260%, at least 270%, at least 280%, at least 290%, or at least 300% or more of the expression level of CCL18 in a reference sample (e.g., non-lesional skin of the subject or skin from a subject without AD).

For the purposes of the foregoing aspects, expression of CCL20 in the lesional skin of a subject with AD (or suspected of having AD) may be about 15.5 ppm (when calculated as a ratio of biomarker protein to total protein in a sample), about 15.6 ppm, about 15.7 ppm, about 15.8 ppm, about 15.9 ppm, about 16 ppm, about 16.1 ppm, about 16.2 ppm, about 16.3 ppm, about 16.4 ppm, about 16.5 ppm, about 16.6 ppm, about 16.7 ppm, about 16.8 ppm, about 16.9 ppm, about 17 ppm or more. Additionally or alternatively, expression of CCL20 in the lesional skin of a subject with AD (or suspected of having AD) may be at least 15.5 ppm (when calculated as a ratio of biomarker protein to total protein in a sample), at least 15.6 ppm, at least 15.7 ppm, at least 15.8 ppm, at least 15.9 ppm, at least 16 ppm, at least 16.1 ppm, at least 16.2 ppm, at least 16.3 ppm, at least 16.4 ppm, at least 16.5 ppm, at least 16.6 ppm, at least 16.7 ppm, at least 16.8 ppm, at least 16.9 ppm, at least 17 ppm or more. Additionally or alternatively, expression of CCL20 in the lesional skin of a subject with AD (or suspected of having AD) may be about 1.1-fold, about 1.2-fold, about 1.3-fold, about 1.4-fold, about 1.5-fold, about 1.6-fold, about 1.7-fold, about 1.8-fold, about 1.9-fold, about 2-fold or more than the expression level of CCL20 in a reference sample (e.g., non-lesional skin of the subject or skin from a subject without AD). Additionally or alternatively, expression of CCL20 in the lesional skin of a subject with AD (or suspected of having AD) may be at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold or more than the expression level of CCL20 in a reference sample (e.g., non-lesional skin of the subject or skin from a subject without AD). Additionally or alternatively, expression of CCL20 in the lesional skin of a subject with AD (or suspected of having AD) may be about 110%, about 120%, about 125%, about 130%, about 135%, about 140%, about 145%, about 150%, about 155%, about 160%, about 165%, about 170%, or about 175% or more of the expression level of CCL20 in a reference sample (e.g., non-lesional skin of the subject or skin from a subject without AD). Additionally or alternatively, expression of CCL20 in the lesional skin of a subject with AD (or suspected of having AD) may be at least 110%, at least 120%, at least 125%, at least 130%, at least 135%, at least 140%, at least 145%, at least 150%, at least 155%, at least 160%, at least 165%, at least 170%, or at least 175% or more of the expression level of CCL20 in a reference sample (e.g., non-lesional skin of the subject or skin from a subject without AD).

For the purposes of the foregoing pharmaceutical compositions, expression of CCL22 in the lesional skin of a subject with AD (or suspected of having AD) may be about 1550 ppm (when calculated as a ratio of biomarker protein to total protein in a sample), about 1560 ppm, about 1570 ppm, about 1580 ppm, about 1590 ppm, about 1600 ppm, about 1610 ppm, about 1620 ppm, about 1630 ppm, about 1640 ppm, about 1650 ppm, about 1660 ppm, about 1670 ppm, about 1680 ppm, about 1690 ppm, about 1700 ppm or more. Additionally or alternatively, expression of CCL22 in the lesional skin of a subject with AD (or suspected of having AD) may be at least 1550 ppm (when calculated as a ratio of biomarker protein to total protein in a sample), at least 1560 ppm, at least 1570 ppm, at least 1580 ppm, at least 1590 ppm, at least 1600 ppm, at least 1610 ppm, at least 1620 ppm, at least 1630 ppm, at least 1640 ppm, at least 1650 ppm, at least 1660 ppm, at least 1670 ppm, at least 1680 ppm, at least 1690 ppm, at least 1700 ppm or more. Additionally or alternatively, expression of CCL22 in the lesional skin of a subject with AD (or suspected of having AD) may be about 1.01-fold, about 1.02-fold, about 1.03-fold, about 1.04-fold, about 1.05-fold, about 1.06-fold, about 1.07-fold, about 1.08-fold, about 1.09-fold, about 1.1-fold, about 1.2-fold, about 1.3-fold, about 1.4-fold, about 1.5-fold, about 1.6-fold, about 1.7-fold, about 1.8-fold, about 1.9-fold, about 2-fold or more than the expression level of CCL22 in a reference sample (e.g., non-lesional skin of the subject or skin from a subject without AD) or the expression level may be about the same between the lesional skin and the reference sample. Additionally or alternatively, expression of CCL22 in the lesional skin of a subject with AD (or suspected of having AD) may be at least 1.01-fold, at least 1.02-fold, at least 1.03-fold, at least 1.04-fold, at least 1.05-fold, at least 1.06-fold, at least 1.07-fold, at least 1.08-fold, at least 1.09-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold or more than the expression level of CCL22 in a reference sample (e.g., non-lesional skin of the subject or skin from a subject without AD) or the expression level may be about the same between the lesional skin and the reference sample. Additionally or alternatively, expression of CCL22 in the lesional skin of a subject with AD (or suspected of having AD) may be about 101%, about 102%, about 103%, about 104%, about 105%, about 106%, about 107%, about 108%, about 109%, about 110%, about 120%, about 125%, about 130%, about 135%, about 140%, about 145%, about 150%, about 155%, about 160%, about 165%, about 170%, or about 175% or more of the expression level of CCL22 in a reference sample (e.g., non-lesional skin of the subject or skin from a subject without AD). Additionally or alternatively, expression of CCL22 in the lesional skin of a subject with AD (or suspected of having AD) may be at least 101%, at least 102%, at least 103%, at least 104%, at least 105%, at least 106%, at least 107%, at least 108%, at least 109%, at least 110%, at least 120%, at least 125%, at least 130%, at least 135%, at least 140%, at least 145%, at least 150%, at least 155%, at least 160%, at least 165%, at least 170%, or at least 175% or more of the expression level of CCL22 in a reference sample (e.g., non-lesional skin of the subject or skin from a subject without AD).

For the purposes of the foregoing pharmaceutical compositions, expression of CCL27 in the lesional skin of a subject with AD (or suspected of having AD) may be about 50 ppm (when calculated as a ratio of biomarker protein to total protein in a sample), about 51 ppm, about 52 ppm, about 53 ppm, about 54 ppm, about 55 ppm, about 56 ppm, about 57 ppm, about 58 ppm, about 59 ppm, about 60 ppm, about 61 ppm, about 62 ppm, about 63 ppm, about 64 ppm, about 65 ppm, about 66 ppm, about 67 ppm, about 68 ppm, about 69 ppm, about 70 ppm, about 71 ppm, about 72 ppm, about 73 ppm, about 74 ppm, about 75 ppm or more. Additionally or alternatively, expression of CCL27 in the lesional skin of a subject with AD (or suspected of having AD) may be at least 50 ppm (when calculated as a ratio of biomarker protein to total protein in a sample), at least 51 ppm, at least 52 ppm, at least 53 ppm, at least 54 ppm, at least 55 ppm, at least 56 ppm, at least 57 ppm, at least 58 ppm, at least 59 ppm, at least 60 ppm, at least 61 ppm, at least 62 ppm, at least 63 ppm, at least 64 ppm, at least 65 ppm, at least 66 ppm, at least 67 ppm, at least 68 ppm, at least 69 ppm, at least 70 ppm, at least 71 ppm, at least 72 ppm, at least 73 ppm, at least 74 ppm, at least 75 ppm or more. Additionally or alternatively, expression of CCL27 in the lesional skin of a subject with AD (or suspected of having AD) may be about 1.5-fold, about 2-fold, about 2.1-fold, about 2.2-fold, about 2.3-fold, about 2.4-fold, about 2.5-fold, about 2.6-fold, about 2.7-fold, about 2.8-fold, about 2.9-fold, or about 3-fold or more than the expression level of CCL27 in a reference sample (e.g., non-lesional skin of the subject or skin from a subject without AD). Additionally or alternatively, expression of CCL27 in the lesional skin of a subject with AD (or suspected of having AD) may be at least 1.5-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, or at least 3-fold or more than the expression level of CCL27 in a reference sample (e.g., non-lesional skin of the subject or skin from a subject without AD). Additionally or alternatively, expression of CCL27 in the lesional skin of a subject with AD (or suspected of having AD) may be about 150%, about 175%, about 200%, about 210%, about 220%, about 225% about 230%, about 240%, about 250%, about 260%, about 270%, about 280%, about 290%, or about 300% or more of the expression level of CCL27 in a reference sample (e.g., non-lesional skin of the subject or skin from a subject without AD). Additionally or alternatively, expression of CCL27 in the lesional skin of a subject with AD (or suspected of having AD) may be at least 150%, at least 175%, at least 200%, at least 210%, at least 220%, at least 225%, at least 230%, at least 240%, at least 250%, at least 260%, at least 270%, at least 280%, at least 290%, or at least 300% or more of the expression level of CCL27 in a reference sample (e.g., non-lesional skin of the subject or skin from a subject without AD).

For the purposes of the foregoing pharmaceutical compositions, expression of VEGF in the lesional skin of a subject with AD (or suspected of having AD) may be about 150 ppm (when calculated as a ratio of biomarker protein to total protein in a sample), about 151 ppm, about 152 ppm, about 153 ppm, about 154 ppm, about 155 ppm, about 156 ppm, about 157 ppm, about 158 ppm, about 159 ppm, about 160 ppm, about 161 ppm, about 162 ppm, about 163 ppm, about 164 ppm, about 165 ppm, about 166 ppm, about 167 ppm, about 168 ppm, about 169 ppm, about 170 ppm, about 171 ppm, about 172 ppm, about 173 ppm, about 174 ppm, about 175 ppm or more. Additionally or alternatively, expression of CCL27 in the lesional skin of a subject with AD (or suspected of having AD) may be at least 150 ppm (when calculated as a ratio of biomarker protein to total protein in a sample), at least 151 ppm, at least 152 ppm, at least 153 ppm, at least 154 ppm, at least 155 ppm, at least 156 ppm, at least 157 ppm, at least 158 ppm, at least 159 ppm, at least 160 ppm, at least 161 ppm, at least 162 ppm, at least 163 ppm, at least 164 ppm, at least 165 ppm, at least 166 ppm, at least 167 ppm, at least 168 ppm, at least 169 ppm, at least 170 ppm, at least 171 ppm, at least 172 ppm, at least 173 ppm, at least 174 ppm, at least 75 ppm or more. Additionally or alternatively, expression of VEGF in the lesional skin of a subject with AD (or suspected of having AD) may be about 1.01-fold, about 1.02-fold, about 1.03-fold, about 1.04-fold, about 1.05-fold, about 1.06-fold, about 1.07-fold, about 1.08-fold, about 1.09-fold, about 1.1-fold, about 1.2-fold, about 1.3-fold, about 1.4-fold, about 1.5-fold, about 1.6-fold, about 1.7-fold, about 1.8-fold, about 1.9-fold, about 2-fold or more than the expression level of VEGF in a reference sample (e.g., non-lesional skin of the subject or skin from a subject without AD) or the expression level may be about the same between the lesional skin and the reference sample. Additionally or alternatively, expression of VEGF in the lesional skin of a subject with AD (or suspected of having AD) may be at least 1.01-fold, at least 1.02-fold, at least 1.03-fold, at least 1.04-fold, at least 1.05-fold, at least 1.06-fold, at least 1.07-fold, at least 1.08-fold, at least 1.09-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold or more than the expression level of VEGF in a reference sample (e.g., non-lesional skin of the subject or skin from a subject without AD) or the expression level may be about the same between the lesional skin and the reference sample. Additionally or alternatively, expression of VEGF in the lesional skin of a subject with AD (or suspected of having AD) may be about 101%, about 102%, about 103%, about 104%, about 105%, about 106%, about 107%, about 108%, about 109%, about 110%, about 120%, about 125%, about 130%, about 135%, about 140%, about 145%, about 150%, about 155%, about 160%, about 165%, about 170%, or about 175% or more of the expression level of VEGF in a reference sample (e.g., non-lesional skin of the subject or skin from a subject without AD). Additionally or alternatively, expression of VEGF in the lesional skin of a subject with AD (or suspected of having AD) may be at least 101%, at least 102%, at least 103%, at least 104%, at least 105%, at least 106%, at least 107%, at least 108%, at least 109%, at least 110%, at least 120%, at least 125%, at least 130%, at least 135%, at least 140%, at least 145%, at least 150%, at least 155%, at least 160%, at least 165%, at least 170%, or at least 175% or more of the expression level of VEGF in a reference sample (e.g., non-lesional skin of the subject or skin from a subject without AD).

For the purposes of the foregoing pharmaceutical compositions, expression of IL1RA in the lesional skin of a subject with AD (or suspected of having AD) may be about 207000 ppm (when calculated as a ratio of biomarker protein to total protein in a sample), about 207500 ppm, about 208000 ppm, about 208500 ppm, about 209000 ppm, about 209500 ppm, about 210000 ppm, about 210500 ppm, about 211000 ppm, about 211500 ppm, about 212000 ppm, about 212500 ppm, about 213000 ppm, about 213500 ppm, about 214000 ppm, about 214500 ppm, about 215000 ppm, about 215500 ppm, about 216000 ppm, about 216500 ppm, about 217000 ppm, about 217500 ppm, about 218000 ppm, about 218500 ppm, about 219000 ppm, about 219500, about 220000, about 221000, about 222000, about 223000, about 224000, about 225000, about 226000, about 227000, about 228000, about 229000, about 230000, about 231000, about 232000, about 233000, about 234000, about 235000 ppm or more. Additionally or alternatively, expression of CCL27 in the lesional skin of a subject with AD (or suspected of having AD) may be at least 207000 ppm (when calculated as a ratio of biomarker protein to total protein in a sample), at least 207500 ppm, at least 208000 ppm, at least 208500 at least, at least 209000 ppm, at least 209500 ppm, at least 210000 ppm, at least 210500 ppm, at least 211000 ppm, at least 211500 ppm, at least 212000 ppm, at least 212500 ppm, at least 213000 ppm, at least 213500 ppm, at least 214000 ppm, at least 214500 ppm, at least 215000 ppm, at least 215500 ppm, at least 216000 ppm, at least 216500 ppm, at least 217000 ppm, at least 217500 ppm, at least 218000 ppm, at least 218500 ppm, at least 219000 ppm, at least 219500, at least 220000, at least 221000, at least 222000, at least 223000, at least 224000, at least 225000, at least 226000, at least 227000, at least 228000, at least 229000, at least 230000, at least 231000, at least 232000, at least 233000, at least 234000, at least 235000 ppm or more. Additionally or alternatively, expression of IL1RA in the lesional skin of a subject with AD (or suspected of having AD) may be about 1.01-fold, about 1.02-fold, about 1.03-fold, about 1.04-fold, about 1.05-fold, about 1.06-fold, about 1.07-fold, about 1.08-fold, about 1.09-fold, about 1.1-fold, about 1.2-fold, about 1.3-fold, about 1.4-fold, about 1.5-fold, about 1.6-fold, about 1.7-fold, about 1.8-fold, about 1.9-fold, about 2-fold or more than the expression level of IL1RA in a reference sample (e.g., non-lesional skin of the subject or skin from a subject without AD) or the expression level may be about the same between the lesional skin and the reference sample. Additionally or alternatively, expression of IL1RA in the lesional skin of a subject with AD (or suspected of having AD) may be at least 1.01-fold, at least 1.02-fold, at least 1.03-fold, at least 1.04-fold, at least 1.05-fold, at least 1.06-fold, at least 1.07-fold, at least 1.08-fold, at least 1.09-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold or more than the expression level of IL1RA in a reference sample (e.g., non-lesional skin of the subject or skin from a subject without AD) or the expression level may be about the same between the lesional skin and the reference sample. Additionally or alternatively, expression of IL1RA in the lesional skin of a subject with AD (or suspected of having AD) may be about 101%, about 102%, about 103%, about 104%, about 105%, about 106%, about 107%, about 108%, about 109%, about 110%, about 115%, about 120%, about 125%, about 130%, about 135%, about 140%, about 145%, about 150%, about 155%, about 160%, about 165%, about 170%, or about 175% or more of the expression level of IL1RA in a reference sample (e.g., non-lesional skin of the subject or skin from a subject without AD). Additionally or alternatively, expression of IL1RA in the lesional skin of a subject with AD (or suspected of having AD) may be at least 101%, at least 102%, at least 103%, at least 104%, at least 105%, at least 106%, at least 107%, at least 108%, at least 109%, at least 110%, at least 115%, at least 120%, at least 125%, at least 130%, at least 135%, at least 140%, at least 145%, at least 150%, at least 155%, at least 160%, at least 165%, at least 170%, or at least 175% or more of the expression level of IL1RA in a reference sample (e.g., non-lesional skin of the subject or skin from a subject without AD).

Use or administration of the disclosed pharmaceutical compositions may also result in a decrease in expression of CCL20, CCL22, CCL27, VEGF, and/or CCL18, and/or an increase in expression of IL1Ra in the skin (specifically, the lesional skin) of the subject receiving the pharmaceutical composition.

For the purposes of the disclosed pharmaceutical compositions, treatment with an anti-IL-31RA antibody (e.g., nemolizumab) may decrease expression of CCL18 in the lesional skin of a subject with AD (or suspected of having AD) by about 6-fold, about 6.5-fold, about 7-fold, about 7.5-fold, about 8-fold, about 8.5 fold or more. Treatment duration necessary to achieve such a decrease may be 4 weeks, 6 weeks, 8 weeks, 10 weeks, 12 weeks, 14 weeks, 16 weeks, 18 weeks, 20 weeks, or more. Applicable dosing and administration regimens are discussed in more detail below.

For the purposes of the disclosed pharmaceutical compositions, treatment with an anti-IL-31RA antibody (e.g., nemolizumab) may decrease expression of CCL20 in the lesional skin of a subject with AD (or suspected of having AD) by about 2-fold, about 2.25-fold, about 2.5-fold, about 2.75-fold, about 3-fold, about 3.25-fold, about 3.5-fold or more. Treatment duration necessary to achieve such a decrease may be 4 weeks, 6 weeks, 8 weeks, 10 weeks, 12 weeks, 14 weeks, 16 weeks, 18 weeks, 20 weeks, or more. Applicable dosing and administration regimens are discussed in more detail below.

For the purposes of the disclosed pharmaceutical compositions, treatment with an anti-IL-31RA antibody (e.g., nemolizumab) may decrease expression of CCL22 in the lesional skin of a subject with AD (or suspected of having AD) by about 1.1, about 1.2, about 1.3, about 1.4, about 1.5, about 1.6, about 1.7, about 1.8, about 1.9, about 2-fold, about 2.25-fold, about 2.5-fold, about 2.75-fold, about 3-fold, about 3.25-fold, about 3.5-fold or more. Treatment duration necessary to achieve such a decrease may be 4 weeks, 6 weeks, 8 weeks, 10 weeks, 12 weeks, 14 weeks, 16 weeks, 18 weeks, 20 weeks, or more. Applicable dosing and administration regimens are discussed in more detail below.

For the purposes of the disclosed pharmaceutical compositions, treatment with an anti-IL-31RA antibody (e.g., nemolizumab) may decrease expression of CCL27 in the lesional skin of a subject with AD (or suspected of having AD) by about 1.5-fold, about 1.75-fold, about 2-fold, about 2.25-fold, about 2.5-fold, about 2.75 fold, about 3-fold, about 3.25-fold, about 3.5-fold or more. Treatment duration necessary to achieve such a decrease may be 4 weeks, 6 weeks, 8 weeks, 10 weeks, 12 weeks, 14 weeks, 16 weeks, 18 weeks, 20 weeks, or more. Applicable dosing and administration regimens are discussed in more detail below.

For the purposes of the disclosed pharmaceutical compositions, treatment with an anti-IL-31RA antibody (e.g., nemolizumab) may decrease expression of VEGF in the lesional skin of a subject with AD (or suspected of having AD) by about 1.1, about 1.2, about 1.3, about 1.4, about 1.5, about 1.6, about 1.7, about 1.8, about 1.9, about 2-fold, about 2.25-fold, about 2.5-fold, about 2.75-fold, about 3-fold, about 3.25-fold, about 3.5-fold or more. Treatment duration necessary to achieve such a decrease may be 4 weeks, 6 weeks, 8 weeks, 10 weeks, 12 weeks, 14 weeks, 16 weeks, 18 weeks, 20 weeks, or more. Applicable dosing and administration regimens are discussed in more detail below.

For the purposes of the disclosed pharmaceutical compositions, treatment with an anti-IL-31RA antibody (e.g., nemolizumab) may increase expression of IL1RA in the lesional skin of a subject with AD (or suspected of having AD) by about 1.1, about 1.2, about 1.3, about 1.4, about 1.5, about 1.6, about 1.7, about 1.8, about 1.9, about 2-fold, about 2.25-fold, about 2.5-fold, about 2.75-fold, about 3-fold, about 3.25-fold, about 3.5-fold or more. Treatment duration necessary to achieve such a decrease may be 4 weeks, 6 weeks, 8 weeks, 10 weeks, 12 weeks, 14 weeks, 16 weeks, 18 weeks, 20 weeks, or more. Applicable dosing and administration regimens are discussed in more detail below.

For the purposes of the disclosed pharmaceutical compositions, the other noted biomarkers (e.g., CXCL6, CXCL8, Cystatin, FASL, Galectin, IL11, IL21, SPD, and CCL17) may show similar patterns of overexpression in the lesional skin of a subject with AD (or suspected of having AD) compared to expression levels in a reference sample (e.g., non-lesional skin of the subject or skin from a subject without AD), and CX3CL1 may show decreased expression in the lesional skin of a subject with AD (or suspected of having AD) compared to expression levels in a reference sample (e.g., non-lesional skin of the subject or skin from a subject without AD). Similarly, CXCL6, CXCL8, Cystatin, FASL, Galectin, CCL17, and IL21 may show similar patterns of decreases of expression in the lesional skin of a subject with AD (or suspected of having AD) after treatment with an anti-IL-31RA antibody (e.g., nemolizumab), and CX3CL1, IL11, and SPD may show increases in expression in the lesional skin of a subject with AD (or suspected of having AD) after treatment with an anti-IL-31RA antibody (e.g., nemolizumab).

The phrase “comprise(s) nemolizumab or a fragment or variant thereof as an active ingredient” means comprising nemolizumab or a fragment or variant thereof as at least one of the active ingredients, and does not limit the proportion of the antibody. In addition, the therapeutic agents for AD in the present disclosure may also comprise, in combination with nemolizumab or a fragment or variant thereof or an equivalent thereof, other ingredients that enhance the treatment or prevention of AD. For example, the composition may comprise one or more topical corticosteroid creams or injections, ointments with menthol or phenol to cool and soothe itchy skin, capsaicin cream, oral corticosteroids, selective serotonin reuptake inhibitors (SSRIs), and oral antihistamines.

Pharmaceutical compositions of an anti-IL31RA antibody (e.g., nemolizumab or a fragment or variant thereof or an equivalent thereof) of the present disclosure can be prepared as formulations according to standard methods (see, for example, Remington's Pharmaceutical Science, Mark Publishing Company, Easton, USA). The pharmaceutical compositions generally comprise a carrier and/or additive in addition to the antibody. For example, in some embodiments, the pharmaceutical composition comprises one or more surfactants (for example, PEG and Tween), excipients, antioxidants (for example, ascorbic acid), coloring agents, flavoring agents, preservatives, stabilizers, buffering agents (for example, phosphoric acid, citric acid, and other organic acids), chelating agents (for example, EDTA), suspending agents, isotonizing agents, binders, disintegrators, lubricants, fluidity promoters, corrigents, light anhydrous silicic acid, lactose, crystalline cellulose, mannitol, starch, carmellose calcium, carmellose sodium, hydroxypropylcellulose, hydroxypropylmethylcellulose, polyvinylacetaldiethylaminoacetate, polyvinylpyrrolidone, gelatin, medium chain fatty acid triglyceride, polyoxyethylene hydrogenated castor oil 60, sucrose, carboxymethylcellulose, corn starch, and inorganic salt. In some embodiments, the pharmaceutical composition comprises one or more other low-molecular-weight polypeptides, proteins such as serum albumin, gelatin, and immunoglobulin, and amino acids such as glycine, glutamine, asparagine, arginine, and lysine.

An anti-IL31RA antibody (e.g., nemolizumab or a fragment or variant thereof) may be prepared as an aqueous solution for injection, in which the anti-IL31RA antibody (e.g., nemolizumab or a fragment or variant thereof) may be dissolved in an isotonic solution containing, for example, physiological saline, dextrose, or other excipients or tonifiers (i.e., tonicity agents). The tonifier may include, for example, D-sorbitol, D-mannose, D-mannitol, and sodium chloride. In addition, appropriate solubilizing agents, for example, alcohols (for example, ethanol), polyalcohols (for example, propylene glycols and PEGs), and non-ionic detergents (polysorbate 80 and HCO-50) may be used concomitantly.

In some embodiments, an anti-IL31RA antibody (e.g., nemolizumab or a fragment or variant thereof) may be encapsulated in microcapsules (microcapsules made of hydroxymethylcellulose, gelatin, polymethylmethacrylate, and the like), and made into components of colloidal drug delivery systems (liposomes, albumin microspheres, microemulsions, nano-particles, and nano-capsules) (for example, see “Remington's Pharmaceutical Science 16th edition” &, Oslo Ed. (1980)). Moreover, methods for making sustained-release drugs are known, and these can be applied for nemolizumab or a fragment or variant thereof (Langer et al., J. Biomed. Mater. Res. (1981) 15, 167-277; Langer, Chem. Tech. (1982) 12, 98-105; U.S. Pat. No. 3,773,919; European Patent Application (EP) No. 58,481; Sidman et al., Biopolymers (1983) 22, 547-56; EP 133,988).

The pharmaceutical compositions of the present disclosure can be administered either orally or parenterally, but are preferably administered parenterally. Specifically, the pharmaceutical compositions are administered to patients by injection or percutaneous administration. Injections include, for example, intravenous injections, intramuscular injections, and subcutaneous injections, for systemic or local administration. The pharmaceutical compositions may be given to sites where inflammation and/or itching is to be suppressed, or areas surrounding the sites by local infusion or intramuscular or subcutaneous injection. In some embodiments, the pharmaceutical compositions are administered at the site of one or more skin excoriations, lesions, or nodules, or proximal to the site of one or more skin excoriations, lesions, or nodules.

The administration methods can be properly selected according to the patient's age, weight, and condition. The single-administration dose can be selected, for example, from within the range of 0.0001 to 100 mg of the antibody (e.g., nemolizumab or a fragment or variant thereof) per kg body weight. Alternatively, for example, when the antibody is administered to human patients, the dose of the antibody can be selected from within the range of 0.001 to 1,000 mg/kg body weight. In some embodiments, the composition is formulated to administer a dose containing, for example, about 0.01 to 50 mg/kg, about 0.01 mg/kg to about 0.1 mg/kg, about 0.05 mg/kg to 0.15 mg/kg, about 0.1 mg/kg to about 0.6 mg/kg, about 0.1 mg/kg to about 1 mg/kg, about 0.25 mg/kg to about 0.75 mg/kg, about 0.4 mg/kg to about 0.8 mg/kg, about 0.4 mg/kg to about 1.8 mg/kg, about 0.5 to about 2.5 mg/kg, about 0.8 mg/kg to about 2.2 mg/kg, about 1 mg/kg to about 2.5 mg/kg, about 1 mg/kg to about 3.5 mg/kg, about 1 mg/kg to about 5 mg/kg, about 2 mg/kg to about 4 mg/kg, about 2.5 mg/kg to about 10 mg/kg, about 5 mg/kg to about 10 mg/kg, about 10 mg/kg to about 20 mg/kg, about 10 mg/kg to about 40 mg/kg, about 20 mg/kg to about 50 mg/kg, about 25 mg/kg to about 75 mg/kg, about 50 mg/kg to about 100 mg/kg, or about 100 mg/kg to about 500 mg/kg, or about 100 mg/kg to about 1000 mg/kg body weight of nemolizumab or a fragment or variant thereof. In preferred embodiments, the dose ranges from about 0.01 mg/kg to about 0.1 mg/kg, about 0.1 mg/kg to about 0.5 mg/kg, about 0.5 mg/kg to about 1.5 mg/kg, about 1.5 mg/kg to about 2.5 mg/kg, or about 2.5 mg/kg to about 10 mg/kg. In some embodiments, the dose is about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3 mg/kg, about 3.5 mg/kg, about 4 mg/kg, about 4.5 mg/kg, about 5 mg/kg, about 6 mg/kg, about 7 mg/kg, about 8 mg/kg, about 9 mg/kg, about 10 mg/kg, about 15 mg/kg, about 25 mg/kg, about 50 mg/kg, about 75 mg/kg, about 100 mg/kg, about 500 mg/kg, or about 1,000 mg/kg. In particular embodiments, the effective amount of nemolizumab or a fragment or variant thereof is about 0.1 mg/kg, about 0.5 mg/kg, about 1 mg/kg, about 1.5 mg/kg, about 2 mg/kg, or about 2.5 mg/kg. In a preferred embodiment, the dose is about 0.5 mg/kg.

The present disclosure provides a pharmaceutical composition for use in the treatment or prevention of atopic dermatitis (AD) in a subject, comprising an anti-TL31RA antibody (e.g., nemolizumab or a fragment or variant thereof), as an active ingredient, wherein the subject differentially expresses at least one of CCL20, CCL22, CCL27, VEGF, IL1RA, and CCL18 compared to a reference level of expression for the at least one gene.

Any of the pharmaceutical compositions disclosed herein, including nemolizumab and fragments or variants thereof, can be used for treating and/or preventing AD and achieving the disclosed therapeutic endpoints. Optimal doses and routes of administration may vary.

### VI. Treatment/Prevention of Atopic Dermatitis

The present disclosure provides methods of treating or preventing pruritus in a subject having atopic dermatitis (AD), the method comprising, consisting of, or consisting essentially of administering an anti-IL-31RA antibody (i.e., a “therapeutic antibody”), such as nemolizumab or a fragment or variant thereof to the subject. The disclosed methods may be performed to achieve specific therapeutic endpoints, which are discussed in more detail below. Also disclosed herein are uses of an anti-IL-31RA antibody (i.e., a “therapeutic antibody”), such as nemolizumab or a fragment or variant thereof to the subject for treating or preventing AD and/or achieving the disclosed therapeutic endpoints. Also disclosed herein are anti-IL-31RA antibodies (i.e., a “therapeutic antibodies”), such as nemolizumab or a fragment or variant thereof to the subject for use in treating or preventing AD and/or achieving the disclosed therapeutic endpoints. Additionally, particular subgroups of subjects with AD may be especially suitable for treatment according to the disclosed methods and uses. For example, patients presenting with increased expression of the biomarkers of the present disclosure, such as CCL20, CCL22, CCL27, VEGF, IL1RA, and/or CCL18 were discovered to respond particularly well to treatment with anti-IL-31RA antibodies (e.g., nemolizumab). Similarly, the present disclosure is the first to show that adolescents with moderate to severe AD respond to treatment with anti-IL-31RA antibodies (e.g., nemolizumab. Accordingly, in some embodiments of the disclosed methods and uses the subject being treated may present with increased expression of CCL20, CCL22, CCL27, VEGF, IL1RA, and/or CCL18 in his/her skin; in some embodiments, the subject may be an adolescent; and in some embodiments, the subject may be an adolescent that presents with increased expression of CCL20, CCL22, CCL27, VEGF, IL1RA, and/or CCL18 in his/her skin.

The present disclosure is the first to report a biomarker signature for AD that is able to not only identify and positively diagnose AD, but also identify subjects with AD that will likely respond to treatment with an anti-IL-31RA antibody (e.g., nemolizumab) and track the response of the treatment with an anti-IL-31RA antibody (e.g., nemolizumab).

In one aspect, the present disclosure provides a method of treating or preventing atopic dermatitis (AD) in a subject, comprising administering to a subject with AD an anti-IL-31RA antibody, wherein the subject has skin lesions in which expression of at least one of CCL20, CCL22, CCL27, VEGF, IL1RA, and CCL18 are up-regulated compared to non-lesional skin of an individual without AD. In some embodiments, “preventing” AD can refer to simply reducing the risk of a flare or reducing the risk of development of skin lesions.

In another aspect, the present disclosure provides a method of reducing expression of at least one of CCL20, CCL22, CCL27, VEGF, IL1RA, and CCL18 in a skin lesion of a subject with atopic dermatitis (AD) comprising administering to a subject with AD an anti-IL-31RA antibody, wherein at least one of CCL20, CCL22, CCL27, VEGF, IL1RA, and CCL18 are up-regulated in a skin lesion of the subject compared to non-lesional skin of the subject or an individual without AD, and wherein administration of the anti-IL-31RA antibody reduces the expression of at least one of CCL20, CCL22, CCL27, VEGF, IL1RA, and CCL18 in a skin lesion.

For the purposes of the disclosed uses and methods, one, two, three, four, five, or six of CCL20, CCL22, CCL27, VEGF, IL1RA, and CCL18 may be up-regulated in at least one skin lesion of the subject compared to non-lesional skin of the subject or an individual without AD. In some embodiments, CCL20, CCL22, CCL27, and VEGF are up-regulated in the skin lesion of the subject compared to non-lesional skin of the subject or an individual without AD.

Additionally or alternatively, in some embodiments, the expression of at least one of CCL20, CCL22, CCL27, VEGF, IL1RA, and CCL18 is reduced within 2 weeks, 4 week, 8 weeks, 10 weeks, 12 weeks, 14 weeks, 16 week, 18 weeks, or 20 weeks of the administration of the anti-IL-31RA antibody compared a baseline level of expression in the skin lesion of the subject prior to administration of the anti-IL-31RA antibody. Additionally or alternatively, in some embodiments, expression of at least one of CCL20, CCL22, CCL27, VEGF, IL1RA, and CCL18 is reduced or increased as discussed herein.

For example, expression of CCL18 in the lesional skin of a subject with AD (or suspected of having AD) may be about 300 ppm (when calculated as a ratio of biomarker protein to total protein in a sample), about 310 ppm, about 320 ppm, about 330 ppm, about 340 ppm, about 350 ppm, about 360 ppm, about 370 ppm, about 380 ppm, about 390 ppm, about 400 ppm, or more. Additionally or alternatively, expression of CCL18 in the lesional skin of a subject with AD (or suspected of having AD) may be at least 300 ppm (when calculated as a ratio of biomarker protein to total protein in a sample), at least 310 ppm, at least 320 ppm, at least 330 ppm, at least 340 ppm, at least 350 ppm, at least 360 ppm, at least 370 ppm, at least 380 ppm, at least 390 ppm, at least 400 ppm, or more. Additionally or alternatively, expression of CCL18 in the lesional skin of a subject with AD (or suspected of having AD) may be about 1.5-fold, about 2-fold, about 2.1-fold, about 2.2-fold, about 2.3-fold, about 2.4-fold, about 2.5-fold, about 2.6-fold, about 2.7-fold, about 2.8-fold, about 2.9-fold, or about 3-fold or more than the expression level of CCL18 in a reference sample (e.g., non-lesional skin of the subject or skin from a subject without AD). Additionally or alternatively, expression of CCL18 in the lesional skin of a subject with AD (or suspected of having AD) may be at least 1.5-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, or at least 3-fold or more than the expression level of CCL18 in a reference sample (e.g., non-lesional skin of the subject or skin from a subject without AD). Additionally or alternatively, expression of CCL18 in the lesional skin of a subject with AD (or suspected of having AD) may be about 150%, about 200%, about 210%, about 220%, about 230%, about 240%, about 250%, about 260%, about 270%, about 280%, about 290%, or about 300% or more of the expression level of CCL18 in a reference sample (e.g., non-lesional skin of the subject or skin from a subject without AD). Additionally or alternatively, expression of CCL18 in the lesional skin of a subject with AD (or suspected of having AD) may be at least 150%, at least 200%, at least 210%, at least 220%, at least 230%, at least 240%, at least 250%, at least 260%, at least 270%, at least 280%, at least 290%, or at least 300% or more of the expression level of CCL18 in a reference sample (e.g., non-lesional skin of the subject or skin from a subject without AD).

Expression of CCL20 in the lesional skin of a subject with AD (or suspected of having AD) may be about 15.5 ppm (when calculated as a ratio of biomarker protein to total protein in a sample), about 15.6 ppm, about 15.7 ppm, about 15.8 ppm, about 15.9 ppm, about 16 ppm, about 16.1 ppm, about 16.2 ppm, about 16.3 ppm, about 16.4 ppm, about 16.5 ppm, about 16.6 ppm, about 16.7 ppm, about 16.8 ppm, about 16.9 ppm, about 17 ppm or more. Additionally or alternatively, expression of CCL20 in the lesional skin of a subject with AD (or suspected of having AD) may be at least 15.5 ppm (when calculated as a ratio of biomarker protein to total protein in a sample), at least 15.6 ppm, at least 15.7 ppm, at least 15.8 ppm, at least 15.9 ppm, at least 16 ppm, at least 16.1 ppm, at least 16.2 ppm, at least 16.3 ppm, at least 16.4 ppm, at least 16.5 ppm, at least 16.6 ppm, at least 16.7 ppm, at least 16.8 ppm, at least 16.9 ppm, at least 17 ppm or more. Additionally or alternatively, expression of CCL20 in the lesional skin of a subject with AD (or suspected of having AD) may be about 1.1-fold, about 1.2-fold, about 1.3-fold, about 1.4-fold, about 1.5-fold, about 1.6-fold, about 1.7-fold, about 1.8-fold, about 1.9-fold, about 2-fold or more than the expression level of CCL20 in a reference sample (e.g., non-lesional skin of the subject or skin from a subject without AD). Additionally or alternatively, expression of CCL20 in the lesional skin of a subject with AD (or suspected of having AD) may be at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold or more than the expression level of CCL20 in a reference sample (e.g., non-lesional skin of the subject or skin from a subject without AD). Additionally or alternatively, expression of CCL20 in the lesional skin of a subject with AD (or suspected of having AD) may be about 110%, about 120%, about 125%, about 130%, about 135%, about 140%, about 145%, about 150%, about 155%, about 160%, about 165%, about 170%, or about 175% or more of the expression level of CCL20 in a reference sample (e.g., non-lesional skin of the subject or skin from a subject without AD). Additionally or alternatively, expression of CCL20 in the lesional skin of a subject with AD (or suspected of having AD) may be at least 110%, at least 120%, at least 125%, at least 130%, at least 135%, at least 140%, at least 145%, at least 150%, at least 155%, at least 160%, at least 165%, at least 170%, or at least 175% or more of the expression level of CCL20 in a reference sample (e.g., non-lesional skin of the subject or skin from a subject without AD).

Expression of CCL22 in the lesional skin of a subject with AD (or suspected of having AD) may be about 1550 ppm (when calculated as a ratio of biomarker protein to total protein in a sample), about 1560 ppm, about 1570 ppm, about 1580 ppm, about 1590 ppm, about 1600 ppm, about 1610 ppm, about 1620 ppm, about 1630 ppm, about 1640 ppm, about 1650 ppm, about 1660 ppm, about 1670 ppm, about 1680 ppm, about 1690 ppm, about 1700 ppm or more. Additionally or alternatively, expression of CCL22 in the lesional skin of a subject with AD (or suspected of having AD) may be at least 1550 ppm (when calculated as a ratio of biomarker protein to total protein in a sample), at least 1560 ppm, at least 1570 ppm, at least 1580 ppm, at least 1590 ppm, at least 1600 ppm, at least 1610 ppm, at least 1620 ppm, at least 1630 ppm, at least 1640 ppm, at least 1650 ppm, at least 1660 ppm, at least 1670 ppm, at least 1680 ppm, at least 1690 ppm, at least 1700 ppm or more. Additionally or alternatively, expression of CCL22 in the lesional skin of a subject with AD (or suspected of having AD) may be about 1.01-fold, about 1.02-fold, about 1.03-fold, about 1.04-fold, about 1.05-fold, about 1.06-fold, about 1.07-fold, about 1.08-fold, about 1.09-fold, about 1.1-fold, about 1.2-fold, about 1.3-fold, about 1.4-fold, about 1.5-fold, about 1.6-fold, about 1.7-fold, about 1.8-fold, about 1.9-fold, about 2-fold or more than the expression level of CCL22 in a reference sample (e.g., non-lesional skin of the subject or skin from a subject without AD) or the expression level may be about the same between the lesional skin and the reference sample. Additionally or alternatively, expression of CCL22 in the lesional skin of a subject with AD (or suspected of having AD) may be at least 1.01-fold, at least 1.02-fold, at least 1.03-fold, at least 1.04-fold, at least 1.05-fold, at least 1.06-fold, at least 1.07-fold, at least 1.08-fold, at least 1.09-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold or more than the expression level of CCL22 in a reference sample (e.g., non-lesional skin of the subject or skin from a subject without AD) or the expression level may be about the same between the lesional skin and the reference sample. Additionally or alternatively, expression of CCL22 in the lesional skin of a subject with AD (or suspected of having AD) may be about 101%, about 102%, about 103%, about 104%, about 105%, about 106%, about 107%, about 108%, about 109%, about 110%, about 120%, about 125%, about 130%, about 135%, about 140%, about 145%, about 150%, about 155%, about 160%, about 165%, about 170%, or about 175% or more of the expression level of CCL22 in a reference sample (e.g., non-lesional skin of the subject or skin from a subject without AD). Additionally or alternatively, expression of CCL22 in the lesional skin of a subject with AD (or suspected of having AD) may be at least 101%, at least 102%, at least 103%, at least 104%, at least 105%, at least 106%, at least 107%, at least 108%, at least 109%, at least 110%, at least 120%, at least 125%, at least 130%, at least 135%, at least 140%, at least 145%, at least 150%, at least 155%, at least 160%, at least 165%, at least 170%, or at least 175% or more of the expression level of CCL22 in a reference sample (e.g., non-lesional skin of the subject or skin from a subject without AD).

Expression of CCL27 in the lesional skin of a subject with AD (or suspected of having AD) may be about 50 ppm (when calculated as a ratio of biomarker protein to total protein in a sample), about 51 ppm, about 52 ppm, about 53 ppm, about 54 ppm, about 55 ppm, about 56 ppm, about 57 ppm, about 58 ppm, about 59 ppm, about 60 ppm, about 61 ppm, about 62 ppm, about 63 ppm, about 64 ppm, about 65 ppm, about 66 ppm, about 67 ppm, about 68 ppm, about 69 ppm, about 70 ppm, about 71 ppm, about 72 ppm, about 73 ppm, about 74 ppm, about 75 ppm or more. Additionally or alternatively, expression of CCL27 in the lesional skin of a subject with AD (or suspected of having AD) may be at least 50 ppm (when calculated as a ratio of biomarker protein to total protein in a sample), at least 51 ppm, at least 52 ppm, at least 53 ppm, at least 54 ppm, at least 55 ppm, at least 56 ppm, at least 57 ppm, at least 58 ppm, at least 59 ppm, at least 60 ppm, at least 61 ppm, at least 62 ppm, at least 63 ppm, at least 64 ppm, at least 65 ppm, at least 66 ppm, at least 67 ppm, at least 68 ppm, at least 69 ppm, at least 70 ppm, at least 71 ppm, at least 72 ppm, at least 73 ppm, at least 74 ppm, at least 75 ppm or more. Additionally or alternatively, expression of CCL27 in the lesional skin of a subject with AD (or suspected of having AD) may be about 1.5-fold, about 2-fold, about 2.1-fold, about 2.2-fold, about 2.3-fold, about 2.4-fold, about 2.5-fold, about 2.6-fold, about 2.7-fold, about 2.8-fold, about 2.9-fold, or about 3-fold or more than the expression level of CCL27 in a reference sample (e.g., non-lesional skin of the subject or skin from a subject without AD). Additionally or alternatively, expression of CCL27 in the lesional skin of a subject with AD (or suspected of having AD) may be at least 1.5-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, or at least 3-fold or more than the expression level of CCL27 in a reference sample (e.g., non-lesional skin of the subject or skin from a subject without AD). Additionally or alternatively, expression of CCL27 in the lesional skin of a subject with AD (or suspected of having AD) may be about 150%, about 175%, about 200%, about 210%, about 220%, about 225% about 230%, about 240%, about 250%, about 260%, about 270%, about 280%, about 290%, or about 300% or more of the expression level of CCL27 in a reference sample (e.g., non-lesional skin of the subject or skin from a subject without AD). Additionally or alternatively, expression of CCL27 in the lesional skin of a subject with AD (or suspected of having AD) may be at least 150%, at least 175%, at least 200%, at least 210%, at least 220%, at least 225%, at least 230%, at least 240%, at least 250%, at least 260%, at least 270%, at least 280%, at least 290%, or at least 300% or more of the expression level of CCL27 in a reference sample (e.g., non-lesional skin of the subject or skin from a subject without AD).

Expression of VEGF in the lesional skin of a subject with AD (or suspected of having AD) may be about 150 ppm (when calculated as a ratio of biomarker protein to total protein in a sample), about 151 ppm, about 152 ppm, about 153 ppm, about 154 ppm, about 155 ppm, about 156 ppm, about 157 ppm, about 158 ppm, about 159 ppm, about 160 ppm, about 161 ppm, about 162 ppm, about 163 ppm, about 164 ppm, about 165 ppm, about 166 ppm, about 167 ppm, about 168 ppm, about 169 ppm, about 170 ppm, about 171 ppm, about 172 ppm, about 173 ppm, about 174 ppm, about 175 ppm or more. Additionally or alternatively, expression of CCL27 in the lesional skin of a subject with AD (or suspected of having AD) may be at least 150 ppm (when calculated as a ratio of biomarker protein to total protein in a sample), at least 151 ppm, at least 152 ppm, at least 153 ppm, at least 154 ppm, at least 155 ppm, at least 156 ppm, at least 157 ppm, at least 158 ppm, at least 159 ppm, at least 160 ppm, at least 161 ppm, at least 162 ppm, at least 163 ppm, at least 164 ppm, at least 165 ppm, at least 166 ppm, at least 167 ppm, at least 168 ppm, at least 169 ppm, at least 170 ppm, at least 171 ppm, at least 172 ppm, at least 173 ppm, at least 174 ppm, at least 75 ppm or more. Additionally or alternatively, expression of VEGF in the lesional skin of a subject with AD (or suspected of having AD) may be about 1.01-fold, about 1.02-fold, about 1.03-fold, about 1.04-fold, about 1.05-fold, about 1.06-fold, about 1.07-fold, about 1.08-fold, about 1.09-fold, about 1.1-fold, about 1.2-fold, about 1.3-fold, about 1.4-fold, about 1.5-fold, about 1.6-fold, about 1.7-fold, about 1.8-fold, about 1.9-fold, about 2-fold or more than the expression level of VEGF in a reference sample (e.g., non-lesional skin of the subject or skin from a subject without AD) or the expression level may be about the same between the lesional skin and the reference sample. Additionally or alternatively, expression of VEGF in the lesional skin of a subject with AD (or suspected of having AD) may be at least 1.01-fold, at least 1.02-fold, at least 1.03-fold, at least 1.04-fold, at least 1.05-fold, at least 1.06-fold, at least 1.07-fold, at least 1.08-fold, at least 1.09-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold or more than the expression level of VEGF in a reference sample (e.g., non-lesional skin of the subject or skin from a subject without AD) or the expression level may be about the same between the lesional skin and the reference sample. Additionally or alternatively, expression of VEGF in the lesional skin of a subject with AD (or suspected of having AD) may be about 101%, about 102%, about 103%, about 104%, about 105%, about 106%, about 107%, about 108%, about 109%, about 110%, about 120%, about 125%, about 130%, about 135%, about 140%, about 145%, about 150%, about 155%, about 160%, about 165%, about 170%, or about 175% or more of the expression level of VEGF in a reference sample (e.g., non-lesional skin of the subject or skin from a subject without AD). Additionally or alternatively, expression of VEGF in the lesional skin of a subject with AD (or suspected of having AD) may be at least 101%, at least 102%, at least 103%, at least 104%, at least 105%, at least 106%, at least 107%, at least 108%, at least 109%, at least 110%, at least 120%, at least 125%, at least 130%, at least 135%, at least 140%, at least 145%, at least 150%, at least 155%, at least 160%, at least 165%, at least 170%, or at least 175% or more of the expression level of VEGF in a reference sample (e.g., non-lesional skin of the subject or skin from a subject without AD).

Expression of IL1RA in the lesional skin of a subject with AD (or suspected of having AD) may be about 207000 ppm (when calculated as a ratio of biomarker protein to total protein in a sample), about 207500 ppm, about 208000 ppm, about 208500 ppm, about 209000 ppm, about 209500 ppm, about 210000 ppm, about 210500 ppm, about 211000 ppm, about 211500 ppm, about 212000 ppm, about 212500 ppm, about 213000 ppm, about 213500 ppm, about 214000 ppm, about 214500 ppm, about 215000 ppm, about 215500 ppm, about 216000 ppm, about 216500 ppm, about 217000 ppm, about 217500 ppm, about 218000 ppm, about 218500 ppm, about 219000 ppm, about 219500, about 220000, about 221000, about 222000, about 223000, about 224000, about 225000, about 226000, about 227000, about 228000, about 229000, about 230000, about 231000, about 232000, about 233000, about 234000, about 235000 ppm or more. Additionally or alternatively, expression of CCL27 in the lesional skin of a subject with AD (or suspected of having AD) may be at least 207000 ppm (when calculated as a ratio of biomarker protein to total protein in a sample), at least 207500 ppm, at least 208000 ppm, at least 208500 at least, at least 209000 ppm, at least 209500 ppm, at least 210000 ppm, at least 210500 ppm, at least 211000 ppm, at least 211500 ppm, at least 212000 ppm, at least 212500 ppm, at least 213000 ppm, at least 213500 ppm, at least 214000 ppm, at least 214500 ppm, at least 215000 ppm, at least 215500 ppm, at least 216000 ppm, at least 216500 ppm, at least 217000 ppm, at least 217500 ppm, at least 218000 ppm, at least 218500 ppm, at least 219000 ppm, at least 219500, at least 220000, at least 221000, at least 222000, at least 223000, at least 224000, at least 225000, at least 226000, at least 227000, at least 228000, at least 229000, at least 230000, at least 231000, at least 232000, at least 233000, at least 234000, at least 235000 ppm or more. Additionally or alternatively, expression of IL1RA in the lesional skin of a subject with AD (or suspected of having AD) may be about 1.01-fold, about 1.02-fold, about 1.03-fold, about 1.04-fold, about 1.05-fold, about 1.06-fold, about 1.07-fold, about 1.08-fold, about 1.09-fold, about 1.1-fold, about 1.2-fold, about 1.3-fold, about 1.4-fold, about 1.5-fold, about 1.6-fold, about 1.7-fold, about 1.8-fold, about 1.9-fold, about 2-fold or more than the expression level of IL1RA in a reference sample (e.g., non-lesional skin of the subject or skin from a subject without AD) or the expression level may be about the same between the lesional skin and the reference sample. Additionally or alternatively, expression of IL1RA in the lesional skin of a subject with AD (or suspected of having AD) may be at least 1.01-fold, at least 1.02-fold, at least 1.03-fold, at least 1.04-fold, at least 1.05-fold, at least 1.06-fold, at least 1.07-fold, at least 1.08-fold, at least 1.09-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold or more than the expression level of IL1RA in a reference sample (e.g., non-lesional skin of the subject or skin from a subject without AD) or the expression level may be about the same between the lesional skin and the reference sample. Additionally or alternatively, expression of IL1RA in the lesional skin of a subject with AD (or suspected of having AD) may be about 101%, about 102%, about 103%, about 104%, about 105%, about 106%, about 107%, about 108%, about 109%, about 110%, about 115%, about 120%, about 125%, about 130%, about 135%, about 140%, about 145%, about 150%, about 155%, about 160%, about 165%, about 170%, or about 175% or more of the expression level of IL1RA in a reference sample (e.g., non-lesional skin of the subject or skin from a subject without AD). Additionally or alternatively, expression of IL1RA in the lesional skin of a subject with AD (or suspected of having AD) may be at least 101%, at least 102%, at least 103%, at least 104%, at least 105%, at least 106%, at least 107%, at least 108%, at least 109%, at least 110%, at least 115%, at least 120%, at least 125%, at least 130%, at least 135%, at least 140%, at least 145%, at least 150%, at least 155%, at least 160%, at least 165%, at least 170%, or at least 175% or more of the expression level of IL1RA in a reference sample (e.g., non-lesional skin of the subject or skin from a subject without AD).

In another aspect, the present disclosure provides a method of reducing expression of an inflammatory biomarker in the skin of a subject with atopic dermatitis (AD), comprising administering to a subject with AD an anti-IL-31RA antibody, thereby reducing expression of the inflammatory biomarker in the skin. In some embodiments, the inflammatory biomarker is selected from at least one of CCL20, CCL22, CCL27, VEGF, IL1RA, and CCL18. In some embodiments, the inflammatory biomarker is selected from at least one of CCL20, CCL22, CCL27, and VEGF. In some embodiments, expression of one, two, three, or all four of CCL20, CCL22, CCL27, and VEGF is reduced. In some embodiments, expression of the inflammatory biomarker is reduced in at least one skin lesion of the subject. The reduction of the biomarker can be measured by taking a baseline measurement of expression of the biomarker(s) at a time 0 (i.e., prior to administration of the anti-IL-31RA antibody (e.g., nemolizumab), and comparing this initial measure to a later time point after treatment has commenced, such as 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks or more after treatment has commenced. In some embodiments, treatment with the anti-IL31RA antibody may comprise repeated administration of the antibody once every week, 2 weeks, 3 weeks, 4 week, 5 weeks, or 6 weeks, and the dosing regimen may be “flat” or comprise a loading dose, as discussed in more detail below.

For the purposes of the disclosed methods and uses, administering the anti-IL-31RA antibody (i.e., a “therapeutic antibody”), such as nemolizumab or a fragment or variant thereof to the subject, may be effective to prevent, reduce, ameliorate, or eliminate one or more of symptoms/complications caused by AD including but not limited to pruritus (itching), xerosis (skin dryness), eczematous lesions, and significant sleep disturbances, and/or improve quality of life in a subject with AD. While it is understood that not every subject with AD will respond to treatment, subjects that possess skin lesions with increased expression of CCL20, CCL22, CCL27, VEGF, IL1RA, and/or CCL18 (compared to non-lesional samples of the subject's skin or non-lesional samples of skin of an individual without AD) are likely to response favorably to treatment with an anti-IL31RA antibody, such as nemolizumab.

For the purposes of the disclosed methods and uses, the subject may achieve at least a 66.5% decrease in Eczema Area and Severity Index (EASI) scoring following administration of the anti-IL-31RA antibody. In some embodiments, the decrease in EASI scoring may be at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% or more. Additionally or alternatively, in some embodiments, the subject may achieve at least a 53.3% decrease in body surface area (EASI) of AD involvement following administration of the anti-IL-31RA antibody. In some embodiments, the decrease in body surface area EASI scoring may be at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% or more.

For the purposes of the disclosed methods and uses, the subject may achieve at least a 31.2% decrease in Scoring Atopic Dermatitis (SCORAD) scoring following administration of the anti-IL-31RA antibody. In some embodiments, the decrease in SCORAD scoring may be at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% or more.

For the purposes of the disclosed methods and uses, the subject may achieve at least a 43.2% decrease in peak pruritis numeric rating scale (PP-NRS) scoring following administration of the anti-IL-31RA antibody. In some embodiments, the decrease in PP-NRS scoring may be at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% or more. In some embodiments, the subject achieves at least a 40.9% decrease in average pruritis numeric rating scale (AP NRS) scoring following administration of the anti-IL-31RA antibody. In some embodiments, the decrease in AP NRS scoring may be at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% or more.

Additionally or alternatively, in some embodiments, the subject experiences improved sleep following administration of the anti-IL-31RA antibody. In some embodiments, the subject achieves at least a 53.5% decrease in sleep disturbance numeric rating scale following administration of the anti-IL-31RA antibody. In some embodiments, the decrease in sleep disturbance numeric rating scale may be at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% or more.

Additionally or alternatively, in some embodiments, the subject experiences increased medication-free days following administration of the anti-IL-31RA antibody. For example, the subject may not require any other medication for 1, 2, 3, 4, 5, 6, or 7 days, or 1 week, 2 weeks, 3 weeks, 4 weeks, or more. Additionally or alternatively, in some embodiments, the subject achieves improved dermatology life qualify index (DLQI) or Children's Dermatology Life Quality Index (cDLQI) scoring.

In some embodiments of the methods disclosed herein, the subject is an adult (i.e., 18 years old or older). In some embodiments, the subject is an adolescent, for example, between the ages of 12 and 17 years old. In some embodiments, the subject is less than 12 years old, such as 7-11 years old or 2-6 years old. In some embodiments, the subject is diagnosed of AD. In some embodiments, the subject is suspected of having AD, or at risk of developing AD.

In some embodiments, the anti-IL-31RA antibody is administered subcutaneously. In some embodiments, the anti-IL-31RA antibody is administered intravenously, intramuscularly, intraperitoneally, or otherwise via injection. In some embodiments, the anti-IL-31RA antibody is administered once per week, once every two weeks, once every three weeks, once every four weeks, once every five weeks, once every six weeks, once every seven weeks, or once every eight weeks. In some embodiments, the anti-IL-31RA antibody is administered once every four weeks.

In some embodiments, the anti-IL-31RA antibody is administered at a dose of about 0.01 mg/kg to about 0.1 mg/kg, about 0.1 mg/kg to about 0.5 mg/kg, about 0.5 mg/kg to about 1.5 mg/kg, about 1.5 mg/kg to about 2.5 mg/kg, or about 2.5 mg/kg to about 10 mg/kg. In some embodiments, the anti-IL-31RA antibody is administered at a dose of about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg. In some embodiments, the anti-IL-31RA antibody is administered between 30 mg and 60 mg. In some embodiments, the anti-IL-31RA antibody is administered at 30 mg. In some embodiments, the anti-IL-31RA antibody is administered at 60 mg.

In some embodiments, the anti-IL-31RA antibody comprises a heavy chain variable region comprising a HCDR1 comprising SEQ ID NO: 8, a HCDR2 comprising SEQ ID NO: 9, and a HCDR3 comprising SEQ ID NO: 10, and a light chain variable region comprising a LCDR1 comprising SEQ ID NO: 12, a LCDR2 comprising SEQ ID NO: 13, and a LCDR3 comprising SEQ ID NO: 14. In some embodiments, the anti-IL-31RA antibody is nemolizumab or a fragment or variant thereof. In some embodiments, the anti-IL-31RA antibody is nemolizumab.

In some embodiments, the anti-IL-31RA antibody (e.g., nemolizumab) is administered according to a loading dose regimen. In some embodiments, the anti-IL-31RA antibody (e.g., nemolizumab) is administered subcutaneously at a loading dose of 60 mg, followed by a dose of 30 mg every four weeks for at least 8, 12, 14, 16, 18, 20, 22, or 24 weeks.

In some embodiments, the anti-IL-31RA antibody (e.g., nemolizumab) is administered according to a flat dosing regimen. For example, in some embodiments, the anti-IL-31RA antibody (e.g., nemolizumab) is administered subcutaneously at a dose of 30 mg or 60 mg every four weeks for at least 8, 12, 14, 16, 18, 20, 22, or 24 weeks.

In another aspect, the present disclosure provides a method of determining whether a subject with atopic dermatitis (AD) will respond to treatment with an anti-IL-31RA antibody, comprising detecting in a sample obtained from a subject suspected of having AD the expression level of at least one, at least two, at least three, at least four, at least five, or all six biomarker(s) selected from CCL20, CCL22, CCL27, VEGF, IL1RA, and CCL18, and comparing the expression level of the biomarkers to a reference level, wherein the subject will respond to treatment if the expression level of the biomarkers is higher than the reference level, and wherein the reference level is the corresponding level of expression for each biomarker in a skin sample from an individual that does not have AD or a sample of non-lesional skin from the subject. In some embodiments, the sample obtained from the subject suspected of having AD is a skin sample, which optionally comprises a lesion. For the purposes of the foregoing aspect, a skin sample may comprise, consist of, or consist essentially of stratum corneum.

In yet another aspect, the present disclosure provides a method of determining whether a subject with atopic dermatitis (AD) is responding to treatment with an anti-IL-31RA antibody, comprising detecting in a post-treatment sample obtained from a subject with AD that has been administered at least one dose of an anti-IL-31RA antibody the expression level of at least one, at least two, at least three, at least four, at least five, or all six biomarker(s) selected from CCL20, CCL22, CCL27, VEGF, IL1RA, and CCL18, and comparing the expression level of the biomarkers to a baseline level of expression from a sample obtained from the same subject before treatment was commenced, wherein a decrease in the expression level of CCL20, CCL22, CCL27, VEGF, and/or CCL18 is indicative of the subject responding to treatment and/or an increase in the expression level of IL1RA is indicative of the subject responding to treatment. In some embodiments, the sample is a skin sample, which optionally comprises a lesion. In some embodiments, the post-treatment sample is obtained about 4 weeks, about 8 weeks, about 12 weeks, about 14 week, about 16 weeks, about 18 weeks, or about 20 weeks after administration of the anti-IL-31RA antibody. In some embodiments, the anti-IL-31RA antibody comprises a heavy chain variable region comprising a HCDR1 comprising SEQ ID NO: 8, a HCDR2 comprising SEQ ID NO: 9, and a HCDR3 comprising SEQ ID NO: 10, and a light chain variable region comprising a LCDR1 comprising SEQ ID NO: 12, a LCDR2 comprising SEQ ID NO: 13, and a LCDR3 comprising SEQ ID NO: 14. In some embodiments, the anti-IL-31RA antibody is nemolizumab or a fragment or variant thereof. In some embodiments, the anti-IL-31RA antibody is nemolizumab. For the purposes of the foregoing aspect, a skin sample may comprise, consist of, or consist essentially of stratum corneum.

For the purposes of the disclosed treatments and preventions, treatment with an anti-IL-31RA antibody (e.g., nemolizumab) may decrease expression of CCL18 in the lesional skin of a subject with AD (or suspected of having AD) by about 6-fold, about 6.5-fold, about 7-fold, about 7.5-fold, about 8-fold, about 8.5 fold or more. Treatment duration necessary to achieve such a decrease may be 4 weeks, 6 weeks, 8 weeks, 10 weeks, 12 weeks, 14 weeks, 16 weeks, 18 weeks, 20 weeks, or more. Applicable dosing and administration regimens are discussed in more detail below.

For the purposes of the disclosed treatments and preventions, treatment with an anti-IL-31RA antibody (e.g., nemolizumab) may decrease expression of CCL20 in the lesional skin of a subject with AD (or suspected of having AD) by about 2-fold, about 2.25-fold, about 2.5-fold, about 2.75-fold, about 3-fold, about 3.25-fold, about 3.5-fold or more. Treatment duration necessary to achieve such a decrease may be 4 weeks, 6 weeks, 8 weeks, 10 weeks, 12 weeks, 14 weeks, 16 weeks, 18 weeks, 20 weeks, or more. Applicable dosing and administration regimens are discussed in more detail below.

For the purposes of the disclosed treatments and preventions, treatment with an anti-IL-31RA antibody (e.g., nemolizumab) may decrease expression of CCL22 in the lesional skin of a subject with AD (or suspected of having AD) by about 1.1, about 1.2, about 1.3, about 1.4, about 1.5, about 1.6, about 1.7, about 1.8, about 1.9, about 2-fold, about 2.25-fold, about 2.5-fold, about 2.75-fold, about 3-fold, about 3.25-fold, about 3.5-fold or more. Treatment duration necessary to achieve such a decrease may be 4 weeks, 6 weeks, 8 weeks, 10 weeks, 12 weeks, 14 weeks, 16 weeks, 18 weeks, 20 weeks, or more. Applicable dosing and administration regimens are discussed in more detail below.

For the purposes of the disclosed treatments and preventions, treatment with an anti-IL-31RA antibody (e.g., nemolizumab) may decrease expression of CCL27 in the lesional skin of a subject with AD (or suspected of having AD) by about 1.5-fold, about 1.75-fold, about 2-fold, about 2.25-fold, about 2.5-fold, about 2.75 fold, about 3-fold, about 3.25-fold, about 3.5-fold or more. Treatment duration necessary to achieve such a decrease may be 4 weeks, 6 weeks, 8 weeks, 10 weeks, 12 weeks, 14 weeks, 16 weeks, 18 weeks, 20 weeks, or more. Applicable dosing and administration regimens are discussed in more detail below.

For the purposes of the disclosed treatments and preventions, treatment with an anti-IL-31RA antibody (e.g., nemolizumab) may decrease expression of VEGF in the lesional skin of a subject with AD (or suspected of having AD) by about 1.1, about 1.2, about 1.3, about 1.4, about 1.5, about 1.6, about 1.7, about 1.8, about 1.9, about 2-fold, about 2.25-fold, about 2.5-fold, about 2.75-fold, about 3-fold, about 3.25-fold, about 3.5-fold or more. Treatment duration necessary to achieve such a decrease may be 4 weeks, 6 weeks, 8 weeks, 10 weeks, 12 weeks, 14 weeks, 16 weeks, 18 weeks, 20 weeks, or more. Applicable dosing and administration regimens are discussed in more detail below.

For the purposes of the disclosed treatments and preventions, treatment with an anti-IL-31RA antibody (e.g., nemolizumab) may increase expression of IL1RA in the lesional skin of a subject with AD (or suspected of having AD) by about 1.1, about 1.2, about 1.3, about 1.4, about 1.5, about 1.6, about 1.7, about 1.8, about 1.9, about 2-fold, about 2.25-fold, about 2.5-fold, about 2.75-fold, about 3-fold, about 3.25-fold, about 3.5-fold or more. Treatment duration necessary to achieve such a decrease may be 4 weeks, 6 weeks, 8 weeks, 10 weeks, 12 weeks, 14 weeks, 16 weeks, 18 weeks, 20 weeks, or more. Applicable dosing and administration regimens are discussed in more detail below.

For the purposes of the disclosed treatments and preventions, the other noted biomarkers (e.g., CXCL6, CXCL8, Cystatin, FASL, Galectin, IL11, IL21, SPD, and CCL17) may show similar patterns of overexpression in the lesional skin of a subject with AD (or suspected of having AD) compared to expression levels in a reference sample (e.g., non-lesional skin of the subject or skin from a subject without AD), and CX3CL1 may show decreased expression in the lesional skin of a subject with AD (or suspected of having AD) compared to expression levels in a reference sample (e.g., non-lesional skin of the subject or skin from a subject without AD). Similarly, CXCL6, CXCL8, Cystatin, FASL, Galectin, CCL17, and IL21 may show similar patterns of decreases of expression in the lesional skin of a subject with AD (or suspected of having AD) after treatment with an anti-IL-31RA antibody (e.g., nemolizumab), and CX3CL1, IL11, and SPD may show increases in expression in the lesional skin of a subject with AD (or suspected of having AD) after treatment with an anti-IL-31RA antibody (e.g., nemolizumab).

In any of the preceding aspects or embodiments, the expression level of these genes (e.g., CCL20, CCL22, CCL27, VEGF, IL1RA, and/or CCL18) in the skin lesion may be detected using any nucleic acid or polypeptide detection assay known in the art. In some embodiments, expression of one or more of CCL20, CCL22, CCL27, VEGF, IL1RA, and/or CCL18 is determined by RT-qPCR, RT-PCR, RNA-seq, Northern blotting, Serial Analysis of Gene Expression (SAGE), DNA or RNA microarrays, Western blotting, ELISA, surface plasmon resonance, or mass spectrometry.

### VII. Doses and Dosing Regimen for the Disclosed Methods and Uses

An effective amount of an anti-IL-31RA antibody, such as nemolizumab or a fragment or variant thereof, is an amount sufficient to effect beneficial or desired results such as alleviating at least one or more symptom of AD. An effective amount as used herein would also include an amount sufficient to delay or prevent the development, alter the course of an AD symptom, or reverse a symptom of AD. Thus, it is not possible to specify the exact “effective amount.” However, for any given case, an appropriate “effective amount” can be determined by one of ordinary skill in the art using only routine experimentation.

An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present disclosure for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment and prevention dosages generally may be titrated to optimize safety and efficacy. The dosage can be determined by a physician and adjusted, as necessary, to suit observed effects of the treatment. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks.

Dosage regimens for treating or preventing AD may comprise flat dosing (i.e., administering the same dose repeatedly at pre-determined intervals) or comprise a loading dose (i.e., administrating an initial dose that is higher or different than subsequent, serial doses). For the purposes of either type of dosing regimen an effective dose may be administered topically, parenterally, subcutaneously, subdermally, intradermally, or intramuscularly. In preferred embodiments, administration comprises subcutaneous injection.

In some embodiments, a loading dose and the subsequent serial doses may be administered via the same route (e.g., subcutaneously), while in some embodiments, a loading dose and the subsequent serial doses may be administered via different routes (e.g., parenterally and subcutaneously, respectively). In some embodiments, the loading dose may be about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, or higher. In some embodiments, the loading dose may be 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, or higher. In some embodiments, the loading dose may be about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3 mg/kg, about 3.5 mg/kg, about 4 mg/kg, about 4.5 mg/kg, about 5 mg/kg, about 6 mg/kg, about 7 mg/kg, about 8 mg/kg, about 9 mg/kg, about 10 mg/kg, about 15 mg/kg, about 25 mg/kg, about 50 mg/kg, about 75 mg/kg, about 100 mg/kg, about 500 mg/kg, or about 1,000 mg/kg. In some embodiments, the loading dose may be 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3 mg/kg, 3.5 mg/kg, 4 mg/kg, 4.5 mg/kg, 5 mg/kg, 6 mg/kg, 7 mg/kg, 8 mg/kg, 9 mg/kg, 10 mg/kg, 15 mg/kg, 25 mg/kg, 50 mg/kg, 75 mg/kg, 100 mg/kg, 500 mg/kg, or 1,000 mg/kg. In some embodiments, the loading dose is administered as a single injection. In some embodiments, the loading dose is administered as multiple injections, which may be administered at the same time or spaced apart at defined intervals.

The subsequent serial doses of a loading dose regimen are generally lower than the loading dose. For examples, in some embodiments, the dosing regimen may comprise a loading dose of 60 mg and a serial dose of 30 mg, which may be administered a defined interval of, for example, every 4 weeks. In some embodiments, the serial dose of a dosing regimen may be about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, or higher. In some embodiments, the serial dose may be 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, or higher. In some embodiments, the serial dose may be about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3 mg/kg, about 3.5 mg/kg, about 4 mg/kg, about 4.5 mg/kg, about 5 mg/kg, about 6 mg/kg, about 7 mg/kg, about 8 mg/kg, about 9 mg/kg, about 10 mg/kg, about 15 mg/kg, about 25 mg/kg, about 50 mg/kg, about 75 mg/kg, about 100 mg/kg, about 500 mg/kg, or about 1,000 mg/kg. In some embodiments, the serial dose may be 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3 mg/kg, 3.5 mg/kg, 4 mg/kg, 4.5 mg/kg, 5 mg/kg, 6 mg/kg, 7 mg/kg, 8 mg/kg, 9 mg/kg, 10 mg/kg, 15 mg/kg, 25 mg/kg, 50 mg/kg, 75 mg/kg, 100 mg/kg, 500 mg/kg, or 1,000 mg/kg.

For the purposes of a loading dose regimen, the first serial dose may be administered 1 day, 2 days, 3 days, 4 days, 5 days, 6 days, 7 days, 8 days, 9 days, 10 days, 11 days, 12 days, 13 days, 1 week, 2 weeks, 3 weeks, 4 weeks, 5 weeks, 6 weeks, 7 weeks, 8 weeks, 9 weeks, or 10 weeks after the initial loading dose. In some embodiments, the first serial dose is administered 4 weeks after the initial loading dose. In some embodiments, the subsequent serial doses are administered once every 1 day, 2 days, 3 days, 4 days, 5 days, 6 days, 7 days, 8 days, 9 days, 10 days, 11 days, 12 days, 13 days, 1 week, 2 weeks, 3 weeks, 4 weeks, 5 weeks, 6 weeks, 7 weeks, 8 weeks, 9 weeks, or 10 weeks. In some embodiments, the serial doses are spaced 4 weeks apart (i.e., nemolizumab or a fragment or variant thereof is administered once every 4 weeks).

In some embodiments, the dose of an anti-IL31RA antibody (e.g., nemolizumab or a fragment or variant thereof) administered to the subject can be within the range of 0.001 to 1,000 mg/kg body weight of the subject. In some embodiments, the dose ranges from about 0.01 to 50 mg/kg, about 0.01 mg/kg to about 0.1 mg/kg, about 0.05 mg/kg to 0.15 mg/kg, about 0.1 mg/kg to about 0.6 mg/kg, about 0.1 mg/kg to about 1 mg/kg, about 0.25 mg/kg to about 0.75 mg/kg, about 0.4 mg/kg to about 0.8 mg/kg, about 0.4 mg/kg to about 1.8 mg/kg, about 0.5 to about 2.5 mg/kg, about 0.8 mg/kg to about 2.2 mg/kg, about 1 mg/kg to about 2.5 mg/kg, about 1 mg/kg to about 3.5 mg/kg, about 1 mg/kg to about 5 mg/kg, about 2 mg/kg to about 4 mg/kg, about 2.5 mg/kg to about 10 mg/kg, about 5 mg/kg to about 10 mg/kg, about 10 mg/kg to about 20 mg/kg, about 10 mg/kg to about 40 mg/kg, about 20 mg/kg to about 50 mg/kg, about 25 mg/kg to about 75 mg/kg, about 50 mg/kg to about 100 mg/kg, or about 100 mg/kg to about 500 mg/kg, or about 100 mg/kg to about 1000 mg/kg body weight of an anti-IL31RA antibody (e.g., nemolizumab or a fragment or variant thereof). In preferred embodiments, the dose ranges from about 0.01 mg/kg to about 0.1 mg/kg, about 0.1 mg/kg to about 0.5 mg/kg, about 0.5 mg/kg to about 1.5 mg/kg, about 1.5 mg/kg to about 2.5 mg/kg, or about 2.5 mg/kg to about 10 mg/kg. In some embodiments, the dose is about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3 mg/kg, about 3.5 mg/kg, about 4 mg/kg, about 4.5 mg/kg, about 5 mg/kg, about 6 mg/kg, about 7 mg/kg, about 8 mg/kg, about 9 mg/kg, about 10 mg/kg, about 15 mg/kg, about 25 mg/kg, about 50 mg/kg, about 75 mg/kg, about 100 mg/kg, about 500 mg/kg, or about 1,000 mg/kg. In particular embodiments, the dose of an anti-IL31RA antibody (e.g., nemolizumab or a fragment or variant thereof) is about 0.1 mg/kg, about 0.5 mg/kg, about 1 mg/kg, about 1.5 mg/kg, about 2 mg/kg, or about 2.5 mg/kg. In a preferred embodiment, the dose is about 0.5 mg/kg.

In some embodiments, the dose of an anti-IL31RA antibody (e.g., nemolizumab or a fragment or variant thereof) administered to the subject is within the range of 1 to 100 mg, 25 to 75 mg, 30 to 60 mg, 40 to 80 mg, 20 to 80 mg, 1 to 25 mg, 1 to 50 mg, 10 to 90 mg, 15 to 85 mg, or ranges there between. In some embodiments, the dose may be about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, or higher. In some embodiments, the dose may be 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, or higher.

In some embodiments of the disclosed methods and uses, a loading dose of about 60 mg of an anti-IL31RA antibody (e.g., nemolizumab or a fragment or variant thereof) may be administered to a subject with AD, followed by subsequent serial doses of the anti-IL31RA antibody (e.g., nemolizumab of a fragment or variant thereof) at about 30 mg once every 4 weeks. In some embodiments of the disclosed methods and uses a first dose of about 60 mg of an anti-IL31RA antibody (e.g., nemolizumab or a fragment or variant thereof) may be administered to a subject with AD, followed by subsequent serial doses of the anti-IL31RA antibody (e.g., nemolizumab of a fragment or variant thereof) at about 60 mg once every 4 weeks (i.e., the dose remains constant or is a “flat” dosing regimen). In some embodiments of the disclosed methods and uses a first dose of about 30 mg of an anti-IL31RA antibody (e.g., nemolizumab or a fragment or variant thereof) may be administered to a subject with AD, followed by subsequent serial doses of the anti-IL31RA antibody (e.g., nemolizumab of a fragment or variant thereof) at about 30 mg once every 4 weeks.

In some embodiments of the disclosed methods and uses, an anti-IL31RA antibody (e.g., nemolizumab or a fragment or variant thereof) is administered by a topical or parenteral route. In some embodiments, an anti-TL31RA antibody (e.g., nemolizumab or a fragment or variant thereof) is administered subcutaneously. In some embodiments, the dose is administered subcutaneously at or proximal to a site of one or more nodules, lesions, or excoriations.

In some embodiments of the disclosed methods and uses, an anti-IL31RA antibody (e.g., nemolizumab or a fragment or variant thereof) is administered daily, every other day, twice per week, three times per week, four times per week, five times per week, six times per week, once per week, once every two weeks, once every three weeks, once every four weeks, once every five weeks, once every six weeks, once every seven weeks, once every eight weeks, once every nine weeks, once every 10 weeks, once every 11 weeks, once every 12 weeks, twice per year, once per year, and/or as needed based on the appearance of symptoms of AD. In preferred embodiments, an anti-IL31RA antibody (e.g., nemolizumab or a fragment or variant thereof) is administered every four weeks or every eight weeks.

In some embodiments of the disclosed methods and uses, the duration of treatment or prevention is about one day, about one week, about two weeks, about three weeks, about four weeks, about five weeks, about six weeks, about seven weeks, about eight weeks, about nine weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 24 weeks, about 30 weeks, about 36 weeks, about 40 weeks, about 48 weeks, about 50 weeks, about one year, about two years, about three years, about four years, about five years, or as needed based on the appearance of symptoms of AD. In preferred embodiments, duration of treatment or prevention is about 12 weeks to about 24 weeks, about 12 to about 36 weeks, about 12 to about 48 weeks, or about 24 to about 36 weeks.

The present disclosure provides uses of an anti-IL31RA antibody (e.g., nemolizumab or a fragment or variant thereof) in the manufacture of a medicament for the treatment or prevention of AD, for normalizing one of more of CCL20, CCL22, CCL27, VEGF, IL1RA, and CCL18 expression in subjects with AD, and/or for decreasing inflammatory responses in the skin. All of the disclosed doses, dosing regimens, routes of administrations, biomarkers, and therapeutic endpoints are applicable to these uses as well.

The following examples are given to illustrate the present disclosure. It should be understood that the invention is not to be limited to the specific conditions or details described in these examples.

### EXAMPLES

### Example 1—Clinical Trial for Determine Efficacy of Nemolizumab in the Treatment of AD

Introduction and Methods

A Phase 2, single-arm, open-label study was conducted as described below to evaluate the pharmacokinetics (PK), safety and clinical efficacy of nemolizumab in 18 subjects with moderate-to-severe AD. Effect of nemolizumab on biomarkers of AD was also investigated.

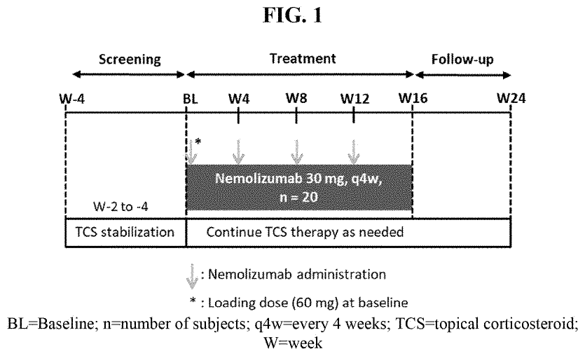

Overall study design. This was an open-label, single-group study to evaluate the PK and safety of nemolizumab in adolescent subjects (12 to 17 years of age), with moderate-to-severe AD and associated pruritus. Approximately 20 eligible subjects were enrolled to receive subcutaneous injections of nemolizumab (30 mg) every 4 weeks over a 16-week treatment period, with an LD of 60 mg on Day 1. The final dose of study drug occurred at Week 12 and subjects completed the treatment period at the Week 16 visit. An 8-week follow-up period was scheduled (Week 24 visit) for subjects who did not rollover into the nemolizumab long-term extension study. Subjects who prematurely discontinued the study before the Week 16 visit had to be followed for 12 weeks after their last dose of study drug. FIG. 1 and Table 2 display the clinical study schematic.

TABLE 2
Clinical study schematic
Screening
↓
Enrolment (~20 subjects)

| | |
|---|---|
| | Group 1 (n = ~20) |
| Treatment | nemolizumab 30 mg |
| | (LD at the baseline visit: 60 mg) |
| Treatment Frequency | Q4W |
| Treatment Duration | 16 weeks (last study drug |
| | injection at Week 12) |
| Duration of | 8 weeks (from Week 16 |
| Follow-up Period | to Week 24) |

LD = loading dose;
n = number of subjects;
Q4W = every 4 weeks.

Nemolizumab serum concentration was assessed at baseline, Weeks 1-2, 4, 8, 12, 16, and Week 24 and at any unscheduled visit for safety reasons. Previous PK data from the Phase 1 and Phase 2a studies were used to develop a population PK model that allows an accurate simulation of nemolizumab serum concentrations with different doses and dose regimens. The population PK model was used to simulate systemic exposure using several fixed doses for the Phase 2b study. The Phase 2b results confirm the predictability of the popPK model for the 30 mg dose in adults (Table 3).

TABLE 3
Nemolizumab predicted average serum concentrations (μg/mL)
compared to actual C_{trough} level for the 30 mg dose in adults

| C_{trough} (μg/mL) | Predicted serum | Actual serum |
|---|---|---|
| Mean ± SD | concentrations^{a} | concentration^{b} |
| Week 4 | 2.62 ± 0.884 | 2.67 ± 0.946 |
| | (N = 110) | (n = 54) |
| Week 8 | 2.26 ± 0.882 | 2.30 ± 0.986 |
| | (N = 109) | (n = 51) |
| Week 12 | 2.19 ± 0.828 | 2.24 ± 0.915 |
| | (N = 89) | (n = 36) |
| Week 16 | 2.15 ± 0.845 | 2.29 ± 0.963 |
| | (N = 79) | (n = 27) |

^{a}N represents the number of observations. Concentrations are the individual predictions derived from the model.
^{b}n represents the number of adult subjects that provide PK data for the interim PK analysis of study 114322.

Objectives:

The primary objective of the study was to assess the PK and safety of nemolizumab in adolescent subjects with moderate-to-severe atopic AD and associated pruritus not adequately controlled with topical treatments, when administered concomitantly with TCS.

The secondary objective of the study was to evaluate the efficacy of nemolizumab and to further characterize the relationship between nemolizumab systemic exposure and clinical efficacy endpoints (PK/PD relationship).

Inclusion criteria. In order to participate in the study, subjects must have met the following criteria:

1. Subjects≥12 to 17 years of age at the screening visit.
2. Chronic AD for at least 2 years before the screening visit and confirmed according to the American Academy of Dermatology Consensus Criteria at the time of the screening visit.
3. EASI score≥16 at both screening and baseline visits.
4. IGA score≥3 (based on the IGA scale ranging from 0 to 4, in which 3 was moderate and 4 was severe) at both screening and baseline visits.
5. AD involvement≥10% of BSA at both screening and baseline visits.
6. Peak (maximum) pruritus numeric rating scale (NRS) score of at least 4.0 at both screening and baseline visits
Screening PP NRS score was determined by a single PP NRS assessment (score ranging from 0 to 10) for the 24-hour period immediately preceding the screening visit.
Baseline PP NRS score was determined based on the average of daily PP NRS scores (score ranging from 0 to 10) during the 7 days immediately preceding baseline (rounding not permitted). A minimum of 4 daily scores out of the 7 days immediately preceding baseline was required for this calculation.
   7. Documented recent history (within 6 months before the screening visit) of inadequate response to topical medications (TCS with or without TCI). Acceptable documentation includes patient records with information on TCS (with or without TCI) prescription and treatment outcome, or written documentation of the conversation with the subject's treating physician, if different than the investigator. If documentation was inadequate, subjects may have been rescreened after such documentation was obtained. Inadequate response to TCS treatments (with or without TCI) was defined as:

7a. Failure to achieve or maintain remission or low disease activity (equivalent to IGA≤2, mild) despite treatment with a daily regimen of a medium or high potency TCS (with or without TCI), applied for at least 4 weeks or for the maximum duration per prescribing information; or

7b. Requirement of a long-term treatment (>4 weeks) with a high potency TCS (with or without TCI) to achieve or maintain remission or low disease activity (equivalent to IGA≤2). Note: If subjects had received a TCI in addition to a TCS, documentation on failure to achieve or maintain remission or low disease activity (equivalent to IGA≤2) was also required.

7c. If documentation of inadequate response to topical medications was not available, subjects with a documented recent course of systemic treatment or phototherapy for AD (within 6 months before the visit) were also considered as inadequate responders to topical treatments.

8. Agreed to apply a moisturizer throughout the study from the screening visit daily, and liberally as needed; agreed to apply an authorized TCS from the screening visit and throughout the study as determined appropriate by the investigator.
9. Women of childbearing potential (WOCBP) must have agreed either to be strictly abstinent throughout the study and for 12 weeks after the last study drug injection or to use an effective and approved method of contraception throughout the study and for 12 weeks after the last study drug injection. This criterion also applied to a prepubertal female subject who began menses during the study.

Effective and approved methods of contraception applicable for the subject and/or their partner are defined below:
Progestogen-only oral hormonal contraception
Male or female condom
Cap, diaphragm, or sponge with spermicide
Combination of male or female condom with cap, diaphragm, or sponge with spermicide
Combined (estrogen and progestogen containing) oral, intravaginal, or transdermal hormonal contraception
Injectable or implanted hormonal contraception
Intrauterine devices
   10. Subject and guardian willing and able to comply with all of the time commitments and procedural requirements were of the clinical study protocol, including daily diary recordings by the subject using an electronic handheld device provided for this study.
   11. Understood and signed an ICF and Assent Form before any investigational procedures were performed.

Exclusion criteria. In order to participate in the study, subjects must not have met any of the following criteria:

1. Body weight<30 kg.
2. Subjects meeting 1 or more of the following criteria at screening or baseline:

2a. Had an asthma exacerbation requiring hospitalization in the preceding 12 months.

2b. Reporting asthma that has not been well-controlled (i.e., symptoms>2 days per week, nighttime awakenings >1 to 3 times per week, or some interference with normal activities) during the preceding 3 months.

2c. Asthma control test (ACT)≤19 (applies only for subjects with a history of asthma).

2d. Peak expiratory flow (PEF)<80% of the predicted value.

3. Subjects with a current medical history of chronic obstructive pulmonary disease (COPD) and/or chronic bronchitis.
4. Cutaneous infection within 1 week before the screening visit or any infection requiring treatment with oral or parenteral antibiotics, antivirals, antiparasitics, or antifungals within 1 week before the screening visit. Subjects may have been rescreened once the infection had resolved.
5. Requiring rescue therapy for AD during the run-in period or expected to require rescue therapy within 2 weeks following the baseline visit.
6. Positive serology results for hepatitis B surface antigen [HBsAg] or hepatitis B core antibody [HBcAb], hepatitis C antibody, or human immunodeficiency virus [HIV] antibody at the screening visit. Note: Subjects with a positive HBcAb and a negative HBsAg could have been included in this clinical study if hepatitis B surface antibody (HBsAb) was positive (considered immune after a natural infection).
7. Received any of the following treatments in Table 4 within the specified timeframe before the baseline visit:

TABLE 4
Prior treatments

| Treatment(s): | Timeframe |
|---|---|
| Non-live vaccine | 2 weeks |
| Coal tar products | 2 weeks |
| Topical phosphodiesterase (PDE)-4 inhibitor | 2 weeks |
| Topical calcineurin inhibitor | 2 weeks |
| Non-authorized TCS | 2 weeks |
| Topical medications, including authorized TCS, with occlusive | 2 weeks |
| dressings (e.g., wet wraps) | |
| Systemic corticosteroids (corticosteroid inhalers were permitted) | 4 weeks |
| Phototherapy or tanning beds | 4 weeks |
| Immunosuppressive or immunomodulatory drugs (e.g, | 4 weeks or 5 half-lives |
| cyclosporine A, oral tacrolimus, cyclophosphamide, azathioprine, | (whichever was longer) |
| methotrexate, mycophenolate mofetil) | |
| Biologies and their biosimilars (e.g, dupilumab, etanercept, | 8 weeks or 5 half-lives |
| adalimumab, infliximab, omalizumab, etc) | (whichever was longer) |
| Live or attenuated vaccine | 12 weeks |
| Investigational topical or systemic medication (e.g, topical or oral | 16 weeks |
| Janus kinase [JAK] inhibitors) | |
| Drugs with a sedative effect such as benzodiazepines, | 1 week |
| imidazopyridines, barbiturates, or sedative antidepressants (e.g, | |
| amitryptiline) | |
| Note: Stable treatment with antihistamines with or without | |
| sedative effect was permitted. | |
| Gabapentinoids (e.g, gabapentin, pregabalin) | 4 weeks |
| Alternative medicine for AD (e.g, traditional Chinese medicine) | 2 weeks |

AD = atopic dermatitis;
JAK = janus kinase;
TCS = topical corticosteroid
Note:
These treatments should not have been discontinued for reasons related to this clinical study.

Treatments. Nemolizumab is a humanized monoclonal modified immunoglobulin G (IgG) 2 antibody comprising a structure of 2 H-chains (445 amino acid residues) and 2 L-chains (214 amino acid residues) connected by 16 disulfide bonds. The drug product used in this study was a vial containing 153 mg lyophilized nemolizumab powder that was stored protected from light at 2 to 8° C. until reconstitution with 1.3 mL of sterile water and subsequent dilution using nemolizumab placebo, resulting in an injectable solution containing 100 g/mL of nemolizumab that was further diluted to yield the appropriate dosing solution. Each 100 mg/mL vial was subsequently diluted with reconstituted placebo in order to obtain 30 mg/mL or 60 mg/mL (LD). By design, the volume of 1 mL was consistently to be injected. The reconstitution with sterile water for injection and dilution with nemolizumab placebo was identical to steps used in the adult Phase 2b study (SPR.114322). The dosing scheme and detailed descriptions of the drug are provided in Table 5 and Table 6.

TABLE 5
Dosing scheme

| Day 1 | Weeks 4, 8, and 12 |
|---|---|
| 60 mg | 30 mg |

TABLE 6
Description of nemolizumab

| Name | Nemolizumab |
|---|---|
| Internal code | CD14152 |
| Pharmaceutical form | Lyophilized powder |
| Packaging | Vial |
| Dosage | 30 mg (with a loading dose |
| | of 60 mg at baseline) |
| Route | Subcutaneous injection (1 mL) |
| Dose regimen | Every 4 weeks (Q4W) |
| Treatment duration | 16 weeks (last injection at Week 12) |

The prescribed use of background therapies was documented in the case report form (CRF). Background therapies were required throughout the study (screening through the follow-up visit), as described below.

Moisturizer: subjects were required to apply a moisturizer daily, and liberally as needed, to dry skin and AD lesions throughout the study. The subject's current moisturizer or a moisturizer recommended by the investigator may have been used. Use should have not occurred within 8 hours before each clinic visit. Moisturizer was not considered a topical AD medication.

Topical Corticosteroid therapy (TCS): subjects were required to apply the authorized background TCS therapy to all AD lesions beginning within the screening period and >14 days before Day 1, and throughout the study as directed by the investigator. Subjects were required to apply a medium potency TCS in areas of the body where use of medium potency TCS was considered safe (e.g., trunk and extremities). A low potency TCS was used on TCS-sensitive areas (e.g., face, neck, intertriginous areas). Subjects were required to apply a thin layer of authorized TCS on all AD lesions at a frequency that was necessary to ensure disease stability and prevent AD flare, but which did not exceed the daily frequency recommended in the product labelling. It should be noted that “as needed” use of TCS was not permitted.

Rescue therapy: if deemed medically necessary by the investigator (e.g., to control intolerable AD signs/symptoms), rescue therapies were prescribed to the subjects at any time during the study except during the run-in period. Subjects who received rescue therapies during the run-in period were not eligible to participate in the study. As a general guideline and per individual investigator judgment, rescue therapy was not prescribed within the first 2 weeks after baseline (i.e., Week 2 visit) to allow a minimum period for study drug exposure in the presence of background therapy. Rescue treatments were only treatments that directly treated AD (mainly those that were approved or were standard of care) and included topical and systemic treatments as outlined. Some rescue therapies included: TCI; higher potency of TCS (class I-II according to the US classification); oral corticosteroids; biologics (including their biosimilars); systemic nonsteroidal immunosuppressants/immunomodulators; and phototherapy.

Efficacy, Safety, and Pharmacokinetic Variables

Assessments of PK, safety, and efficacy were conducted throughout the study. Subject-reported assessments of pruritus, sleep disturbance and the use of topical AD medication for their eczema were collected daily. The schedule of assessments is provided in Table 7.

TABLE 7
Schedule of assessments

| | Screening | Treatment Period | | | | | | Follow-up | | |
|---|---|---|---|---|---|---|---|---|---|---|
| | Visit | | | | | | | | | |
| | Visit 1/ | Visit 2^{a}/ | | | | | | Visit 8^{a, d}/ | | |
| | Run-in | Baseline | Visit 3^{b} | Visit 4^{a} | Visit 5^{a} | Visit 6^{a} | Visit 7^{a, c} | Final | | |
| | Week | | | | | | | | | |
| | −4 to −2 | (1) | 1 to 2 | 4 | 8 | 12 | 16^{c} | 24 | Unscheduled | Early |
| | Day(s) | | | | | | | | Visit^{a, e} | Termination |
| Assessment(s) | −28 to −15 | 1 | 10 (±3) | 29 (±3) | 57 (±3) | 85 (±5) | 113 (±5) | 169 (±5) | (if applicable) | Visit^{a, f} |
| Informed consent | X | | | | | | | | | |
| and assent form | | | | | | | | | | |
| Inclusion/exclusion | X | X | | | | | | | | |
| criteria | | | | | | | | | | |
| Demographic data | X | | | | | | | | | |
| Medical history, | X | X | | | | | | | | |
| previous therapy | | | | | | | | | | |

EFFICACY/PATIENT-REPORTED OUTCOME ASSESSMENTS

| Peak pruritus NRS^{h} | X------------------------------------------------------------------------------------------------------------------------ X | | | | | | | | | |
|---|---|---|---|---|---|---|---|---|---|---|
| (daily) | | | | | | | | | | |
| Average pruritus | X------------------------------------------------------------------------------------------------------------------------ X | | | | | | | | | |
| NRS^{h} (daily) | | | | | | | | | | |
| Sleep disturbance | X------------------------------------------------------------------------------------------------------------------------ X | | | | | | | | | |
| NRSⁱ (daily) | | | | | | | | | | |
| Topical AD | X------------------------------------------------------------------------------------------------------------------------ X | | | | | | | | | |
| medication used | | | | | | | | | | |
| (daily)^{j} | | | | | | | | | | |
| ACT^{g, m} | X^{k} | X^{k} | X^{k} | X^{k} | X^{k} | X^{k} | X^{k} | X^{k} | (X)^{k} | X^{k} |
| DLQI/cDLQI^{g, l} | | X | | | | | X | | | X |
| EASI | X | X | X | X | X | X | X | X | (X) | X |
| IGA | X | X | X | X | X | X | X | X | (X) | X |
| BSA | X | X | X | X | X | X | X | X | (X) | X |
| SCORAD | X | X | X | X | X | X | X | X | (X) | X |

SAFETY/LABORATORY ASSESSMENTS

| | | | | | | | | | | |
|---|---|---|---|---|---|---|---|---|---|---|
| Physical | X | X | X | X | X | X | X | X | (X) | X |
| examination | | | | | | | | | | |
| (including | | | | | | | | | | |
| respiratory | | | | | | | | | | |
| examination)^{m} | | | | | | | | | | |
| PEF Testing^{m} | X | X | X^{m} | X^{m} | X^{m} | X^{m} | X^{m} | X^{m} | (X)^{m} | X^{m} |
| Height | X | | | | X | | X | X | (X) | X |
| Weight | X | X | X | X | X | X | X | X | (X) | X |
| ECGⁿ | X | | | | | | X | | (X) | X |
| Vital signs^{o} | X | X | X | X | X | X | X | X | (X) | X |
| Urinalysis^{p} | X | X^{p} | | X^{p} | X^{p} | X^{p} | X | X | (X) | X |
| Hematology^{p} | X | X^{p} | | X^{p} | X^{p} | X^{p} | X | X | (X) | X |
| Clinical chemistry^{p} | X | X^{p} | | X^{p} | X^{p} | X^{p} | X | X | (X) | X |
| TB test^{q} | X | | | | | | | | (X) | |
| Hepatitis B and C | X | | | | | | | | | |
| test | | | | | | | | | | |
| HIV test | X | | | | | | | | | |
| Pregnancy test^{r} | X^{r} | X | | X | X | X | X | X | (X) | X |
| Contraceptive | X | | | | | | | | | |
| counseling | | | | | | | | | | |
| Adverse event | X | X | X | X | X | X | X | X | (X) | X |
| reporting | | | | | | | | | | |
| Concomitant | X | X | X | X | X | X | X | X | (X) | X |
| therapy/medications^{s} | | | | | | | | | | |

PKP^{p, t, u} ADA^{p, u}, PD/BIOMARKER^{t} ASSESSMENTS

| PK samples^{p, t, u} | X | X | X | X | X | X | X | X | X |
|---|---|---|---|---|---|---|---|---|---|
| ADA samples^{p, u} | X | | X | X | | X | X | X | X |
| Blood sample(s) for | X | | | X | | X | | | X |
| biomarkers^{u} | | | | | | | | | |
| Stratum corneum | | | | | | | | | |
| samples (using D- | | | | | | | | | |
| squames) for | | | | | | | | | |
| biomarkers^{u} | | | | | | | | | |

STUDY DRUG AND BACKGROUND THERAPY

| Study drug | | X^{v, w} | X^{v} | X | X |
|---|---|---|---|---|---|
| injection | | | | | |
| Background | X--------------------------------------------------------------------------------------- ------------------- X | | | | |
| therapy^{x} | | | | | |

ACT = Asthma Control Test;
AD = atopic dermatitis;
ADA = antidrug antibody;
BSA = Body Surface Area;
cDLQI = Children's Dermatology Life Quality Index;
DLQI = Dermatology Life Quality Index;
EASI = Eczema Area and Severity Index;
ECG = Electrocardiogram;
HIV = Human Immunodeficiency Virus;
IGA = Investigator's Global Assessment;
LTE = long-term extension;
NRS = Numeric Rating Scale;
PE = physical examination;
PEF = peak expiratory flow;
PK = Pharmacokinetics;
SCORAD = SCORing;
TB = Tuberculosis;
TCS = topical corticosteroid;
UPT = urine pregnancy test;
WOCBP = women of childbearing potential,
^{a}Subjects were required to fast for at least 8 hours before the visit(s).
^{b}Visit 3 was a single visit occurring between Week 1 and Week 2.
^{c}For subjects that completed the Week 16 visit and rolled over to the nemolizumab LTE study, the Week 16 visit (Visit 7) served as the final study visit.
^{d}A follow-up visit was required for subjects who discontinued after the Week 16 visit and did not rollover to the nemolizumab LTE study. The follow-up visit was to occur 12 weeks after the last study drug injection.
^{e}Assessments conducted at the unscheduled visit(s) depended on the reason for the visit.
^{f}Subjects who discontinued before the Week 16 visit were to attend an early termination visit and a follow-up/final study visit 12 weeks after the last study drug injection.
^{g}Patient-reported efficacy measurements (ACT and DLQI/cDLQI) were to occur before investigator (efficacy and safety) assessments, blood sample collections, and study dmg administration.
^{h}Peak and average pruritus NRS were to be recorded by subjects once daily in the evening, beginning >2 weeks before Day 1.
ⁱSleep disturbance NRS was to be recorded by subjects once daily in the morning, beginning >2 weeks before Day 1.
^{j}Subjects were asked to record the response (yes/no) to the following question each evening, “Did you apply the eczema medication your doctor gave you, to your skin today?”, beginning ≥2 weeks before Day 1.
^{k}Subjects with a history of asthma were to complete the ACT at each study visit before physical examination and other investigator assessments.Subjects with de novo diagnosed asthma were to complete the ACT beginning at the first visit of de novo diagnosis and at all follow-up study visits.
^{l}Subjects aged 17 years or older were to complete the DLQI, while subjects aged 12 to 16 years old were to complete the cDLQI.
^{m}Complete PE were performed at each visit. At all visits, all subjects were to be asked about respiratory changes and a respiratory examination was also to be done during the PE. In addition, PEF measurements were performed for all subjects at screening and baseline. For subjects reporting a medical history of asthma, PEF measurements and ACT was to be performed at all visits during the clinical trial. Subjects with a medical history of asthma were referred to the physician managing their asthma if ACT ≤19, PEF < 80% of the predicted value, and/or unexpected worsening of asthma was observed or reported. For subjects diagnosed with de novo asthma, PEF measurements and ACT was to be performed at all visits starting with the visit in which the diagnosis was confirmed. Subjects without a medical history of asthma were referred to a respiratory specialist if respiratory changes suggestive of asthma were observed or reported. PEF measurements were to be done under the supervision of qualified study personnel and to have occurred at approximately the same time of day for all visits, when possible. Attempts were to be made to withhold asthma medication on study visit days until after PEF testing was complete, to the extent it did not pose an undue risk to the subject, to avoid interference with PEF measurements.
ⁿECGs were to be performed before any scheduled vital sign measurements and blood draws.
^{o}Vital signs included pulse rate, systolic and diastolic blood pressure (after the subject has been sitting for at least 5 minutes), and body temperature.
^{p}At study drug injection visits, laboratory (hematology, clinical chemistry, and urinalysis), PK, and ADA samples were to be collected ≤30 minutes before study drug injection(s).
^{q}TB screening: QuantiFERON-TB Gold test was performed for all subjects.
^{r}Only for WOCBP. Serum pregnancy test was performed at the screening visit and urine pregnancy test (UPT) for all other visits. For prepubertal subjects, pre-menses status was reconfirmed at every visit and, in case of status change, information on contraceptive measures was collected and a UPT performed according to the schedule for WOCBP.
^{s}At the study drug injection visits (baseline, Weeks 4, 8 and 12), PK samples were collected within 30 minutes before study dmg injection (pre-dose samples).
At Weeks 1 to 2, 16, and 24, attempts were to be made to collect PK samples at approximately the same time of the day, to the extent possible.
^{t}See Stratum corneum biomarker analysis below, along with corresponding table
^{u}Subjects received a loading dose of 60 mg on Day 1.
^{v}Subjects were required to remain at the study center for at least 30 minutes post injection, following the first 2 injections, to monitor for possible hypersensitivity reaction.
wBackground therapy (i.e., moisturizer and authorized TCS) beginning ≥2 weeks before Day 1. Moisturizer should NOT have been used for at least 8 hours before each clinical visit. The investigator should have adjusted background TCS use during the study, according to the disease activity and tolerability, including tapering when signs and symptoms improve, discontinued when lesions clear, and restarted if signs and symptoms recur.

Efficacy measurements were conducted by the investigators (or trained designees) and subjects (for subject-reported efficacy measurements) according to Error! Reference source not found. Whenever possible, the same evaluator made the assessment throughout the study.

Eczema area and severity index. Eczema Area and Severity Index is a validated measure commonly used in clinical studies and clinical practice to assess the severity and the extent of AD signs. Eczema Area and Severity Index is a composite score ranging from 0 to 72. The severity of erythema, induration/papulation, excoriation, and lichenification were assessed by the investigator or trained designee on a scale of 0 (absent) to 3 (severe) for each of the 4 body areas: head/neck, trunk, upper limbs, and lower limbs, with half points allowed. In addition, the extent of AD involvement in each of the 4 body areas were assessed as a percentage by body area of head, trunk, upper limbs and lower limbs, and converted to a score of 0 to 6. The EASI score were calculated in the CRF.

Investigator global assessment (IGA). The IGA is a 5-point scale ranging from 0 (clear) to 4 (severe) used by the investigator or trained designee to evaluate the global severity of AD and the clinical response to a treatment. Treatment success is defined 0 (clear) or 1 (almost clear) and a 2-grade change from baseline.

Body surface area (BSA). The BSA involvement of AD was assessed by the investigator or trained designee for each part of the body (the possible highest score for each region is: head and neck [9%], anterior trunk [18%], back [18%], upper limbs [18%], lower limbs [36%], and genitals [1%]), and was reported as a percentage of all major body sections combined, as calculated in the CRF.

SCORing Atopic Dermatitis (SCORAD). SCORAD is a validated measure commonly used in clinical studies and clinical practice to assess the severity and the extent of AD signs and symptoms. The SCORAD score ranges from 0 to 103 and has 3 components: extent (BSA), signs, and symptoms of AD and subject-reported symptoms of pruritus and sleep loss. The investigator or designee assessed the severity of 6 signs of AD (erythema/darkening, edema/papulation, oozing/crusting, excoriation, lichenification/prurigo and dryness), each on a scale ranging from 0 (none) to 3 (severe). Investigator or designee also asked the subjects to evaluate their symptoms of pruritus and sleep loss (average for the last 3 days/nights) on a VAS from 0 to 10. The SCORAD score was calculated in the CRF.

Pruritus numeric rating scale (NRS). Pruritus NRS is a scale used by the subjects to report the intensity of their pruritus (itch) during the last 24 hours. Pruritus NRS has been validated in other AD clinical studies in adults and the minimum clinically important difference was shown to be 3 to 4.

The screening PP NRS score, measuring maximal pruritus intensity, was determined by a single PP NRS assessment (score ranging from 0 to 10) for the 24-hour period immediately preceding the screening visit. The baseline PP NRS score was determined based on the average of daily PP NRS scores (score ranging from 0 to 10) during the 7 days immediately preceding baseline (rounding was not permitted). A minimum of 4 daily scores out of the 7 days immediately preceding baseline was required for this calculation. Subjects received instructions on how to use and record their pruritus NRS scores on an electronic device and completed the assessment once daily in the evening throughout the clinical study (including the run-in and the follow-up period). Subjects were asked the following questions in their local language:
For average itch intensity: “On a scale of 0 to 10, with 0 being ‘no itch’ and 10 being ‘worst itch imaginable’, how would you rate your itch overall during the previous 24 hours?”
For maximum itch intensity: “On a scale of 0 to 10, with 0 being ‘no itch’ and 10 being ‘worst itch imaginable’, how would you rate your itch at the worst moment during the previous 24 hours?”

Application of topical medication for Atopic Dermatitis: Diary. Subjects were instructed to use the authorized background TCS as prescribed by the investigator. Each evening, subjects were asked to record the response (yes/no) to the following question, “Did you apply the eczema medication your doctor gave you, to your skin today?”, on an electronic device (i.e., diary), throughout the clinical study (including the run-in and the follow-up period).

Sleep disturbance NRS. Sleep disturbance NRS is a scale used by the subjects to report the degree of their sleep loss related to AD. Subjects received instructions on how to use and record their sleep disturbance NRS scores on an electronic device, and completed the assessment once daily in the morning throughout the clinical study (including the run-in and the follow-up period). Subjects were asked the following questions in their local language: on a scale of 0 to 10, with 0 being ‘no sleep loss related to signs/symptoms of AD’ and 10 being ‘I cannot sleep at all due to the signs/symptoms of AD’, how would you rate your sleep last night?”

Dermatology life quality index/Children's Dermatology life quality index. Dermatology Life Quality Index (DLQI) is a validated 10-item questionnaire for subjects aged >16 years, covering domains including symptoms/feelings, daily activities, leisure, work/school, personal relationships and treatment. Children's DLQI (cDLQI) is a comparable validated 10-item questionnaire designed for pediatric subjects aged 16 years or less. Subject rated each question ranging from 0 (not at all) to 3 (very much). A higher total score indicates a poorer QoL.

Safety assessments was conducted for all subjects at the screening visit (upon signing of the ICF) and at every subsequent visit. An adverse event (AE) is defined as any untoward medical occurrence in a clinical study subject administered a medicinal product which does not necessarily have a causal relationship with this treatment. An AE can therefore be any unfavorable and unintended sign (including an abnormal laboratory finding), symptom, or disease temporally associated with the use of a medicinal (investigational) product, whether or not it is related to the medicinal (investigational) product. This includes an exacerbation of pre-existing conditions or events, intercurrent illnesses, drug interaction or the significant worsening of the indication under investigation that is not recorded elsewhere in the CRF under specific efficacy assessments. Each AE was assigned a category by the investigator as follows:
Mild: An AE that is easily tolerated by the subject, causes minimal discomfort and does not interfere with everyday activities.
Moderate: An AE that is sufficiently discomforting to interfere with normal everyday activities; intervention may be needed.
Severe: An AE that prevents normal everyday activities; treatment or other intervention usually needed.

If there is a change in severity of an AE, it must be recorded as a separate event.

An adverse event of special interest (AESI) was a noteworthy event for the study drug that should be monitored closely and reported immediately. An AESI was either serious or non-serious. Based on the potential risks of nemolizumab and the risks associated with biologics (and their biosimilar equivalents) in general (i.e., class effects), the following AEs will be considered AESIs:
Injection-related reactions including anaphylactic reactions, acute allergic reactions requiring treatment, and severe injection site reaction (ISR) (i.e., lasting >24 hours)
Newly-diagnosed asthma or worsening of asthma
   More specifically, subjects with a medical history of asthma was referred to the physician who managed their asthma when:
      ACT score≤19; an ACT score≤19 conveyed asthma that was not adequately controlled. An AESI was reported based on the investigator's clinical judgment of the managing physician's report.
      PEF<80% of the predicted value; an AESI was reported.
      Unexpected worsening of asthma was observed or reported. An AESI was reported based on the investigator's clinical judgment.
   Subjects without a medical history of asthma were referred to an appropriate respiratory physician/specialist when:
      Signs and/or symptoms suggestive of asthma have been observed or reported. An AESI was reported based on the investigator's clinical judgment of the specialist's report.
      Respiratory assessment suggests a decline in the subject's respiratory health. An AESI was reported based on the investigator's clinical judgment of the specialist's report.
Infection. Any severe infection, or any infection requiring treatment with parenteral antibiotics, or oral antibiotics/antivirals/antifungals for >2 weeks
Peripheral edema: limbs, bilateral
Facial edema
Elevated ALT or AST (>3×ULN) in combination with elevated bilirubin (>2×ULN)

Serious adverse event (SAE). An SAE is any untoward medical occurrence or effect that, at any dose, (1) results in death; (2) is life-threatening; (3) requires or prolongs inpatient hospitalization; (4) results in persistent or significant disability/incapacity; (5) results in a congenital anomaly/birth defect; or (6) an important medical event that may not result in death, be life-threatening, or require hospitalization, may be considered an SAE when, based upon appropriate medical judgment, the event may jeopardize the safety of the subject, and may require medical or surgical intervention to prevent one of the outcomes listed above in this definition (e.g., intensive treatment in an emergency room or at home for allergic bronchospasm, blood dyscrasia, or convulsions that do not result in hospitalization).

Unexpected adverse reactions. An unexpected adverse reaction is any untoward and unintended response that is related to the administration of the study drug at any dose, the nature or severity of which is not consistent with the applicable product information (e.g., reference safety information in the investigators brochure for nemolizumab, study protocol, etc.).

Clinical laboratory evaluations. The hematology, chemistry laboratory analyses, and urinalyses will be performed at a central laboratory as specified in Table 7. Pregnancy testing, Tuberculosis screening, complete physical examination (PE), respiratory assessments, asthma control test (ACT), respiratory examination, spirometry/peak expiratory flow, and a 12-lead electrocardiogram (ECG) were performed. Vital signs, height and weight were monitored.

Pharmacokinetics:

Blood sampling. Blood samples were collected according to Table 7 to determine the PK profile of nemolizumab. The serum concentration of nemolizumab was assessed at baseline, Weeks 1 to 2, 4, 8, 12, 16, 24, and at any unscheduled visit for safety reasons. At each sampling time point for PK assessment, the collected blood was placed to clot at room temperature (no more than 60 minutes after collection) and then centrifuged at room temperature. The serum was collected into storage tubes. At the study drug injection visits (baseline, Weeks 4, 8, and 12), PK samples were collected within 30 minutes before study drug injection (predose samples) at Weeks 1 to 2, 16, and 24, attempts were made to collect PK samples at approximately the same time of the day, to the extent possible. The date and the time of each sample collection was recorded in the CRF, together with the time of study drug injection at the same visit (or missed injection if applicable). Processing of blood samples for PK assessments were described in the laboratory manual.

Nemolizumab (CD14152) quantification in serum. Concentration of nemolizumab (CD14152) in the serum were determined by the designated CRO (Shin Nippon Biomedical Laboratories, Ltd, Japan) using a validated enzyme-linked immunosorbent assay (ELISA) method with a limit of quantification of 100 ng/mL.

Pharmacokinetic analysis. Pharmacokinetic parameters of nemolizumab in the serum were calculated by the designated CRO (Certara) using the following 2 analyses: non-linear mixed effect modeling and noncompartmental analysis (NCA) analysis.

Analysis 1:

A population PK (popPK) model was used to describe the time course of nemolizumab exposure and to investigate sources of variability in subject exposure. A first-order absorption and a 1-compartment distribution popPK model was developed using nemolizumab serum concentrations from 3 clinical studies conducted in adults (CIM001JP, CIM003JG, and SPR.114322) and used to derive empirical Bayes estimates in the adolescent population based on their baseline characteristics, their dosing history and their measured concentrations.

The adequacy of the model to properly describe the adolescent data were based on the model diagnostic tools described in a dedicated PK modeling plan. Estimates of main popPK parameters (CL/F, V_{d}/F, T_{lag}, kₐ), including inter-individual variability, covariate effects, residual error and their relative standard error [RSE]), were obtained using the popPK model.

Other individual nemolizumab PK parameters (Cₘₐₓ, Tₘₐₓ, C_{trough}, AUC_{0-4 w}, AUC_{0-8 w}, AUC_{0-12 w}, AUC_{0-16 w}, AUC_{inf}, and t_{1/2}) were derived using the same popPK model.

Analysis 2:

Some individual nemolizumab PK parameters (AUC_{0-4 w}, AUC_{0-8 w}, AUC_{0-12 w}, and AUC_{0-16 w}) also were calculated using NCA using only the serum concentrations actually observed in the study.

Pharmacokinetic/Pharmacodynamic analysis (PK/PD). Pharmacokinetic/PD models were developed to correlate the concentration of nemolizumab to clinical efficacy. The popPK model combined the following pharmacodynamic (PD) endpoints: eczema area and severity index (EASI), investigator's global assessment of dermatitis severity (IGA), and weekly average Peak Pruritus Numeric Rating Scale (PP-NRS). For each subject, the concentration time-course was predicted up to the last PD assessment time based on individual Bayes posthoc estimates of the PK parameters and was used as a driver in the PD models.

Other Variables:

Immunogenicity. Blood samples were collected according to Table 7 to assess ADA. Serum samples were assessed for ADA at Baseline, Weeks 4, 8, 16, 24, and at any unscheduled visit for safety reasons. ADA were determined at these time points by the designated CRO (LSI Medience Corporation. Japan) using a validated ELISA assay. The serum concentration were assessed using a multitiered approach.

If serum circulating ADA were detected in the screening assay, presence was confirmed and characterized (e.g., titer and neutralizing potential) using a validated assay. Incidence of positive ADA results were summarized (absolute occurrence and percent of subjects).

For ADA assessment, blood was collected at the same time of PK sampling according to Table 7. Additional visits (considered as unscheduled visits) after the planned last study visit (12 weeks after the last study drug injection) may have been conducted to collect additional samples for further analysis of ADA. Processing of blood samples for PK and ADA assessments were well known in the art.

Biomarkers.

Blood and stratum corneum samples were collected to investigate the effect of nemolizumab on selected biomarkers. Samples were shipped to the designated CRO for biomarker assessment, unless otherwise specified. The biomarkers samples (plasma and skin) were destroyed after all tests were completed and when the pharmacodynamics report was approved.

Blood samples were collected for assessment of TARC and immunoglobulin E (IgE) by the central laboratory. Blood samples were collected for plasma biomarker assessment according to Table 7. Aliquots of plasma sample were shipped to the designated CRO for assessment using a set of biomarkers relevant for AD, including but not limited to IL-6, IL-8 and IL-18. Depending on results with protein biomarkers, the quantity of samples left over, and the compatibility of the initially collected samples with further investigations, the expression of micro ribonucleic acid (miRNA) and messenger ribonucleic acid (mRNA) markers were analyzed subsequently.

Stratum corneum (SC) samples were collected using tape strips (D-squames) according to Table 7. At baseline before administration of study drug, 2 lesional and 2 non-lesional areas were identified and 4 rectangular D-squames (2.3 cm×3.5 cm in dimension) were collected from each area, making 16 D-squames in total (8 from lesional skin and 8 from non-lesional skin). At Week 16, 4 rectangular D-squames were collected from the 2 lesional areas identified at baseline (8 D-squames in total). Aliquots of samples were shipped to the designated CRO for analysis of selected protein biomarkers relevant for AD (including TARC, IL-6, IL-8, IL-18, etc.). In addition, the levels of end-of-study (EOS) ceramides were quantified. Depending on results with protein and ceramide biomarkers, the quantity of samples left over, and the compatibility of the initially collected samples with further investigations, the expression of lipids and of miRNA and mRNA markers could be subsequently analyzed.

Data Analysis and Presentation.

Pharmacokinetic concentration presentation. Nemolizumab serum concentration data (unit: ng/mL) were summarized by visit from baseline to Week 24 using the following statistics: n, arithmetic mean, standard deviation, % CV, geometric mean, % CV geometric mean, median, minimum, maximum, 95% CI of mean and number of BLQs (Below Limit of Quantification). BLQ was considered as missing and excluded from the descriptive summary.

Individual serum concentration and mean concentration were plotted by visit on both a linear and semi-logarithmic scale on the PK population (unit for x axis: week). For individual concentration plot, BLQ was considered as missing. For mean concentration plot, mean concentration not calculated were considered as missing. In addition, individual serum concentration were plotted over time with different color for subjects with treatment-related ADA+ (ADA− at baseline and ADA+after treatment), ADA+at baseline and ADA− from baseline during the study). Individual serum concentrations were listed and non-quantifiable concentration were listed as BLQ (<100 ng/mL).

Pharmacokinetic parameters presentation. Estimates of main population PK parameters (Cl/F, V_{d}/F, T_{lag}, kₐ), including inter-individual variability, covariate effects, residual error and their RSE, are presented in a dedicated PK report. Individual derived PK parameters at steady state estimated with the popPK model and individual observed PK parameters at steady state calculated with the NCA were summarized using the following statistics: n, arithmetic mean, standard deviation, geometric mean, % CV, median, minimum, maximum and 95% CI. In addition, the comparison of the average observed trough (C_{trough}) nemolizumab concentration between ADA+ samples and ADA− samples were performed using (analysis of variance) ANOVA and the comparison results were presented.

Efficacy Variables:

All the efficacy analyses were to be based on the intent-to-treat (ITT) population. All efficacy variables were to be summarized by analysis visit from baseline unless specified otherwise. For efficacy analysis, unscheduled visit or ET visit data were windowed based on analysis visit window to be considered for by-visit summary. All efficacy analysis were to be performed using last observation carried forward (LOCF) or Nonresponder imputation (unless stated otherwise) and also performed with OCs as sensitivity analysis

Eczema Area and Severity Index (EASI) scores were derived and recorded in the CRF as described in Table 8. The EASI was summarized descriptively by visit using LOCF and OC. Absolute change and percent changes from Baseline were included in the analysis. For the LOCF analysis, the imputation was performed for the EASI overall imputed score. Percent change in EASI using LOCF was displayed graphically.

TABLE 8
Eczema area and severity index score calculation

| Body region | EASI score |
|---|---|
| Head/Neck | (Erythema + Induration/Papulation + |
| | Excoriation + Lichenification) × Area × 0.1 |
| Upper limbs | (Erythema + Induration/Papulation + |
| | Excoriation + Lichenification) × Area × 0.2 |
| Trunk | (Erythema + Induration/Papulation + |
| | Excoriation + Lichenification) × Area × 0.3 |
| Lower limbs | (Erythema + Induration/Papulation + |
| | Excoriation + Lichenification) × Area × 0.4 |
| EASI Overall | Sum of the above 4 body region scores |

The investigator's global assessment (IGA) is a 5-point scale used by the Investigator to evaluate the global severity of AD and the clinical response, as described in Table 9. IGA score and change from Baseline were summarized by visit using LOCF and OC. IGA success was defined as 0 (clear) or 1 (almost clear) and at least a 2-grade improvement change from Baseline. The IGA treatment success rate was obtained as the ratio between the number of subjects achieving IGA treatment success and the number of subjects with available IGA results at the visit, and were also summarized by visit from Baseline using Non-responder, LOCF, and OC. The proportion of subjects who achieved IGA success using Non-responder were displayed graphically.

TABLE 9
Investigator's global assessment 5-point scale

| Status | Score | Definition |
|---|---|---|
| Clear | 0 | Minor, residual hypopigmentation/ |
| | | hyperpigmentation, no erythema or induration/ |
| | | papulation, no oozing/crusting. |
| Almost | 1 | Trace faint pink erythema, with barely perceptible |
| clear | | induration/papulation and no oozing/crusting. |
| Mild | 2 | Faint pink erythema with mild induration/ |
| | | papulation and no oozing/crusting. |
| Moderate | 3 | Pink-red erythema with moderate induration/ |
| | | papulation with or without oozing/crusting. |
| Severe | 4 | Deep or bright red erythema with severe |
| | | induration/papulation with oozing/crusting. |

The body surface area (BSA) of AD involvement was assessed as a percentage for 8 different parts of the body and were reported individually and as an overall BSA (calculated with the sum of all parts combined), as derived in the CRF. The body parts assessed, and their highest possible scores were as follows: head and neck [9%], anterior trunk [18%], back [18%], upper limbs [18%], lower limbs [36%], and genitals [1%]. Overall BSA of AD involvement was summarized descriptively by visit as continuous variables using LOCF and OC. Absolute change and percent from baseline were also included in the descriptive analysis. For the LOCF analysis, the imputation was performed separately for each body part, and the imputed overall BSA was obtained with the sum of the imputed body region scores.

Scoring Atopic Dermatitis (SCORAD) is a validated measure commonly used in clinical studies and clinical practice to assess the severity and the extent of AD signs and symptoms. SCORAD takes into account the extent and intensity of 6 types of basic lesions (erythema/darkening, edema/papule, oozing/crusting, excoriation, lichenification/prurigo and dryness) and symptoms (itching and loss of sleep). SCORAD ranges from 0 to 103 and has 3 components: extent, intensity, and subjective symptoms. Calculation of SCORAD were performed as follows: the extent of BSA affected by AD were the “extent of area involved component” (denoted with A). Next, the intensity of all basic types of lesions were assessed. Depending on the intensity, each of these is assigned a score of 0 (absence), 1 (mild), 2 (moderate) or 3 (severe). The sum of these scores were used to estimate the “intensity component” (denoted with B). Finally, based on a VAS (recorded in centimeters), the presence of irritability and loss of sleep for the past 3 nights were evaluated as a value between 0 (no symptoms) and 10 (worst symptoms ever had). The sum of the 2 values were used to estimate the “subjective symptoms component” (denoted with C). The SCORAD total score was then calculated as=A/5+(7*B)/2+C. SCORAD was summarized descriptively by visit using LOCF and OC. Absolute change and percent changes from baseline were also included in the summary. For the LOCF analysis, the imputation was performed separately for each SCORAD component, and the imputated total score were obtained with the imputed components.

The Pruritus NRS was used by the subjects to report the intensity of their pruritus (itch) during the last 24 hours (subject-reported assessments of pruritus were collected daily on a diary. The scale for average itch intensity and maximum itch intensity has values in a range between 0 to 10, with 0 being ‘no itch’ and 10 being ‘worst itch imaginable’. Average and PP-NRS scores were obtained based on the average and maximum daily scores during the 7 days immediately preceding each scheduled visit (rounding was not permitted), and were denoted as “weekly” scores (average weekly and peak weekly, respectively). A minimum of 4 daily scores were required for these calculations, and if less than 4 daily scores were available the respective weekly score was a missing value. Weekly average of peak pruritus (PP) NRS and weekly average of average pruritus (AP) NRS scores were summarized descriptively by visit as continuous variables using LOCF and OC. Absolute and percent changes from baseline were also included in the analysis. Absolute and percent change from baseline in weekly average of peak pruritus NRS and weekly average of average pruritus NRS using LOCF were displayed graphically. For the LOCF analysis, the imputation was performed separately for each weekly average or peak pruritus NRS score.

Sleep disturbance NRS is a scale to be used by the subjects to report the degree of their sleep loss related to AD. The scale has values in a range between 0 to 10, with 0 being ‘no sleep loss related to signs/symptoms of AD’ and 10 being ‘I cannot sleep at all due to the signs/symptoms of AD. Subject-reported assessments of sleep disturbance NRS were collected daily in a diary. Weekly sleep disturbance NRS scores were obtained based on the average daily values during the 7 days immediately preceding each scheduled visit. A minimum of 4 daily scores were required for this calculation, and if less than 4 daily scores were available the respective weekly score were a missing value. Weekly average sleep disturbance NRS were summarized descriptively by visit as continuous variables using LOCF and OC. Absolute and percent changes from baseline were also included in the analysis. Absolute and percent change from baseline in weekly average sleep disturbance NRS using LOCF were displayed graphically. For the LOCF analysis, the imputations were performed for each weekly average sleep disturbance NRS score.

Application of topical medication for atopic dermatitis. Subject-reported assessments of the use of topical AD medication for their eczema were collected daily on a diary. Subjects were instructed to use the authorized background TCS, and were asked to record the response (yes/no) to the following question, “Did you apply the eczema medication your doctor gave you, to your skin today?” on an electronic device (i.e., diary), throughout the clinical study (including the run-in and the follow-up period). A topical AD medication-free day was a day without use of topical AD medication. For each subject, each day was classified as free or not. Days of topical AD medication use were calculated by summing up the days recorded as yes on a diary. The daily values in visit interval were used for calculation of visit information. Topical AD medication-free days in each visit interval were calculated as follows: topical AD medication-free days in visit interval=Days in the interval-Days of topical AD medication use in the interval. The number of topical AD free days were summarized descriptively by visit interval as continuous variables. No imputation was used.

The dermatology life quality index (DLQI) is a validated 10-item questionnaire for subjects aged >16 years, covering domains including symptoms/feelings, daily activities, leisure, work/school, personal relationships and treatment. DLQI data were recorded in the CRF. Children's DLQI (cDLQI) is a comparable validated 10-item questionnaire designed for pediatric subjects and cDLQI was used if the subjects were aged 16 years or less. Each question in DLQI/cDLQI has values in a range between 0 (not at all) and 3 (very much) as defined in Table 10. The DLQI/cDLQI total score was calculated by summing the score of each question resulting in a maximum score of 30 and a minimum of 0. The higher the score, the more QoL was impaired. DLQI and cDLQI scores and their change from baseline were summarized descriptively by visit as continuous variables.

TABLE 1
Dermatology life quality index and children's
dermatology life quality index question point score

| Score | DLQI | cDLQI |
|---|---|---|
| 0 | Not at all | Not at all |
| 1 | A little | Only a little |
| 2 | A lot | Quite a lot |
| 3 | Very much | Very much |

Safety Variables:

Extent of Exposure. The extent of exposure (treatment duration, total dose administered, planned dose and treatment compliance percentage) were summarized. Treatment duration (in days) was calculated as follows: [(date of last treatment—date of first treatment)+1]. Treatment compliance were summarized based on the treatment records and drug dispensation logs recorded in the CRF. The compliance percentage were calculated as: (Dose volume administered)/(Dose volume planned)×100, with, the planned dose of 60 mg on Day 1, followed by injections of 30 mg every 4 weeks for 12 weeks. Once the dosing solution is prepared, 1.0 mL of it were drawn into a syringe, which were administered subcutaneously in the abdomen of the subjects as planned dose. Adverse events. Adverse events were coded using the Medical Dictionary for Regulatory Activities (MedDRA) Version 21.1. Summaries of AEs included only treatment emergent AEs (TEAEs) defined as AEs with an onset date/time on or after the date/time of first treatment. AEs with completely missing onset dates/time were considered as TEAEs. If AE onset date/time was partially missing, the AE was considered as TEAE unless the non-missing part of date/time proved it started before the first treatment date/time.

By-visit summaries will use the analysis visit. Unscheduled and early termination visits will be windowed based on the analysis visit window defined in the SAP. Daily diary efficacy data (pruritus and sleep disturbance NRS) were classified into analysis visits as follows considering the data during the 7 days immediately preceding each scheduled visit. Table 11 shows the diary data windows by week:

TABLE 11
Diary data windows by week

| | Target | Diary |
|---|---|---|
| Analysis Visit | Study Day | Data Window |
| Baseline | 1 | −7 to −1 |
| Week 1-2 | 10 | 3 to 9 |
| Week 4 | 29 | 22 to 28 |
| Week 8 | 57 | 50 to 56 |
| Week 12 | 85 | 78 to 84 |
| Week 16 | 113 | 106 to 112 |
| Week 24 | 169 | 162 to 168 |
| Follow-up | | |

Daily topical AD medication used data were classified into visit intervals as follows (Table 12):

TABLE 12
Visit intervals for daily topical atopic dermatitis medication

| | Target | Diary |
|---|---|---|
| Analysis Visit Interval | Study Day | Data Window |
| Baseline to Week 1-2 | 10 | 1 to 9 |
| Week 1-2 to Week 4 | 29 | 10 to 28 |
| Week 4 to Week 8 | 57 | 29 to 56 |
| Week 8 to Week 12 | 85 | 57 to 84 |
| Week 12 to Week 16 | 113 | 85 to 112 |
| Week 16 to Week 24 follow-up | 169 | 113 to 168 |

Results

Demographic and Other Baseline Characteristics.

As described in Table 13, a total of 31 subjects were screened with 11 screen failures due to either inclusion/exclusion criteria not met or subject request. Consequently, 20 subjects were enrolled. Eighteen subjects (90.0% of those enrolled) were treated at least once. Two subjects (8238-003 and 8664-001) who did not meet the inclusion criteria and were screen failures, were enrolled in error. No treatment was administered for either of these subjects. Of those 18 subjects who were treated at least once, 13 subjects (65.0% of those enrolled) received all treatment doses and were classified as completers, while 5 subjects (25.0% of those enrolled) discontinued treatment early. Early discontinuation of treatment was due to AEs in 3 subjects (15.0% of those enrolled) and protocol deviations in 2 subjects (10.0% of those enrolled). Among the subjects who discontinued treatment early, 2 subjects (8203-005 and 8668-001) completed all study visits up to Week 24, while 3 subjects (8614-001, 8614-002, and 8667-001) exited the study before the final visit. Overall. 15 subjects (75% of those enrolled) completed the study (Week 24 visit), while 5 subjects (25.0% of those enrolled), including those subjects enrolled in error, discontinued the study early.

TABLE 13
Disposition of subjects

| | Nemolizumab | |
|---|---|---|
| | 30 mg n (%) | |
| Summary of screen failures | | |
| Subjects screened | 31 | |
| Screen failure | 11 | |
| Reason for screen failure | | |
| Inclusion/Exclusion criteria not met | 10 | (90.9) |
| Subject's request | 1 | (9.1) |
| Number of subjects enrolled | 20 ^{a} | |
| Enrolled and treated at least once | 18 | (90.0) |
| Treated at least once and reached | 15 | (75.0) |
| Week 24 visit | | |
| Did not receive all doses | 2 | (10.0) |
| Received all doses (i.e., completers): | 13 | (65) |
| Number of subjects who completed treatments | 13 | (65.0) |
| Number of subjects who discontinued treatment | 5 | (25.0) |
| Primary reason for discontinuation of treatment | | |
| Adverse event | 3 | (15.0) ^{b} |
| Protocol deviation | 2 | (10.0) |
| Number of subjects who completed the study | 15 | (75.0) |
| Number of subjects who discontinued from the | 5 | (25.0) |
| study | | |
| Primary reason for discontinuation | | |
| from the study | | |
| Subject's request | 1 | (5.0) |
| Protocol Deviation | 2 | (10.0) |
| Other: Enrolled in error | 2 | (10.0) |

There were 12 (60.0%) subjects with at least 1 major protocol deviation. Most major protocol deviations were reported for study procedure/other (5 [25.00%]) and inclusion or exclusion criteria (4 [20.00%]). Major protocol deviations are summarized Table 14.

TABLE 14
Major protocol deviations (Intent-to-treat population)

| | Nemolizumab | |
|---|---|---|
| | 30 mg | |
| | N = 20 | |
| Major Protocol Deviations | n (%) | |
| Subjects with at least one major | 12 | (60.0) |
| protocol deviation | | |
| Study Procedure/Other | 5 | (25.0) |
| Inclusion or Exclusion Criteria | 4 | (20.0) |
| Informed Consent/Other | 2 | (10.0) |
| Study Procedure/Missed procedure | 2 | (10.0) |
| Informed Consent/ICF not signed or | 1 | (5.0) |
| signed late | | |
| Study Procedure (non-subject) | 1 | (5.0) |
| Study Procedure/Visit Missing | 1 | (5.0) |

Among the ITT population, the mean age of all 20 subjects was 14.8±1.59 years (Min-Max: 13-17 years), with body weight ranging from 49 to 112 kg. Overall, 5000 (N=10) were in the 12-14 years of age group and 50% were in the 15-17 years of age group (N=10). Among the ITT population, 60% of the subjects were female. Almost all subjects were Black or African American (50.0%), or White (35.0); 80% of subjects were not Hispanic or Latino. The demographic and baseline characteristics of all the 20 subjects are presented in Table 15.

TABLE 15
Demographic and baseline characteristics
(Intent-to-treat population)

| | Nemolizumab | |
|---|---|---|
| | 30 mg (N = 20) | |
| Gender [n (%)] | | |
| Male | 8 | (40.0) |
| Female | 12 | (60.0) |
| Ethnicity [n (%)] | | |
| Hispanic or Latino | 4 | (20.0) |
| Not Hispanic or Latino | 16 | (80.0) |
| Race [n (%)] | | |
| Asian | 2 | (10.0) |
| Black or African | 10 | (50.0) |
| American | | |
| White | 7 | (35.0) |
| Other | 1 | (5.0) |
| Age (years) | | |
| Mean (SD) | 14.8 | (1.59) |
| Median | 14.5 | |
| Min-Max | 13-17 | |
| Age group n (%) | | |
| 12-14 years | 10 | (50.0) |
| 15-17 years | 10 | (50.0) |
| Weight (kg) | | |
| Mean (SD) | 69.07 | (17.47) |
| Median | 64.1 | |
| Min-Max | 49.0-112.0 | |
| BMI (kg/m²) | | |
| Mean (SD) | 25.4 | (5.66) |

| | |
|---|---|
| Median | 24.6 |
| Min-Max | 17.24-35.95 |

As presented in Table 16, at Baseline, the mean (SD) baseline EASI score was 25.2 (7.22) for the ITT population. All subjects had moderate or severe IGA scores at Baseline; moderate scores were reported for 15 (751) subjects and severe for 5 (250) subjects. The mean percent BSA involvement (SD) at Baseline was 41.3 (12.6). The mean (SD) for weekly average of PP NRS and weekly average of AP NRS at Baseline was 6.9 (1.66) and 6.2 (1.7), respectively. The mean (SD) weekly sleep disturbance NRS at Baseline was 5.6 (2.27). The mean (SD) SCORAD scores was 63.0 (11.34). Mean (SD) scores for DLQI and cDLQI at Baseline were 11.8 (3.1) and 10.6 (5.42), respectively.

TABLE 16
Summary of baseline disease characteristics
(Intent-to-treat population)

| | Nemolizumab | |
|---|---|---|
| | 30 mg (N = 20) | |
| EASI Score | | |
| n | 20 | |
| Mean (SD) | 25.2 | (7.22) |
| Median | 23.6 | |
| Min-Max | 16.30-37.46 | |
| IGA Score, n (%) | | |
| Clear | 0 | |
| Almost Clear | 0 | |
| Mild | 0 | |
| Moderate | 15 | (75.0) |
| Severe | 5 | (25.0) |
| BSA % involvement of AD | | |
| n | 20 | |
| Mean (SD) | 41.3 | (12.6) |
| Median | 39.5 | |
| Min-Max | 23-60- | |
| Weekly average of peak pruritus NRS | | |
| n | 18 | |
| Mean SD) | 6.9 | (1.66) |
| Median | 7.5 | |
| Min-Max | 4.00-9.43 | |
| Weekly average of average pruritus NRS | | |
| n | 18 | |
| Mean (SD) | 6.2 | (1.7) |
| Median | 6.155 | |
| Min-Max | 3.33-8.43 | |
| Weekly sleep disturbance NRS | | |
| n | 18 | |
| Mean (SD) | 5.6 | (2.27) |
| Median | 5.9 | |
| Min-max | 0.63-8.88 | |
| SCORAD | | |
| n | 20 | |
| Mean (SD) | 63.0 | (11.34) |
| Median | 60.8 | |
| Min-Max | 45.2-82.3 | |
| DLQI | | |
| n | 5 | |
| Mean (SD) | 11.8 | (3.1) |
| Median | 11.0 | |
| Min-max | 8-15 | |
| cDLQI | | |
| n | 13 | |
| Mean (SD) | 10.6 | (5.42) |

| | |
|---|---|
| Median | 9.0 |
| Min-max | 3-21 |

BSA = Body Surface Area;
cDLQI = Children's Dermatology Life Quality Index;
DLQI = Dermatology Life Quality Index;
EASI = Eczema Area and Severity Index;
IGA = Investigator's Global Assessment;
Max = maximum;
Min = minimum;
NRS = numerical rating scale;
SCORAD = Summary of Scoring Atopic Dermatitis;
SD = standard deviation
Note:
N = number of subjects in the population;
n = number of subjects with available data
Baseline was the last available measurement prior to or on the first treatment. If date of treatment was missing, the date of enrollment was used instead.
Percentage (%) was calculated using the number of subjects in population as the denominator.

Medical history. All 20 subjects in the ITT population had at least 1 medical condition recorded in their medical history (Table 14.1.3.2.1). Of these, 10 subjects (50.0%) had asthma. A history of cellulitis and anaemia was reported by 2 subjects and pneumonia and staphylococcal infection was reported by 1 subject each. One subject had a history of methicillin sensitive Staphylococcus aureus (MSSA+) but it had resolved before the subject was enrolled in the study. No prior or concomitant medical and surgical procedures were recorded. Nineteen (95%) subjects used at least 1 prior medication, all of whom used dermatological corticosteroids. The most commonly used prior dermatological corticosteroids were triamcinolone (11 [55.0%]) and hydrocortisone (7 [35.0%]). Of the 19 (95%) subjects who used at least 1 background TCS, all used mometasone furoate 0.1% cream, 18 (90.0%) subjects used %]), hydrocortisone 1% cream, and 6 (30.0%) subjects used hydrocortisone butyrate 0.1% cream. One subject used at least 1 rescue medication, which were. Systemic corticosteroids betamethasone and triamcinolone) and a topical corticosteroid preparation (betamethasone valerate 0.1% lotion).

Pharmacokinetic Results

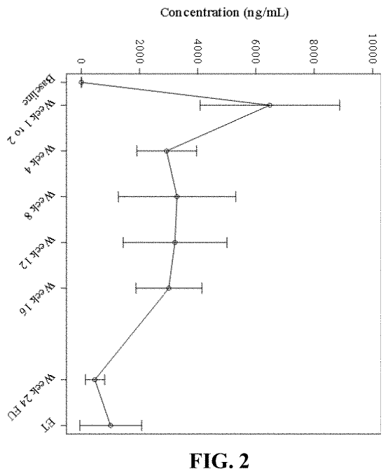

Observed concentrations and Non-compartmental analysis. Nemolizumab maximum serum concentration (Cₘₐₓ) were observed 1 or 2 weeks after the first subcutaneous injection of the 60-mg LD (FIG. 2Error! Reference source not found.). The maximal concentrations ranged from 2550 ng/mL to 11100 ng/mL, with a mean value of 6532.9±2329.9 ng/mL. Steady-state condition was achieved by Week 4, with mean observed trough concentration ranging from 2935.3±1029.4 ng/mL to 3292.3±2017.8 ng/mL at steady state. The mean observed trough concentrations from Week 4 to 16 are reported in Table 17 together with the correspondent model-predicted values. At the end of the treatment period (week 16), the drug was eliminated from the serum. An NCA was performed on the PK population. Mean (±SD) AUC values calculated from the observed pre-dose samples at 0 to 4, 8, 12, and 16 weeks were, respectively, 122.3±44.1 μg*day/mL, 233.3±80.1 μg*day/mL, 332.9±121.7 μg*day/mL and 372.9±149.9 μg*day/mL. Due to the limited number of PK samples collected at the end of the treatment period, the terminal half-life cannot be accurately calculated from NCA analysis

TABLE 17
Mean (±SD) nemolizumab serum trough concentrations (ng/mL)
observed and predicted in adolescents (PK population, N = 18)

| | Concentration (C_{trough}) | Concentration (C_{trough}) |
|---|---|---|
| | observed in | predicted in |
| Week | adolescents ^{a} (ng/mL) | adolescents ^{b} (ng/mL) |
| 4 | 2935.3 (1029.4) | 3271.9 (1268.6) |
| 8 | 3292.3 (2017.8) | 3056.5 (1367.9) |
| 12 | 3221.6 (1781.2) | 2886.8 (1442.0) |
| 16 | 3010.0 (1131.8) | 2981.8 (1166.9) |

^{a.} Observed values from PK population
^{b.} Concentrations predicted with popPK model

Population pharmacokinetic analysis. An analysis was performed on the PK population, including all subjects in the safety population who provided at least 1 post-baseline drug concentration value above the LLOQ (≥100 ng/mL). Per the protocol, at least 6 subjects with a low body weight (i.e., ≤61 kg) were enrolled and included in the analysis.

The popPK model was built based on data from 407 adult subjects included in 3 clinical studies (CIM001JP, CIM003JG, and interim SPR.114322 data), which assessed the exposure to subcutaneous nemolizumab over the dose range of 0.1 to 3 mg/kg and 10 to 90 mg for weight-based and flat dosing, respectively. The effect of several covariates including age, body weight, serum creatinine, estimated glomerular filtration rate, bilirubin, serum albumin, total protein, IgE, sex, and clinical study was explored.

The PK of nemolizumab was described with a 1-compartment model with linear elimination and first-order absorption with a lag time. A moderate impact of the dose was identified on the bioavailability with an increase of bioavailability with decreasing dose. Body weight was identified as the main source of variability in the PK profile of nemolizumab and impacted both the CL/F and V/F. Indeed, an increase in body weight resulted in a decrease in nemolizumab systemic exposure. The albumin plasma concentrations also impacted CL/F. Estimates of the main population PK parameters in adults are reported in Table 18.

No impact of age was observed on estimates for systemic clearance, volume of distribution, constant of absorption or terminal half-life obtained with popPK modeling. All summary statistics in adolescents' Bayes estimates are in line with adult data, either model-derived (see Table 17 and Table 18) or observed in the Phase 2 study, SPR.114322.

Mean (±SD) model-predicted AUC values calculated from 0 to 4, 8, 12, and 16 weeks were, respectively, 144.1±50.8 μg*day/mL, 284.4±97.1 μg*day/mL, 411.7±142.9 μg*day/mL and 549.5±184.5 μg*day/mL.

TABLE 18
Summary of nemolizumab serum pharmacokinetics parameters
estimated with popPK analysis (PK population, N = 18)

| | | Mean (±SD) of | |
|---|---|---|---|
| | Population | individual | |
| | estimate | estimates in | |
| Parameter | in adults | adolescents | |
| CL/F (L/day) | 0.324 | 0.325 | (0.153) |
| Vz/F (L) | 7.46 | 7.15 | (2.24) |
| t_{1/2} (day) | 16.0 | 16.7 | (4.11) |
| kₐ (1/day) | 0.592 | 0.402 | (0.145) |
| Cₘₐₓ (ng/mL) | NC | 5975.5 | (2175.2) |
| AUC_{inf} (μg*day/mL) | NC | 111.4 | (48.0) |

NC: not calculated
CL/F: Apparent clearance,
V/F: Apparent volume of distribution;
Kₐ:Constant of absorption;
t_{1/2}: Terminal half-life;
Cₘₐₓ: peak concentration,
AUC_{inf}: Area under the Curve calculated to infinity

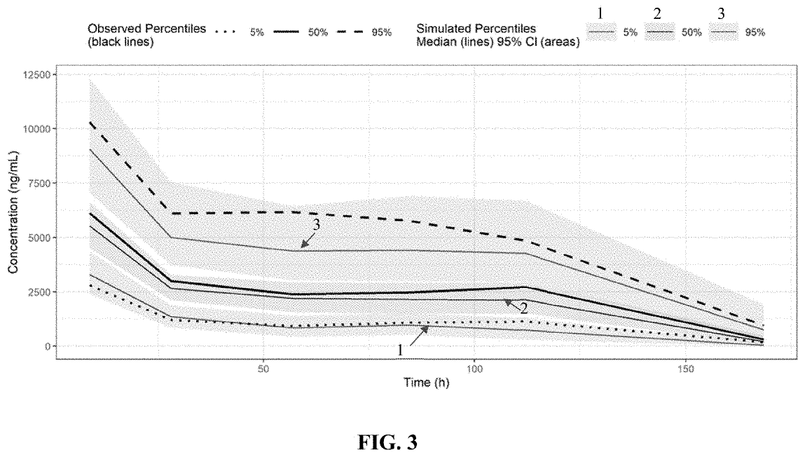

During the popPK analysis, the adult popPK model was applied to predict nemolizumab concentration data in adolescents, based on subjects' baseline characteristics and their dosing history. The predictions were then compared to the observed data (see Table 17, FIG. 2, and FIG. 3), showing that the adult model was able to accurately describe individual nemolizumab concentration-time profiles in adolescents too.

Pharmacokinetic/Pharmacodynamic Analysis

Individual predictions and empirical Bayes estimates generated from the popPK model were used to drive 3 PK/PD models, developed using adult data, for the following clinical endpoints: Eczema Area and Severity Index (EASI), Investigator's Global Assessment of dermatitis severity (IGA), and weekly average PP-NRS.

The EASI model was built based on data of 525 subjects with AD from 3 clinical studies (Studies CIM001JP, CIM003JG, and SPR.114322). This is a turnover model with inhibiting drug effect (sum of placebo effect and nemolizumab effect modeled as an Emax model) on the zero-order rate constant for production of response (Rin) and a combined proportional and additive residual error model. The parameter representing the drug concentration producing half of the maximal inhibiting effect (IC50) in the model was replaced by the parameter S0, computed as Imax/IC50, to stabilize the model. No significant covariate influenced model's estimates.

The PP-NRS model was built based on data of 225 subjects with AD from study SPR.114322. This is a turnover model with inhibiting drug effect (sum of placebo effect and nemolizumab effect modeled as an Emax model) on the zero-order rate constant for production of response (Rin) and a combined proportional and additive residual error model. No significant covariate influenced model's estimates.

The IGA model was built based on data of 486 subjects with AD from 2 clinical studies (Studies CIM003JG and SPR114322). This is a continuous-time Markov model that treated IGA scores as compartments and modelled the ascending and descending transitions between compartments. Baseline IGA score of 4 or 5 was found to be a significant covariate for all ascending parameters and the covariate effect was therefore implemented in the model.

The PK/PD models developed from adult data were applied to simulate the nemolizumab efficacy profile in adolescents, using subjects' characteristics (age, body weight, body mass index, etc.) and their dosing history. The predictions were then compared to the observed data.

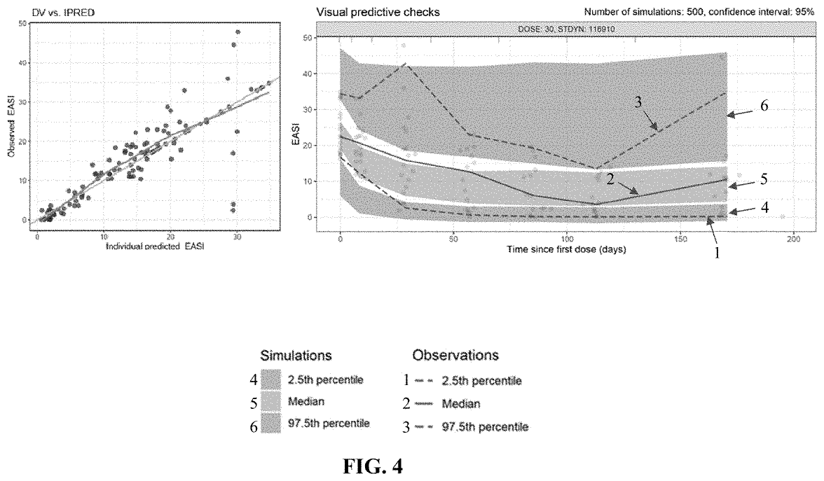

The EASI PK/PD model showed that the model structure is appropriate to describe adolescent data. The bias observed in the predictions of the observed data versus individual predicted scores for the higher ESAI scores are explained by the sparsity of the data (see FIG. 4).

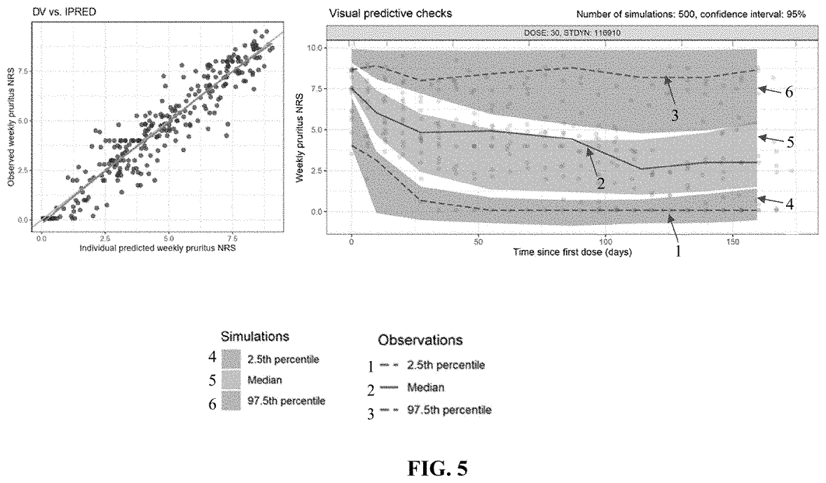

The PP-NRS PK/PD model showed that the model structure is appropriate to describe adolescent data (see FIG. 5).

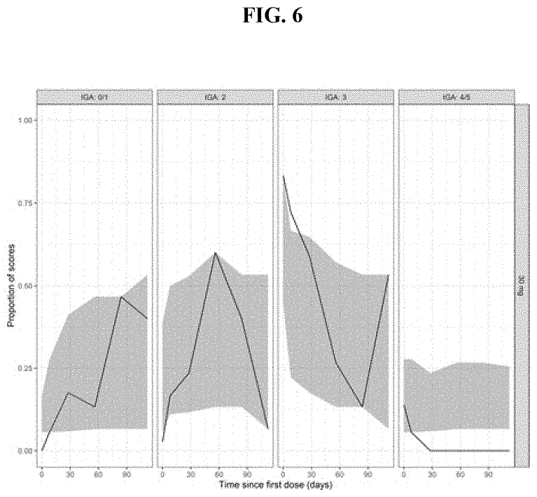

The IGA PK/PD model was adequate in its prediction, showing some discrepancy only for lumped score 4/5, possibly driven from lack of score 5 in adolescent (see FIG. 6).

Overall, the PK/PD models were able to accurately describe nemolizumab efficacy profiles in adolescents for the 3 clinical endpoints, thus confirming similarity of exposure-response in adults and adolescents.

Immunogenicity Results

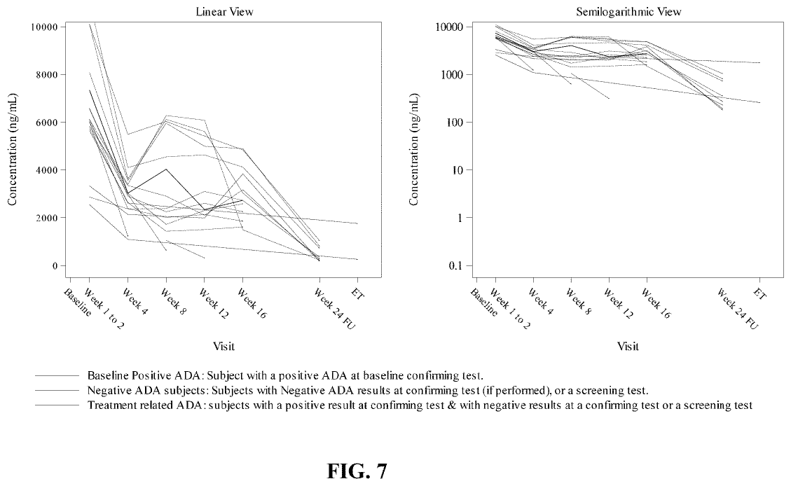

Overall incidence of ADA formation in the total ITT population was limited, as only one subject out of 20 presented treatment related ADA at week 4. No ADA-positive sample had neutralizing antibodies. Importantly, serum nemolizumab concentrations in ADA-positive subjects were not different from those in ADA-negative subjects, as can be observed in FIG. 7.

Analysis of Efficacy

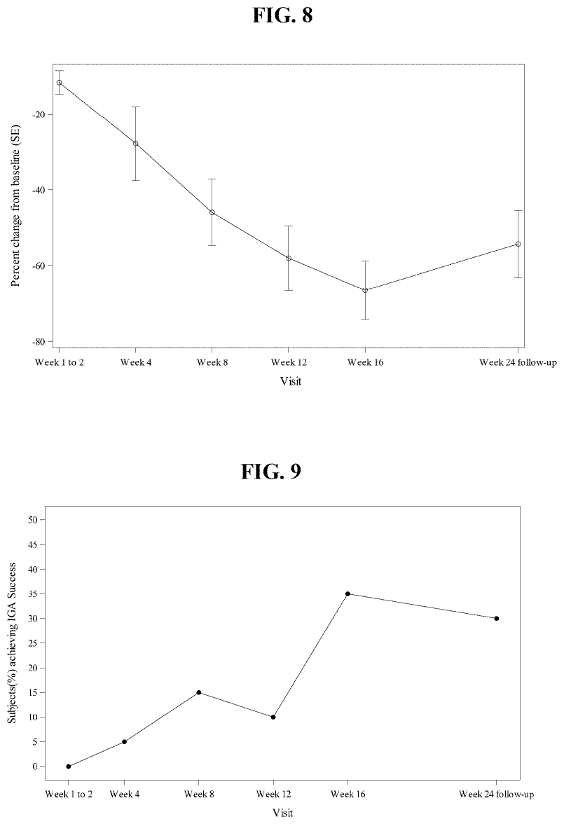

Eczema area and severity index (EASI). The mean (SD) change from baseline in EASI at Week 16 was −16.0 (9.9), with a percent change from baseline of −66.5% (32.5) using LOCF (Table 19). Over the course of the study, the EASI scores gradually improved up to Week 16. FIG. 8 displays the plot of percent change in EASI score over time.

TABLE 19
Summary of Eczema Area and Severity Index score
(last observation carried forward) Intent-to-treat population)

| | Nemolizumab 30 mg (N = 20) | | | |
|---|---|---|---|---|
| | | | Change from | Percent change |
| Analysis visit | Value | | baseline | from baseline |
| Baseline | | | | |
| n | 20 | | | |
| Mean (SD) | 25.2 | (7.2) | | |
| Median | 23.6 | | | |
| Min-Max | 16.3-37.4 | | | |
| Week 16 | | | | |
| n | 18 | | 18 | 18 |
| Mean (SD) | 8.0 | (9.0) | −16.0 (9.9) | −66.5 (32.5) |

| | | | |
|---|---|---|---|
| Median | 4.40 | −16.08 | −77.35 |
| Min-Max | 0-36.0 | −32.1-2.4 | −100.0-7.1 |

Note:
N = number of subjects in the population;
n = number of subjects with available data
Max = maximum;
Min = minimum;
SD = standard deviation.

Investigator's global assessment. Improvements were observed beginning on Week 4 through Week 16 (Table 20). At Week 16, 7 (35.0%) subjects achieved IGA success (defined as subjects with 0 [clear] or 1 [almost clear] and at least 2-grade improvement from baseline data) using Non-responder imputation. Over the course of the study, number of subjects who achieved IGA success improved up to Week 16. FIG. 9 displays a plot of the proportion of subjects achieving investigator global assessment success over time for the ITT population.

TABLE 20
Summary of subjects achieving Investigator’s
Global Assessment success (non responder)
(Intent to treat population)

| | Nemolizumab 30 mg |
|---|---|
| Analysis visit | (N = 20) n (%) |
| Week 1 to 2 | 0 |
| Week 4 | 1 (5.0) |
| Week 8 | 3 (15.0) |
| Week 12 | 2 (10.0) |
| Week 16 | 7 (35.0) |
| Week 24 follow-up | 6 (30.0) |

N = number of subjects in the population;
n = number of subjects with available data;
IGA = investigator global assessment
Notes:
Subjects with missing data or data collected after rescue medication intake were considered as subjects who did not reach IGA success (Non-Responder). Percentage (%) was calculated using the number of subjects in population as the denominator. Baseline was the last available measurement prior to or on the first treatment. If date of treatment is missing, the date of enrollment was be used instead. IGA success was defined as subjects with 0 (clear) or 1 (almost clear) and at least 2-grade improvement from baseline data.

Body surface area (BSA). The mean (SD) change from baseline in BSA of AD involvement at Week 16 was −19.9 (17.0) with a percent change from baseline of −53.3 (39.8) using LOCF (Table 21). Over the course of the study, the BSA of AD involvement improved up to Week 16.

TABLE 21
Summary of body surface area of atopic dermatitis involvement (last
observation carried forward) (Intent to treat population)

| | Nemolizumab 30 mg (N = 20) | | | | | |
|---|---|---|---|---|---|---|
| | | | Change from | | Percent change | |
| Analysis visit | Value | | baseline | | from baseline | |
| Baseline | | | | | | |
| n | 20 | | | | | |
| Mean (SD) | 41.3 | (12.6) | | | | |
| Median | 39.5 | | | | | |
| Min-Max | 23-60 | | | | | |
| Week 16 | | | | | | |
| n | 18 | | 18 | | 18 | |
| Mean (SD) | 19.4 | (19.0) | −19.9 | (17.0) | −53.3 | (39.8) |

| | | | |
|---|---|---|---|
| Median | 13.0 | −21.5 | −57.73 |
| Min-Max | 0 60 | −53-10 | −100-25.0 |

Note:
N = number of subjects in the population;
n = number of subjects with available data
Max = maximum;
Min = minimum;
SD = standard deviation.
AD = Atopic Dermatitis;
LOCF = Last Observation Carried Forward.
Baseline was the last available measurement prior to or on the first treatment. If date of treatment was missing, the date of enrollment was used instead. Only the missing post-baseline values or values after use of rescue medication are imputed using LOCF, but baseline values was not used for imputation.

Summary of scoring atopic dermatitis (SCORAD). The mean (SD) change from baseline in SCORAD scores at Week 16 was −31.2 (19.8) with a percent change from baseline of −50.1 (27.0) using LOCF (Table 22). Over the course of the study, the SCORAD scores improved up to Week 16.

TABLE 22
Summary of scoring atopic dermatitis scores (last
observation carried forward) (intent to treat population)

| | Nemolizumab 30 mg (N = 20) | | | | | |
|---|---|---|---|---|---|---|
| | | | Change from | | Percent change | |
| Analysis visit | Value | | baseline | | from baseline | |
| Baseline | | | | | | |
| n | 20 | | | | | |
| Mean (SD) | 63.0 | (11.3) | | | | |
| Median | 60.8 | | | | | |
| Min-Max | 45.2-82.3 | | | | | |
| Week 16 | | | | | | |
| n | 18 | | 18 | | 18 | |
| Mean (SD) | 30.0 | (16.9) | −31.2 | (19.8) | −50.1 | (27.0) |

| | | | |
|---|---|---|---|
| Median | 30.1 | −27.9 | −47.9 |
| Min-Max | 0.0-70.9 | −81.2-0.3 | −100.0-0.4 |

Note:
N = number of subjects in the population;
n = number of subjects with available data
Max = maximum;
Min = minimum;
SD = standard deviation;
LOCF = Last Observation Carried Forward.
Baseline was the last available measurement prior to or on the first treatment. If date of treatment was missing, the date of enrollment was used instead. Only the missing post-baseline values or values after use of rescue medication are imputed using LOCF, but baseline values were not used for imputation.

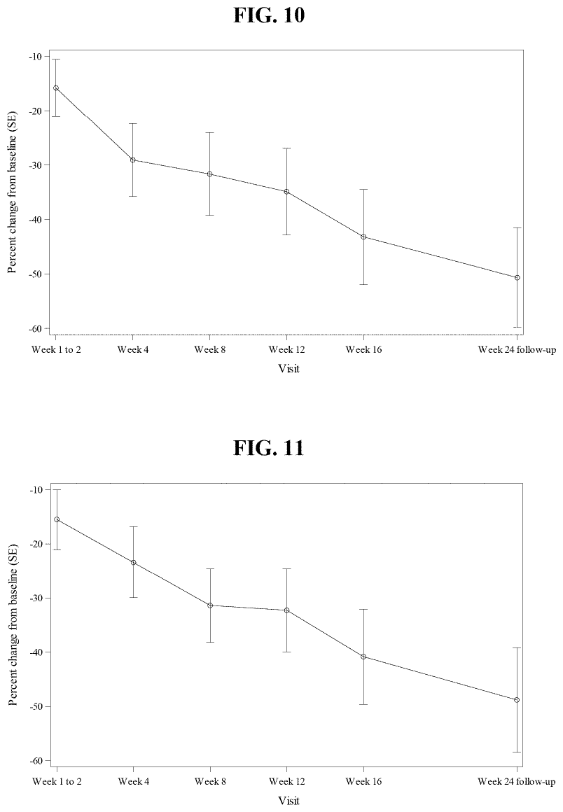

Weekly peak pruritus numeric rating scale score. The mean (SD) change from baseline in the weekly average of PP NRS at Week 16 was −3.1 (2.8) with a percent change from baseline of −43.2 (37.0) using LOCF (Table 23). Over the course of the study, the weekly average of PP NRS improved up to Week 16. FIG. 10 displays a plot of percent change from baseline in weekly average of PP NRS score over time for the ITT population.

TABLE 23
Summary of weekly average of peak pruritus numeric rating scale
score (last observation carried forward) (Intent to treat population)

| | Nemolizumab 30 mg (N = 20) | | | | | |
|---|---|---|---|---|---|---|
| | | | Change from | | Percent change | |
| Analysis visit | Value | | baseline | | from baseline | |
| Baseline | | | | | | |
| n | 18 | | | | | |
| Mean (SD) | 6.9 | (1.7) | | | | |
| Median | 7.5 | | | | | |
| Min-Max | 4.0-9.4 | | | | | |
| Week 16 | | | | | | |
| n | 19 | | 18 | | 18 | |
| Mean (SD) | 3.8 | (2.6) | −3.1 | (2.8) | −43.2 | (37.0) |

| | | | |
|---|---|---|---|
| Median | 3.7 | −3.1 | -47.2 |
| Min-Max | 0.0-9.5 | −8.3-1.0 | -100.0-12.1 |

Note:
N = number of subjects in the population; n=number of subjects with available data
Max = maximum;
Min = minimum;
SD = standard deviation;
LOCF = Last Observation Carried Forward.
Baseline was the last available measurement prior to or on the first treatment. If date of treatment was missing, the date of enrollment was used instead. Scale of itch intensity is in a range of 0 (no itch) to 10 (worst itch imaginable). Only the missing post-baseline values or values collected after use of rescue medication are imputed using LOCF, but baseline values are not used for imputation.

Weekly average of average pruritus numeric rating scale score. The mean (SD) change from baseline in the weekly average of AP NRS at Week 16 was −2.6 (2.6) with a percent change from baseline of −40.9 (37.3) using LOCF (Table 24). Over the course of the study, the weekly average of AP NRS improved up to Week 16. FIG. 11 displays a plot of percent change from baseline in weekly average of AP NRS score over time for the ITT population.

TABLE 24
Summary of weekly average of average pruritus numeric rating scale
score (last observation carried forward) (Intent-to-treat population)

| | Nemolizumab 30 mg (N = 20) | | | | | |
|---|---|---|---|---|---|---|
| | | | Change from | | Percent change | |
| Analysis visit | Value | | baseline | | from baseline | |
| Baseline | | | | | | |
| n | 18 | | | | | |
| Mean (SD) | 6.2 | (1.7) | | | | |
| Median | 6.2 | | | | | |
| Min-Max | 3.3-8.4 | | | | | |
| Week 16 | | | | | | |
| n | 19 | | 18 | | 18 | |
| Mean (SD) | 3.6 | (2.5) | −2.6 | (2.6) | −40.9 | (37.3) |

| | | | |
|---|---|---|---|
| Median | 3.8 | −2.3 | −41.4 |
| Min-Max | 0.0-9.2 | −8.0-0.92 | −100.0-15.2 |

Note:
N = number of subjects in the population;
n = number of subjects with available data
Max = maximum;
Min = minimum;
SD = standard deviation;
LOCF = Last Observation Carried Forward.
Baseline was the last available measurement prior to or on the first treatment. If date of treatment was missing, the date of enrollment was used instead. Scale of itch intensity is in a range of 0 (no itch) to 10 (worst itch imaginable). Only the missing post-baseline values or values collected after use of rescue medication are imputed using LOCF, but baseline values are not used for imputation.

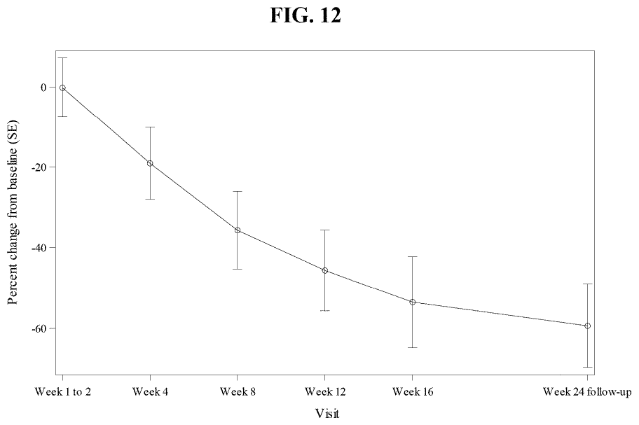

Weekly average of sleep disturbance numeric rating scale score. The weekly average of sleep disturbance improved from baseline to Week 16, with a mean (SD) change of 3.1 (3.2) and percent change from baseline of −53.5% (47.8) using LOCF (Table 25). FIG. 12 displays a plot of percent change from baseline in weekly average sleep disturbance NRS score over time for the ITT population.

TABLE 25
Summary of weekly average sleep disturbance numeric rating scale
score (last observation carried forward) (Intent to treat population)

| | Nemolizumab 30 mg (N = 20) | | | | | |
|---|---|---|---|---|---|---|
| | | | Change from | | Percent change | |
| Analysis visit | Value | | baseline | | from baseline | |
| Baseline | | | | | | |
| n | 18 | | | | | |
| Mean (SD) | 5.6 | (2.3) | | | | |
| Median | 5.9 | | | | | |
| Min-Max | 0.63-8.88 | | | | | |
| Week 16 | | | | | | |
| n | 19 | | 18 | | 18 | |
| Mean (SD) | 2.7 | (2.7) | −3.1 | (3.2) | -53.5 | (47.8) |

| | | | |
|---|---|---|---|
| Median | 1.8 | −2.5 | -57.8 |
| Min-Max | 0.0-9.2 | −8.3-2.0 | −100.0-48.8 |

Note:
N = number of subjects in the population;
n = number of subjects with available data
Max = maximum;
Min = minimum;
SD = standard deviation;
LOCF = Last Observation Carried Forward.
Baseline was the last available measurement prior to or on the first treatment. If date of treatment was missing, the date of enrollment was used instead. Only the missing post-baseline values or values collected after use of rescue medication are imputed using LOCF, but baseline values are not used for imputation.

Topical atopic dermatitis medication-free days. The mean (SD) number of medication-free days improved over the course of the study, from 3.5 (2.25) days at baseline to 10.7 (5.82) days from Week 12 to Week 16, and increased again to 23.4 (11.88) days between Week 16 and Week 24.

Dermatology life quality index. Both DLQI and cDLQI mean (SD) scores showed improvement over the course of the study. DLQI improved from 11.8 (3.11) to 3.0 (2.58) at Baseline and Week 16, respectively. cDLQI improved from 110.6 (5.42) to 6.2 (5.29) at Baseline and Week 16, respectively.

Biomarker Analysis

Approach. Biomarker samples were available from 19 AD subjects, aged 13 to 17 years, who were treated with nemolizumab. For plasma analyses, blood samples were collected at Baseline, Week 8, and Week 16 post-treatment. For SC analyses, at Baseline, lesional and non-lesional samples were collected, while at Week 16 post-treatment only lesional samples were collected. For biomarker evaluation, 30 proteins were measured in plasma and SC samples. These included CCL13, CCL2, CCL20, CCL22, CCL26, CCL27, CX3CL1, CXCL6, EGF, FAS L, Galectin-1, IL-1RA, CXCL8/IL-8, IL-11, IL-21, PLA2G7, CCL17/TARC, CCL21, IL-1α, IL-2, Oncostatin M, SP-D, VEGF-A, CCL5, Cystatin C, CCL18, Adiponectin, TGFb1, IL-31, and IgE (plasma only).

IL-6 and IL-18 analyses were not performed on plasma or SC samples as the above mentioned 30 biomarkers were considered more relevant to AD, while the 2 cytokines are considered more as markers of general inflammation. Further, preliminary analyses from a previous study in atopic dermatitis (SPR114322, Phase 2b), revealed that these cytokines were not predictive of responsiveness. Finally, due to the limited quantity of the SC samples, only protein biomarker analyses could be performed and no EOS ceramide, or mRNA and miRNA analyses could be performed.

Clinical parameters—EASI-75, IGA, and PP-NRS at Week 16 were considered the main outcomes of the biomarker substudy. Clinical responders using these parameters were defined as:
EASI-75 responders: at least 75% decrease in EASI score from baseline.
IGA-01 responders: IGA 1.
PP-NRS responder: At least 4-point decrease in Peak Pruritus Maximum Itch NRS from baseline.

Stratum Corneum:

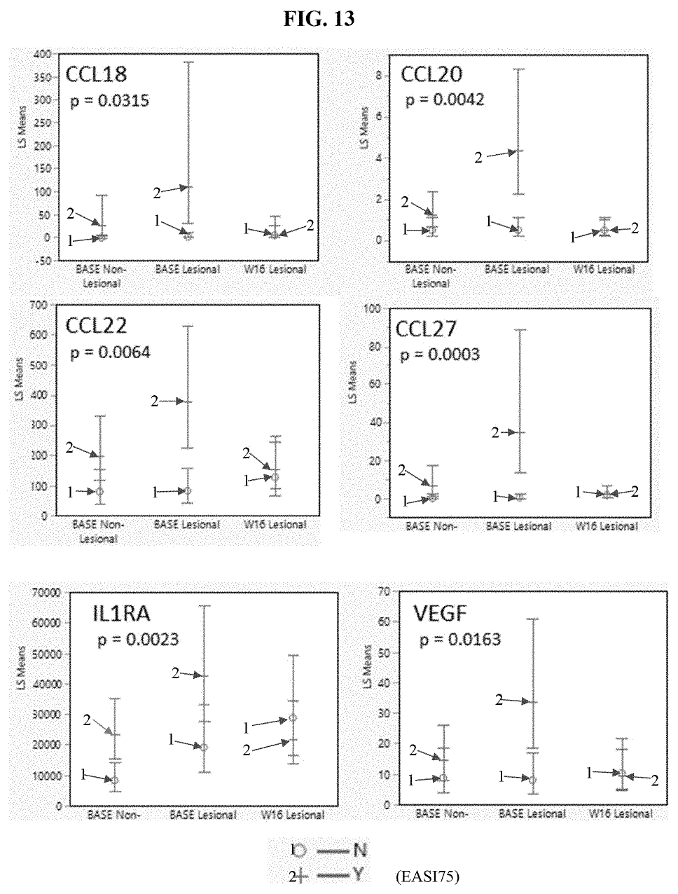

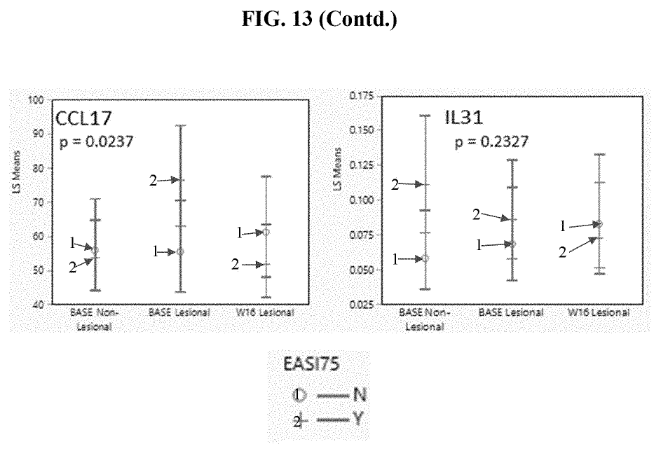

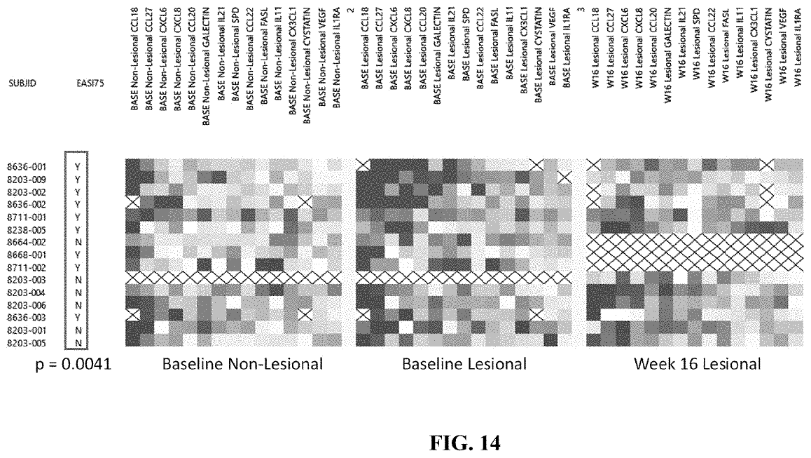

Supervised analysis—mixed model repeated measures (MMRM). Six proteins significantly associated with the EASI-75 outcome: CCL18, CCL20, CCL22, CCL27, IL1RA with false discovery rate (FDR)<0.05 and VEGF with FDR<0.10 (FIG. 13), where the FDR considered is the minimum adjusted p value (interaction, main effect). Although insignificant with respect to the FDR criterion, CCL17 and IL31 are also presented due to the particular interest in these proteins (FIG. 13). Nevertheless, CCL17 expression was also significantly different between lesional and non lesional skin in EASI75 responders and was concomitantly reduced post treatment in week 16 lesional skin. The fold change between lesional and non-lesional samples at baseline was 1.9 to 3.5 higher in responders than in nonresponders for 4 out of the 6 proteins, namely CCL20, CCL22, CCL27, and VEGF. This difference is statistically significant (as shown in FIG. 13). These data suggest that these 4 proteins could be used as potential biomarkers for clinical responsiveness (FIG. 14). No changes in the measured protein biomarkers were found to be associated with IGA and PP-NRS at Week 16. Because only 10 subjects were documented at Week 16, PP-NRS was also considered at Week 12 with 14 documented subjects. A significant association with CCL17 and CCL20 was observed.

Unsupervised analysis, non-negative tensor factorization. Unsupervised analysis was performed on 15 subjects with at least 1 documented outcome (EASI-75, IGA, PP-NRS) and proteomic profiles in at least 1 condition (baseline non-lesional, baseline lesional, or Week 16 lesional). The association between the non-negative tensor factorization (NTF)-provided subject ranking and EASI-75 was significant (p=0.0224). Fifteen proteins (including the 6 proteins found by the supervised analyses) contributed the most to the achieved ranking: CCL18, CCL20, CCL22, CCL27, CX3CL1, CXCL6, CXCL8, CYSTATIN, FASL, GALECTIN, IL11, IL21, IL1RA, SPD, VEGF (Error! Reference source not found.). When restricting the analyses to these 15 proteins, the association between NTF-provided subject ranking and EASI-75 was even more significant (p=0.0041). Like the supervised analyses, IGA-01 scores were not associated with subject ranking (p=0.5493). The association p-value with PP-NRS could not be calculated due to unstable estimates caused by the low sample size (only 10 subjects documented). When using PP-NRS at week 12, the association was still insignificant. The results of the analysis are shown in the table below:

| | Fraction Base | Fraction Base Non- | Fraction W16 |
|---|---|---|---|
| | Lesional (ppm) | Lesional (ppm) | Lesional (ppm) |
| CCL18 | 319.85 | 130.43 | 39.43 |
| CCL20 | 16.52 | 10.53 | 5.48 |
| CCL22 | 1663.32 | 1629.96 | 1495.42 |
| CCL27 | 69.91 | 30.94 | 22.96 |
| CX3CL1 | 3181.01 | 4458.51 | 3727.43 |
| CXCL6 | 43.68 | 27.85 | 25.55 |
| CXCL8 | 661.22 | 306.61 | 248.58 |
| CYSTATIN | 84351.26 | 68828.98 | 39914.58 |
| FASL | 4.48 | 3.38 | 2.91 |
| GALECTIN | 2333.92 | 1731.12 | 962.21 |
| IL1RA | 229241.75 | 206501.19 | 255810.65 |
| IL11 | 15183.41 | 15053.38 | 18778.70 |
| IL21 | 6.76 | 5.00 | 3.43 |
| SPD | 472.86 | 369.82 | 764.48 |
| VEGF | 162.77 | 154.75 | 100.95 |

Plasma: neither the supervise nor the unsupervised analysis showed significant correlation between the measured proteins and clinical scores.

Safety Evaluation Results.

Extent of exposure. The mean duration of study treatment was 68.7 days with a median of 85.0 days. The mean total dose planned and administered during the study was 131.7 mg (10000 compliance). A summary of exposure to study drug can be found in Table 26.

TABLE 26
Summary of extent of exposure (Safety population)

| | Nemolizumab 30 mg (N = 18) | |
|---|---|---|
| Total dose administered | | |
| during the study (mg) | | |
| | n | 18 |
| | Mean | 131.7 |
| | SD | 32.76 |
| | Median | 150.0 |
| | Min | 60 |
| | Max | 150 |
| Total dose planned | | |
| during the study (mg) | | |
| | n | 18 |
| | Mean | 131.7 |
| | SD | 32.76 |
| | Median | 150.0 |
| | Min | 60 |
| | Max | 150 |
| Treatment duration (days) | | |
| | n | 18 |
| | Mean | 68.7 |
| | SD | 31.5 |
| | Median | 85.0 |
| | Min | 1 |
| | Max | 91 |
| Treatment compliance (%) | | |
| | n | 18 |
| | Mean | 100.0 |
| | SD | 0.0 |
| | Median | 100.0 |
| | Min | 100 |
| | Max | 100 |

N = Number of subjects in population;
n = Number of subjects with data available;
SD = Standard Deviation.
Study duration is calculated as the date of last visit minus the date of first visit plus one.

Adverse events. A total of 6 (33.3%) subjects experienced 14 TEAEs. Of these subjects, 3 (16.7%) were reported to have 10 TEAEs related to the study drug. Most of the reported TEAEs by maximum severity were either mild (6 events in3 [16.7%] subjects) or moderate (4 events in 1 [5.6%] subject). Two (11.1%) subjects had 4 TEAEs that were considered severe. Two of these TEAEs were serious (i.e., infectious eczematoid dermatitis and right leg edema with staphylococcal skin infection). Four treatment-emergent AESIs reported in this study were experienced by the same 2 subjects. Each of these 2 subjects (8667-001 and 8668-001) had 1 serious AESI (infectious eczematoid dermatitis and right leg edema with staphylococcal skin infection, respectively) and 1 nonserious AESI (bilateral leg edema and bilateral lower extremity distal edema, respectively). All AESIs were considered to be study drug related. These same 2 subjects also had 7 related AEs that led to permanent discontinuation of the study drug (Subject 8667-001 [atopic dermatitis, bilateral leg oedema, bilateral leg pain, and rash pustula] and Subject 8668-001 [bilateral leg oedema, right leg oedema, and staphylococcal skin infection]). No TEAES led to discontinuation from the study.

As presented in Table 27, six of 18 subjects (33.3%) experienced at least 1 TEAE. One subject had two SAEs and one subject had one SAE for a total of 3 SAEs. The same 2 subjects had 3 TEAEs that were assessed as severe. Seven TEAEs reported for these 2 subjects led to permanent discontinuation of the study drug. Four AESIs were reported for 2 subjects. No deaths were reported during the study.

TABLE 27
Overall summary of treatment-emergent adverse events,
all causalities (Safety population)

| | Nemolizumab 30 mg | | |
|---|---|---|---|
| | N = 18 | | |
| | n (%) | | Number of events |
| Any TEAE | 6 | (33.3) | 14 |
| Any TEAE related to study drug | 3 | (16.7) | 10 |
| Any serious TEAE | 2 | (11.1) | 3 |
| Any serious TEAE related to study | 2 | (11.1) | 3 |
| drug | | | |
| Any severe TEAE | 2 | (11.l) | 4 |
| Any TEAE leading to permanent | 2 | (11.l) | 7 |
| discontinuation from study drug | | | |
| Any treatment-emergent AESI | 2 | (11.l) | 4 |

AESI = adverse event of special interest;
TEAE = treatment-related adverse event

As presented in Table 28, the system organ class (SOC) with the most TEAES reported was Infections and infestations (5 events in 4 subjects). With the exception of peripheral edema, which was experienced by 2 subjects, all other AEs were experienced by 1 subject each.

TABLE 28
Treatment-emergent adverse events by system organ class
and preferred term, all causalities (Safety population)

| | Nemolizumab 30 mg | |
|---|---|---|
| System organ class | N = 18 | |
| Preferred term | n (%) | Number of events |
| Any TEAE | 6 (33.3) | 14 |
| General disorders and | 3 (16.7) | 4 |
| administration site conditions | | |
| Oedema peripheral | 2 (11.1) | 3 |
| Fatigue | 1 (5.6) | 1 |
| Infections and infestations | 4 (22.2) | 5 |
| Bronchitis | 1 (5.6) | 1 |
| Eczema infected | 1 (5.6) | 1 |
| Rash pustular | 1 (5.6) | 1 |
| Staphylococcus skin infection | 1 (5.6) | 1 |
| Upper respiratory tract infection | 1 (5.6) | 1 |
| Injury, poisoning and procedural | 1 (5.6) | 1 |
| complications | | |
| Arthropod bite | 1 (5.6) | 1 |
| Investigations | 1 (5.6) | 1 |
| Blood creatine phosphokinase increased | 1 (5.6) | 1 |
| Musculoskeletal and connective tissue | 1 (5.6) | 1 |
| disorders | | |
| Pain in extremity | 1 (5.6) | 1 |
| Psychiatric disorders | 1 (5.6) | 1 |
| Depression | 1 (5.6) | 1 |
| Skin and subcutaneous tissue disorders | 1 (5.6) | 1 |
| Dermatitis atopic | 1 (5.6) | 1 |

Adverse events by relationship to study drug. Three subjects experienced 10 TEAEs that were considered to be related to study drug by the Investigator. Serious TEAEs, severe TEAEs, TEAEs of special interest, and TEAEs that led to permanent discontinuation from study drug that were considered to be related to study drug were experienced by 2 (11.10%) subjects each. No subject experienced a TEAL that was considered to be related to study procedure.

Adverse events by severity. The majority of reported TEAEs by maximum severity were mild (6 events in 3 [16.70%] subjects), severe (4 events in 2 [11.10%] subjects), or moderate (4 events in 1 [5.60%] subject). Among the 4 moderate events, the most often reported TEAL was oedema peripheral (2 events in 1 [5.60%] subject) in the general disorders and administration site conditions SOC. Four severe events (oedema peripheral, eczema infected, staphylococcal skin infection, and dermatitis atopic) were reported in 2 subjects. One subject (8667-001) experienced eczema infected and dermatitis atopic in the infections and infestations and skin and subcutaneous skin disorders SOCs, respectively, while the other subject (8668-001) experienced staphylococcal skin infection and oedema peripheral in the infections and infestations and general disorders and administration site conditions SOCs, respectively.

Deaths, other serious AEs, and other significant AEs. No deaths occurred during the study. Two subjects experienced 3 SAEs (Eczema infected, oedema peripheral, and staphylococcus skin infection), all of which were considered drug related. A summary of serious TEAEs is presented in Table 29.

TABLE 29
Serious treatment-emergent adverse events of special interest by system
organ class and preferred term (Safety population)

| | Nemolizumab 30 mg | |
|---|---|---|
| System organ class | N = 18 | |
| Preferred term | n (%) | Number of events |
| Any Serious TEAE | 2 (11.1) | 3 |
| General disorders and administration site | 1 (5.6) | 1 |
| conditions | | |
| Oedema peripheral | 1 (5.6) | 1 |
| Infections and infestations | 2 (11.1) | 2 |
| Eczema infected | 1 (5.6) | 1 |
| Staphylococcus skin infection | 1 (5.6) | 1 |

N = Number of subjects in population;
n = Number of subjects with data available.
Notes:
Percentage (%) is calculated using the number of subjects in population as the denominator. Adverse events are coded using MedDRA version 21.1. Treatment-emergent adverse events (TEAEs) are defined as events with an onset date on or after date of study treatment administration.

Adverse events of special interest. Four treatment-emergent AESIs reported in this study were experienced by the same 2 subjects. Each of these 2 subjects (8667-001 and 8668-001) had 1 serious AESI (infectious eczematoid dermatitis and right leg edema with staphylococcal skin infection, respectively) and 1 nonserious AESI (bilateral leg edema and bilateral lower extremity distal edema, respectively). All AESIs were considered to be study drug related. A summary of TEAEs of special interest is presented in Table 30.

TABLE 30
Treatment-emergent adverse events of special interest by system organ
class and preferred term, all causalities (Safety population)

| | Nemolizumab 30 mg | |
|---|---|---|
| System organ class | N = 18 | |
| Preferred term | n (%) | Number of events |
| Any TEAE of special interest | 2 (11.1) | 4 |
| General disorders and administration site | 2 (11.1) | 2 |
| conditions | | |
| Oedema peripheral | 2 (11.1) | 2 |
| Infections and infestations | 2 (11.1) | 2 |
| Eczema infected | 1 (5.6) | 1 |
| Staphylococcus skin infection | 1 (5.6) | 1 |

N = Number of subjects in population;
n = Number of subjects with data available.
Notes:
Percentage (%) is calculated using the number of subjects in population as the denominator. Adverse events are coded using MedDRA version 21.1. Treatment-emergent adverse events (TEAEs) are defined as events with an onset date on or after date of study treatment administration.

Treatment-emergent adverse events leading to discontinuation. Seven TEAEs leading to permanent study drug discontinuation were experienced by the same 2 subjects as the SAEs and AESIs (Subject 8667 001 [atopic dermatitis, bilateral leg oedema, bilateral leg pain, and rash pustula] and Subject 8668 001 [bilateral leg oedema, right leg oedema, and staphylococcal skin infection]). No TEAEs led to subject discontinuation from the study.

Clinical Laboratory Evaluation Results:

Hematology. Small changes from baseline in hematology parameters were observed during the study. No notable trends were observed in shifts from baseline among hematology laboratory values. For those hematology values that were recorded post-baseline, almost all that were normal at baseline remained normal at post-baseline timepoints.

Chemistry. Small changes from baseline in chemistry parameters were observed during the study. No notable trends were observed in shifts from baseline among chemistry laboratory values. For those chemistry values that were recorded post-baseline, almost all that were normal at baseline remained normal at post-baseline timepoints.

Urinalysis. No PCS urinalysis laboratory results were observed.

Vital signs, physical findings, and other observations related to safety:

Vital signs. Potentially clinically significant vital signs occurred in 5 subjects during the study. One subject had a PCS diastolic blood pressure value (>90 mmHg and an increase of >10 mmHg from baseline) at Week 12, temperature (<35° C.) at Week 1 to 2, and changes in weight (<95% change from baseline) at Weeks 16 and 24. Two other subjects had a PCS change in weight (>95% of change from baseline) beginning at Week 8 through Week 24, and at Week 12, respectively. One subject had PCS changes in weight (>105% of change from baseline) at Week 8 and 4 subjects had PCS changes in weight (>105% of change from baseline) at Weeks 12, 16, and 24, respectively. No notable trends were observed among vital sign values.

Electrocardiogram data. No notable trends were observed among ECG parameters and no clinically significant abnormalities were reported at screening or Week 16.

Physical examination findings. A clinically significant skin abnormality (atopic dermatitis) was reported for 1 subject at Week 4, 7 subjects at Week 8, and 1 subject at Week 16. No notable trends were observed among physical examination values.

Respiratory assessment results. One subject (8614-001) with a history of asthma had an abnormal PEF (77.6% of predicted value) at Screening, which was later identified as a major protocol deviation, and the site was instructed to discontinue dispensing any study drug immediately. At Screening, the subject's ACT score was normal (25). The subject returned for all subsequent safety procedures, as was requested by the Sponsor. The PEF values remained abnormal until the early termination visit, at which time PEF was 104.8% of the predicted value. A summary of abnormal PEF is presented in Table 31.

TABLE 31
Summary of abnormal peak expiratory flow (Safety population)

| | | Nemolizumab 30 mg | |
|---|---|---|---|
| Visit | PEF Result | N = 18 | |
| Screening | n | 17 | |
| | PEF <80% of predictive value | 2 | (11.8%) |
| Baseline | n | 17 | |
| | PEF <80% of predictive value | 2 | (11.8%) |
| Week 1 to 2 | n | 10 | |
| | PEF <80% of predictive value | 1 | (10.0%) |
| Week 4 | n | 8 | |
| | PEF <80% of predictive value | 1 | (12.5) |

PEF = peak expiratory flow

One subject (8668-001) had an abnormal asthma control test (ACT) score of 13 at Screening, which was later identified as a major protocol deviation. An ACT score≤19 suggests asthma which may not be adequately controlled. However, a baseline loading dose was administered in error. The subject's PEF was normal at screening (87.7% of predicted value). The subject returned for all subsequent safety procedures, as was requested by the Sponsor. The ACT scores were normal at all visits except for Week 12, at which the ACT score was 15. Scores for ACT were normal at all timepoints for all other subjects. No notable trends were observed among respiratory values. A summary of ACT Score≤19 is presented in Table 32.

TABLE 32
Summary of asthma control test score ≤19 (safety population)

| | | Nemolizumab | |
|---|---|---|---|
| Visit | ACT Result | 30 mg N = 18 | |
| Screening | n | 9 | |
| | ACT Score £19 | 1 | (11.1) |
| Baseline | n | 9 | |
| | ACT Score £19 | 0 | |
| Week 1 to 2 | n | 10 | |
| | ACT Score £19 | 0 | |
| Week 4 | n | 8 | |
| | ACT Score £19 | 0 | |
| Week8 | n | 7 | |
| | ACT Score £19 | 0 | |
| Week 12 | n | 7 | |
| | ACT Score £19 | 1 | (14.3) |

ACT = asthma control test

Safety conclusions. Six (33.3%) subjects experienced 14 TEAEs, of which most were either mild or moderate by maximum severity. Two subjects experienced 3 SAEs (eczema infected, oedema peripheral, and staphylococcus skin infection), all of which were considered drug related. The same 2 subjects who experienced SAEs experienced 4 AESIs; each subject had 1 serious and 1 nonserious AESI. Seven TEAEs led to permanent study drug discontinuation and were experienced by the same 2 subjects as the SAEs and AESIs. No TEAEs led to discontinuation of the subject from the study. No deaths occurred during the study. No notable trends were observed among laboratory, vital signs, and respiratory assessments over time.

What is claimed:

1. A method of treating or preventing atopic dermatitis (AD) in a subject, comprising administering to a subject with AD an anti-IL-31RA antibody, wherein the subject has skin lesions in which expression of at least one of CCL20, CCL22, CCL27, VEGF, IL1RA, and CCL18 are up-regulated compared to non-lesional skin of an individual without AD or non-lesional skin of the subject.

2. The method of claim 1, wherein CCL20, CCL22, CCL27, and VEGF are up-regulated in the skin lesion of the subject compared to non-lesional skin of an individual without AD or non-lesional skin of the subject.

3. The method of claim 1, wherein expression of CCL20, CCL22, CCL27, VEGF, IL1RA, and/or CCL18 is determined by RT-qPCR, RT-PCR, RNA-seq, Northern blotting, Serial Analysis of Gene Expression (SAGE), DNA or RNA microarrays, Western blotting, ELISA, surface plasmon resonance, or mass spectrometry.

4. The method of claim 1, wherein one, two, three, four, five, or six of CCL20, CCL22, CCL27, VEGF, IL1RA, and CCL18 are up-regulated in the skin lesion of the subject compared to non-lesional skin of an individual without AD or non-lesional skin of the subject.

5. The method of claim 1, wherein the expression of at least one of CCL20, CCL22, CCL27, VEGF, IL1RA, and CCL18 is altered or reduced within 2 weeks, 4 week, 8 weeks, 10 weeks, 12 weeks, 14 weeks, 16 week, 18 weeks, or 20 weeks of the administration of the anti-IL-31RA antibody compared a baseline level of expression in the skin lesion of the subject prior to administration of the anti-TL-31RA antibody.

6. The method of claim 1, wherein expression of CCL20 is reduced by at least about 3-fold, expression of CCL22 is reduced by at least about 1.1-fold, expression of CCL27 is reduced by at least about 3-fold, expression of VEGF is reduced by at least about 1.6-fold, expression of CCL18 is reduced by at least about 8-fold, and/or expression of IL1RA is increased by at least about 1.1-fold.

7. The method of claim 1, wherein the subject achieves at least a 66.5% decrease in Eczema Area and Severity Index (EASI) scoring following administration of the anti-IL-31RA antibody.

8. The method of claim 1, wherein the subject achieves at least a 43.2% decrease in peak pruritis numeric rating scale (PP-NRS) scoring following administration of the anti-IL-31RA antibody.

9. The method of claim 1, wherein the subject experiences improved sleep following administration of the anti-IL-31RA antibody.

10. The method of claim 1, wherein the subject is an adult.

11. The method of claim 1, wherein the subject is an adolescent, optionally between the ages of 12 and 17 years old.

12. The method of claim 1, wherein the anti-IL-31RA antibody is administered subcutaneously.

13. The method of claim 1, wherein the anti-IL-31RA antibody is administered once every two to four weeks.

14. The method of claim 1, wherein the anti-TL-31RA antibody is administered at a dose of about 0.01 mg/kg to about 10 mg/kg.

15. The method of claim 1, wherein the anti-TL-31RA antibody is administered at a dose of about 10 mg to about 90 mg.

16. The method of any claim 1, wherein the anti-IL-31RA antibody comprises a heavy chain variable region comprising a HCDR1 comprising SEQ ID NO: 8, a HCDR2 comprising SEQ ID NO: 9, and a HCDR3 comprising SEQ ID NO: 10, and a light chain variable region comprising a LCDR1 comprising SEQ ID NO: 12, a LCDR2 comprising SEQ ID NO: 13, and a LCDR3 comprising SEQ ID NO: 14.

17. The method of claim 1, wherein the anti-IL-31RA antibody is nemolizumab or a fragment or variant thereof.

18. The method of claim 17, wherein the anti-IL-31RA antibody is nemolizumab.

18. The method of claim 1, wherein the anti-IL-31RA antibody is administered subcutaneously at a loading dose of 60 mg, followed by a dose of 30 mg every four weeks for at least 12, 14, 16, or 18 weeks.

20. The method of claim 1, wherein the anti-IL-31RA antibody is administered according to a flat dosing regimen.

21. The method of claim 1, wherein the anti-IL-31RA antibody is administered according to a loading dose regimen.

22. A method of altering or reducing expression of at least one of CCL20, CCL22, CCL27, VEGF, IL1RA, and CCL18 in a skin lesion of a subject with atopic dermatitis (AD) comprising administering to a subject with AD an anti-TL-31RA antibody, wherein at least one of CCL20, CCL22, CCL27, VEGF, IL1RA, and CCL18 are up-regulated in a skin lesion of the subject compared to non-lesional skin of an individual without AD or non-lesional skin of the subject, and wherein administration of the anti-TL-31RA antibody reduces the expression of at least one of CCL20, CCL22, CCL27, VEGF, IL1RA, and CCL18 in a skin lesion.

23. A method of reducing expression of an inflammatory biomarker in the skin of a subject with atopic dermatitis (AD), comprising administering to a subject with AD an anti-IL-31RA antibody, thereby decreasing an inflammatory responses in the skin.

24. A method of diagnosing atopic dermatitis (AD), comprising detecting in a sample obtained from a subject suspected of having AD the expression level of at least one, at least two, at least three, at least four, at least five, or all six biomarker(s) selected from CCL20, CCL22, CCL27, VEGF, IL1RA, and CCL18, and comparing the expression level of the biomarkers to a reference level, wherein the reference level is the corresponding level of expression for each biomarker in a skin sample from an individual that does not have AD or a sample of non-lasional skin from the subject.

25. A method of determining whether a subject with atopic dermatitis (AD) will respond to treatment with an anti-IL-31RA antibody, comprising detecting in a sample obtained from a subject suspected of having AD the expression level of at least one, at least two, at least three, at least four, at least five, or all six biomarker(s) selected from CCL20, CCL22, CCL27, VEGF, IL1RA, and CCL18, and comparing the expression level of the biomarkers to a reference level, wherein the subject will respond to treatment if the expression level of the biomarkers is higher than the reference level, and wherein the reference level is the corresponding level of expression for each biomarker in a skin sample from an individual that does not have AD or a sample of non-lasional skin from the subject.

26. A method of determining whether a subject with atopic dermatitis (AD) is responding to treatment with an anti-IL-31RA antibody, comprising detecting in a post-treatment sample obtained from a subject with AD that has been administered at least one dose of an anti-IL-31RA antibody the expression level of at least one, at least two, at least three, at least four, at least five, or all six biomarker(s) selected from CCL20, CCL22, CCL27, VEGF, IL1RA, and CCL18, and comparing the expression level of the biomarkers to a baseline level of expression from a sample obtained from the same subject before treatment was commenced, wherein a decrease in the expression level of CCL20, CCL22, CCL27, VEGF, and/or CCL18 and/or an increase in the expression level of IL1RA is indicative of the subject responding to treatment.
